(19) United States
(12) Patent Application Publication
(10) Pub. No.: US 2007/0032457 A1
(43) Pub. Date: Feb. 8, 2007

(54) Combination therapy for cancer treatment

(76) Inventor: Lawrence M. Blatt, San Francisco, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/556,474

(22) PCT Filed: May 13, 2004
(86) PCT No.: PCT/US04/15031

§ 371 Date: Oct. 20, 2006

Related U.S. Application Data
(60) Provisional application No. 60/471,129, filed on May 16, 2003.
(60) Provisional application No. 60/471,249, filed on May 16, 2003.
(60) Provisional application No. 60/471,043, filed on May 16, 2003.
(60) Provisional application No. 60/471,179, filed on May 16, 2003.

(51) Int. Cl. A61K 38/21; A61K 31/66; A61K 39/395; A61K 31/56; A61K 31/4745; A61K 31/337

(52) U.S. Cl. 514/102; 424/85.7; 424/155.1; 514/283; 514/449; 514/171

(57) ABSTRACT

The present invention provides methods of treating cancer, the methods generally involving combination therapy. The methods are useful as primary cancer therapy, or as adjuvant therapy. The present invention further provides diagnostic methods for determining the responsiveness of a given tumor to treatment with a combination therapy.

### CROSS-REFERENCE TO RELATED APPLICATIONS

This application claims the priority of U.S. provisional application Ser. Nos. 60/471,129, filed May 16, 2003; 60/471,249, filed May 16, 2003; 60/471,043, filed May 16, 2003; and 60/471,179, filed May 16, 2003, which applications are incorporated herein by reference in their entirety.

### FIELD OF THE INVENTION

The present invention is in the field of cancer treatment, and in particular the use of IP-10 and pirfenidone or a pirfenidone analog in combination therapy for cancer treatment.

### BACKGROUND OF THE INVENTION

The leading therapies for cancer are currently surgery, radiation and chemotherapy. Chemotherapeutic approaches such as antitumor antibiotics, alkylating agents, nitrosourea compounds, vinca alkaloids, steroid hormones, and anti-metabolites form the bulk of therapies available to oncologists. Despite advances in the field of cancer treatment, cancer remains a major health problem.

As one example, ovarian cancer presents a major health problem for women worldwide, and the statistics on the 5-year survival rate are grim. About 79% of ovarian cancer patients survive one year after diagnosis, and about 53% survive longer than five years after diagnosis. If diagnosed and treated while the cancer has not spread outside the ovary, the five-year survival rate is 95%. However, only about 25% of all ovarian cancers are detected at this early stage. When ovarian cancer is found outside of the ovary, but is still contained within the pelvis, the five-year survival rate is 60% to 80%. Where the cancer has spread beyond the pelvis to the omentum and other areas within the abdomen and/or the cancer has spread to the lymph nodes, the average five-year survival rate is 25%. Finally, where the cancer has spread to the inside of the liver or spleen, and possibly other locations distant from the ovaries, the average five-year survival rate is less than 10%.

There is a need in the art for improved methods for treating cancer. The present invention addresses this need and provides related advantages.

Literature

U.S. Pat. No. 5,474,981; U.S. Pat. No. 5,871,723; U.S. Pat. No. 5,728,377; U.S. Pat. No. 5,935,567; U.S. Pat. No. 5,994,292; U.S. Pat. No. 6,153,600; U.S. Pat. No. 6,491,906; WO 01/62274; WO 99/46392; Luster and Leder (1996) J. Exp. Med 178:1057-1065; Tannenbaum et al. (1998) J. Immunol. 161:927-932; Narvaiza et al. (2000) J. Immunol. 164:3112-3122.

### SUMMARY OF THE INVENTION

The present invention provides methods of treating cancer, the methods generally involving combination therapy. The methods are useful as primary cancer therapy, or as adjuvant therapy. The present invention further provides diagnostic methods for determining the responsiveness of a given tumor to treatment with a combination therapy.

### FEATURES OF THE INVENTION

IP-10 and Pirfenidone Combination Therapy

The present invention features a method of treating cancer, generally involving combination therapy with effective amounts of IP-10 and pirfenidone or a pirfenidone analog. The methods are useful as primary cancer therapy, or as adjuvant therapy.

In one aspect, the invention features a method of treating cancer by administering to a cancer patient effective amounts of IP-10 and pirfenidone or pirfenidone analog and co-administering to the cancer patient an effective amount of at least one additional drug that is a biological response modifier.

In another aspect, the invention features a method of treating cancer by administering to a cancer patient effective amounts of IP-10 and pirfenidone or pirfenidone analog and co-administering to the cancer patient an effective amount of at least one additional antineoplastic drug.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is a chemotherapeutic agent.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is an anti-angiogenic agent.

In another aspect, the invention features a method of treating cancer by administering to a cancer patient effective amounts of IP-10 and pirfenidone or pirfenidone analog and co-administering to the cancer patient an effective amount of a Type I interferon receptor agonist. In some embodiments, the Type I interferon receptor agonist is an interferon-α (IFN-α). In other embodiments, the IFN-α is a PEGylated IFN-α.

In another aspect, the invention features a method of treating cancer by administering to a cancer patient effective amounts of IP-10 and pirfenidone or pirfenidone analog and co-administering to the cancer patient an effective amount of a Type II interferon receptor agonist. In some embodiments, the Type II interferon receptor agonist is an interferon-γ (IFN-γ).

In another aspect, the invention features a method of treating cancer by administering to a cancer patient effective amounts of IP-10 and pirfenidone or pirfenidone analog and co-administering to the patient effective amounts of a Type I interferon receptor agonist and a Type II interferon receptor agonist. In some embodiments, the Type I interferon receptor agonist is IFN-α and the Type II interferon receptor agonist is IFN-γ.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is an alkylating agent. In some embodiments, the alkylating agent is a nitrogen mustard. In other embodiments, the alkylating agent is an ethylenimine. In still other embodiments, the alkylating agent is an alkylsulfonate. In additional embodiments, the alkylating agent is a triazene. In further embodiments, the alkylating agent is a nitrosourea.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is an antimetabolite. In some embodiments, the antimetabolite is a folic acid analog, such as methotrexate. In other embodiments, the antimetabolite is a purine analog, such as mercaptopurine, thioguanine and axathioprine. In still other embodiments, the antimetabolite is a pyrimidine analog, such as 5FU, UFT, capecitabine, gemcitabine and cytarabine.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is a vinca alkyloid. In some embodiments, the vinca alkaloid is a taxane, such as paclitaxel. In other embodiments, the vinca alkaloid is a podophyllotoxin, such as etoposide, teniposide, ironotecan, and topotecan.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is an antineoplastic antibiotic. In some embodiments, the antineoplastic antibiotic is doxorubicin.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is a platinum complex. In some embodiments, the platinum complex is cisplatin. In other embodiments, the platinum complex is carboplatin.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is a tyrosine kinase inhibitor. In some embodiments, the tyrosine kinase inhibitor is a receptor tyrosine kinase (RTK) inhibitor, such as type I receptor tyrosine kinase inhibitors (e.g., inhibitors of epidermal growth factor receptors), type II receptor tyrosine kinase inhibitors (e.g., inhibitors of insulin receptor), type III receptor tyrosine kinase inhibitors (e.g., inhibitors of platelet-derived growth factor receptor), and type IV receptor tyrosine kinase inhibitors (e.g., fibroblast growth factor receptor). In other embodiments, the tyrosine kinase inhibitor is a non-receptor tyrosine kinase inhibitor, such as inhibitors of src kinases or janus kinases.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is an inhibitor of a receptor tyrosine kinase involved in growth factor signaling pathway(s). In some embodiments, the inhibitor is genistein. In other embodiments, the inhibitor is an epidermal growth factor receptor (EGFR) tyrosine kinase-specific antagonist, such as IRESSA™ gefitinib, TARCEVA™ erolotinib, or tyrphostin AG1478 (4-(3-chloroanilino)-6,7-dimethoxyquinazoline. In still other embodiments, the inhibitor is any indolinone antagonist of Flk-1/KDR (VEGF-R2) tyrosine kinase activity. In further embodiments, the inhibitor is any of the substituted 3-[(4,5,6,7-tetrahydro-1H-indol-2-yl)methylene]-1,3-dihydroindol-2-one antagonist of Flk-1/KDR (VEGF-R2), FGF-R1 or PDGF-R tyrosine kinase activity. In additional embodiments, the inhibitor is any substituted 3-[(3- or 4-carboxyethylpyrrol-2-yl)methylidenyl]indolin-2-one antagonist of Flt-1 (VEGF-R1), Flk-1/KDR (VEGF-R2), FGF-R1 or PDGF-R tyrosine kinase activity.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is an inhibitor of a non-receptor tyrosine kinase involved in growth factor signaling pathway(s). In some embodiments, the inhibitor is an antagonist of JAK2 tyrosine kinase activity, such as tyrphostin AG490 (2-cyano-3-(3,4-dihydroxyphenyl)-N-(benzyl)-2-propenamide). In other embodiments, the inhibitor is an antagonist of bcr-abl tyrosine kinase activity, such as GLEEVEC™ imatinib mesylate.

In another aspect, the invention features a method of treating cancer by administering to a cancer patient effective amounts of IP-10 and pirfenidone or pirfenidone analog and co-administering to the cancer patient an effective amount of at least one additional antineoplastic drug that is a serine/threonine kinase inhibitor. In some embodiments, the serine/threonine kinase inhibitor is a receptor serine/threonine kinase inhibitor, such as antagonists of TGF-β receptor serine/threonine kinase activity. In other embodiments, the serine/threonine kinase inhibitor is a non-receptor serine/threonine kinase inhibitor, such as antagonists of the serine/threonine kinase activity of the MAP kinases, protein kinase C (PKC), protein kinase A (PKA), or the cyclin-dependent kinases (CDKs).

In another aspect, the invention features a method of treating cancer by administering to a cancer patient effective amounts of IP-10 and pirfenidone or pirfenidone analog and co-administering to the cancer patient an effective amount of at least one additional antineoplastic drug that is an inhibitor of one or more kinases involved in cell cycle regulation. In some embodiments, the inhibitor is an antagonist of CDK2 activation, such as tryphostin AG490 (2-cyano-3-(3,4-dihydroxyphenyl)-N-(benzyl)-2-propenamide). In other embodiments, the inhibitor is an antagonist of CDK1/cyclin B activity, such as alsterpaullone. In still other embodiments, the inhibitor is an antagonist of CDK2 kinase activity, such as indirubin-3′-monoxime.

In another aspect, the invention features a method of treating cancer in a patient by co-administering to the patient effective amounts of IP-10, pirfenidone, a taxane, and a platinum complex. In some embodiments, the taxane is paclitaxel and the platinum complex is cisplatin or carboplatin.

In another aspect, the invention features a method of treating cancer in a patient by co-administering to the patient an effective amounts IP-10 and pirfenidone and an effective amount of at least one additional antineoplastic drug that is an a tumor-associated antigen antagonist, such as an antibody antagonist. In some embodiments involving the treatment of HER2-expressing tumors, the tumor-associated antigen antagonist is an anti-HER2 monoclonal antibody, such as HERCEPTIN™ trastuzumab. In some embodiments involving the treatment of CD20-expressing,tumors, such as B-cell lymphomas, the tumor-associated antigen antagonist is an anti-CD20 monoclonal antibody, such as RITUXAN™ rituximab.

In another aspect, the invention features a method of treating cancer in a patient by co-administering to the patient effective amounts of IP-10 and pirfenidone and an effective amount of at least one additional antineoplastic drug that is a tumor growth factor antagonist. In some embodiments, the tumor growth factor antagonist is an antagonist of epidermal growth factor (EGF), such as an anti-EGF monoclonal antibody. In other embodiments, the tumor growth factor antagonist is an antagonist of epidermal growth factor receptor erbB1 (EGFR), such as an anti-EGFR monoclonal antibody antagonist of EGFR activation or signal transduction.

In another aspect, the invention features a method of treating cancer in a patient by co-administering to the patient effective amounts of IP-10 and pirfenidone and an effective amount of at least one additional antineoplastic drug that is an Apo-2 ligand agonist

In another aspect, the invention features a method of treating cancer in a patient by co-administering to the patient effective amounts of IP-10 and pirfenidone and an effective amount of at least one additional antineoplastic drug that is an anti-angiogenic agent. In some embodiments, the anti-angiogenic agent is a vascular endothelial cell growth factor (VEGF) antagonist, such as an anti-VEGF monoclonal antibody, e.g. AVASTIN™ bevacizumab. In other embodiments, the anti-angiogenic agent is a retinoic acid receptor (RXR) ligand. In still other embodiments, the anti-angiogenic agent is a peroxisome proliferator-activated receptor (PPAR) gamma ligand.

In another aspect, the invention features any of the above-described methods of treating cancer in a patient, where the subject method further comprises co-administering to the patient effective amounts of a RXR ligand and a PPAR gamma ligand.

In another aspect, the invention features any of the above-described methods of treating cancer in a patient, where the subject method further comprises co-administering to the patient an effective amount of lometrexol.

In another aspect, the invention features a method of treating cancer in a patient by subjecting the patient to radiation therapy and by co-administering to the patient effective amounts of IP-10 and pirfendione or pirfenidone analog.

In another aspect, the invention features a method of treating cancer in a patient by subjecting the patient to surgical excision of part or all of a tumor mass carried by the patient and by co-administering to the patient effective amounts of IP-10 and pirfenidone or pirfenidone analog.

In another aspect, the invention features a method of treating cancer in a patient by subjecting the patient to radiation therapy and surgical excision of part or all of a tumor mass carried by the patient and by co-administering to the patient effective amounts of IP-10 and pirfenidone or pirfenidone analog.

In another aspect, the invention features any of the above-described methods of treating a cancer in a patient in which the patient receives (i) effective amounts of IP-10 and pirfenidone or pirfenidone analog and (ii) an effective amount of at least one additional drug that is an antineoplastic drug or biological response modifier, where the subject method further comprises subjecting the patient to radiation therapy.

In another aspect, the invention features any of the above-described methods of treating a cancer in a patient in which the patient receives (i) effective amounts of IP-10 and pirfenidone or pirfenidone analog and (ii) an effective amount of at least one additional drug that is an antineoplastic drug or biological response modifier, where the subject method further comprises subjecting the patient to surgical excision of part or all of a tumor mass carried by the patient.

In another aspect, the invention features any of the above-described methods of treating a cancer in a patient in which the patient receives (i) effective amounts of IP-10 and pirfenidone or pirfenidone analog and (ii) an effective amount of at least one additional drug that is an antineoplastic drug or biological response modifier, where the subject method further comprises subjecting the patient to radiation therapy and surgical excision of part or all of a tumor mass carried by the patient.

In another aspect, the invention features kits and articles of manufacture. In some embodiments, the kit or article of manufacture comprises: (a) a container comprising an amount of IP-10 that in combination with an amount of pirfenidone or a pirfenidone analog is effective for the treatment of a patient suffering from cancer; and (b) a label comprising printed instructions for the co-administration to the patient of the amount of IP-10 and the amount of pirfenidone or the pirfenidone analog.

In other embodiments, the kit or article of manufacture comprises: (a) a container comprising an amount of pirfenidone or a pirfenidone analog that in combination with an amount of IP-10 is effective for the treatment of a patient suffering from cancer; and (b) a label comprising printed instructions for the co-administration to the patient of the amount of pirfenidone or the pirfenidone analog and the amount of IP-10.

In still other embodiments, the kit or article of manufacture comprises: (a) a container comprising an amount of pirfenidone or a pirfenidone analog and an amount of IP-10 that in combination are effective for the treatment of a patient suffering from cancer; and (b) a label comprising printed instructions for the co-administration to the patient of the amount of pirfenidone or the pirfenidone analog and the amount of IP-10.

In additional embodiments, the kit or article of manufacture comprises: (a) a first container comprising an amount of pirfenidone or a pirfenidone analog; (b) a second container comprising an amount of IP-10 that in combination with the amount of pirfenidone or the pirfenidone analog is effective for the treatment of a patient suffering from cancer; and (c) a label comprising printed instructions for the co-administration to the patient of the amount of pirfenidone or the pirfenidone analog and the amount of IP-10.

The present invention further features diagnostic methods for determining the responsiveness of a given tumor to treatment with IP-10 and pirfenidone combination therapy.

Type I Interferon Receptor Agonist and Pirfenidone Combination Therapy

The present invention features a method of treating cancer, generally involving combination therapy with effective amounts of a Type I interferon receptor agonist and pirfenidone or a pirfenidone analog. The methods are useful as primary cancer therapy, or as adjuvant therapy. In some embodiments, the Type I interferon receptor agonist is an interferon-α (IFN-α). In other embodiments, the IFN-α is a PEGylated IFN-α.

In one aspect, the invention features a method of treating cancer by administering to a cancer patient effective amounts of a Type I interferon receptor agonist and pirfenidone or pirfenidone analog and co-administering to the cancer patient an effective amount of at least one additional drug that is a biological response modifier.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is a chemotherapeutic agent.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is an anti-angiogenic agent.

In another aspect, the invention features a method of treating cancer by administering to a cancer patient effective amounts of a Type I interferon receptor agonist and pirfenidone or pirfenidone analog and co-administering to the cancer patient an effective amount of IP-10. In some embodiments, the Type I interferon receptor agonist is an interferon-α (IFN-α). In other embodiments, the IFN-α is a PEGylated IFN-α.

In another aspect, the invention features a method of treating cancer by administering to a cancer patient effective amounts of a Type I interferon receptor agonist and pirfenidone or pirfenidone analog and co-administering to the cancer patient an effective amount of a Type II interferon receptor agonist. In some embodiments, the Type II interferon receptor agonist is an interferon-γ (IFN-γ).

In another aspect, the invention features a method of treating cancer by administering to a cancer patient effective amounts of a Type I interferon receptor agonist and pirfenidone or pirfenidone analog and co-administering to the patient effective amounts of IP-10 and a Type II interferon receptor agonist. In some embodiments, the Type I interferon receptor agonist is IFN-α and the Type II interferon receptor agonist is IFN-γ.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is an alkylating agent. In some embodiments, the alkylating agent is a nitrogen mustard. In other embodiments, the alkylating agent is an ethylenimine. In still other embodiments, the alkylating agent is an alkylsulfonate. In additional embodiments, the alkylating agent is a triazene. In further embodiments, the alkylating agent is a nitrosourea.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is an antimetabolite. In some embodiments, the antimetabolite is a folic acid analog, such as methotrexate. In other embodiments, the antimetabolite is a purine analog, such as mercaptopurine, thioguanine and axathioprine. In still other embodiments, the antimetabolite is a pyrimidine analog, such as 5FU, UFT, capecitabine, gemcitabine and cytarabine.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is a vinca alkyloid. In some embodiments, the vinca alkaloid is a taxane, such as paclitaxel. In other embodiments, the vinca alkaloid is a podophyllotoxin, such as etoposide, teniposide, ironotecan, and topotecan.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is an antineoplastic antibiotic. In some embodiments, the antineoplastic antibiotic is doxorubicin.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is a platinum complex. In some embodiments, the platinum complex is cisplatin. In other embodiments, the platinum complex is carboplatin.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is a tyrosine kinase inhibitor. In some embodiments, the tyrosine kinase inhibitor is a receptor tyrosine kinase (RTK) inhibitor, such as type I receptor tyrosine kinase inhibitors (e.g., inhibitors of epidermal growth factor receptors), type II receptor tyrosine kinase inhibitors (e.g., inhibitors of insulin receptor), type III receptor tyrosine kinase inhibitors (e.g., inhibitors of platelet-derived growth factor receptor), and type IV receptor tyrosine kinase inhibitors (e.g., fibroblast growth factor receptor). In other embodiments, the tyrosine kinase inhibitor is a non-receptor tyrosine kinase inhibitor, such as inhibitors of src kinases or janus kinases.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is an inhibitor of a receptor tyrosine kinase involved in growth factor signaling pathway(s). In some embodiments, the inhibitor is genistein. In other embodiments, the inhibitor is an epidermal growth factor receptor (EGFR) tyrosine kinase-specific antagonist, such as IRESSA™ gefitinib, TARCEVA™ erolotinib, or tyrphostin AG1478 (4-(3-chloroanilino)-6,7-dimethoxyquinazoline. In still other embodiments, the inhibitor is any indolinone antagonist of Flk-1/KDR (VEGF-R2) tyrosine kinase activity. In further embodiments, the inhibitor is any of the substituted 3-[(4,5,6,7-tetrahydro-1H-indol-2-yl)methylene]-1,3-dihydroindol-2-one antagonist of Flk-1/KDR (VEGF-R2), FGF-R1 or PDGF-R tyrosine kinase activity. In additional embodiments, the inhibitor is any substituted 3-[(3- or 4-carboxyethylpyrrol-2-yl)methylidenyl]indolin-2-one antagonist of Fit-1 (VEGF-R1), Flk-1/KDR (VEGF-R2), FGF-R1 or PDGF-R tyrosine kinase activity.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is an inhibitor of a non-receptor tyrosine kinase involved in growth factor signaling pathway(s). In some embodiments, the inhibitor is an antagonist of JAK2 tyrosine kinase activity, such as tyrphostin AG490 (2-cyano-3-(3,4-dihydroxyphenyl)-N-(benzyl)-2-propenamide). In other embodiments, the inhibitor is an antagonist of bcr-abl tyrosine kinase activity, such as GLEEVEC™ imatinib mesylate.

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is a serine/threonine kinase inhibitor. In some embodiments, the serine/threonine kinase inhibitor is a receptor serine/threonine kinase inhibitor, such as antagonists of TGF-β receptor serine/threonine kinase activity. In other embodiments, the serine/threonine kinase inhibitor is a non-receptor serine/threonine kinase inhibitor, such as antagonists of the serine/threonine kinase activity of the MAP kinases, protein kinase C (PKC), protein kinase A (PKA), or the cyclin-dependent kinases (CDKs).

In another aspect, the invention features a method of treating cancer by administering to a cancer patient effective amounts of a Type I interferon receptor agonist and pirfenidone or pirfenidone analog and co-administering to the cancer patient an effective amount of at least one additional antineoplastic drug that is an inhibitor of one or more kinases involved in cell cycle regulation. In some embodiments, the inhibitor is an antagonist of CDK2 activation, such as tryphostin AG490 (2-cyano-3-(3,4-dihydroxyphenyl)-N-(benzyl)-2-propenamide). In other embodiments, the inhibitor is an antagonist of CDK1/cyclin B activity, such as alsterpaullone. In still other embodiments, the inhibitor is an antagonist of CDK2 kinase activity, such as indirubin-3′-monoxime.

In another aspect, the invention features a method of treating cancer in a patient by co-administering to the patient effective amounts of a Type I interferon receptor agonist, pirfenidone, a taxane, and a platinum complex. In some embodiments, the taxane is paclitaxel and the platinum complex is cisplatin or carboplatin.

In another aspect, the invention features a method of treating cancer in a patient by co-administering to the patient an effective amounts a Type I interferon receptor agonist and pirfenidone and an effective amount of at least one additional antineoplastic drug that is an a tumor-associated antigen antagonist, such as an antibody antagonist In some embodiments involving the treatment of HER2-expressing tumors, the tumor-associated antigen antagonist is an anti-HER2 monoclonal antibody, such as HERCEPTIN™ trastuzumab. In some embodiments involving the treatment of CD20-expressing tumors, such as B-cell lymphomas, the tumor-associated antigen antagonist is an anti-CD20 monoclonal antibody, such as RITUXAN™ rituximab.

In another aspect, the invention features a method of treating cancer in a patient by co-administering to the patient effective amounts of a Type I interferon receptor agonist and pirfenidone and an effective amount of at least one additional antineoplastic drug that is a tumor growth factor antagonist. In some embodiments, the tumor growth factor antagonist is an antagonist of epidermal growth factor (EGF), such as an anti-EGF monoclonal antibody. In other embodiments, the tumor growth factor antagonist is an antagonist of epidermal growth factor receptor erbB1 (EGFR), such as an anti-EGFR monoclonal antibody antagonist of EGFR activation or signal transduction.

In another aspect, the invention features a method of treating cancer in a patient by co-administering to the patient effective amounts of a Type I interferon receptor agonist and pirfenidone and an effective amount of at least one additional antineoplastic drug that is an Apo-2 ligand agonist.

In another aspect, the invention features a method of treating cancer in a patient by co-administering to the patient effective amounts of a Type I interferon receptor agonist and pirfenidone and an effective amount of at least one additional antineoplastic drug that is an anti-angiogenic agent. In some embodiments, the anti-angiogenic agent is a vascular endothelial cell growth factor (VEGF) antagonist, such as an anti-VEGF monoclonal antibody, e.g. AVASTIN™ bevacizumab. In other embodiments, the anti-angiogenic agent is a retinoic acid receptor (RXR) ligand. In still other embodiments, the anti-angiogenic agent is a peroxisome proliferator-activated receptor (PPAR) gamma ligand.

In another aspect, the invention features any of the above-described methods of treating cancer in a patient, where the subject method further comprises co-administering to the patient effective amounts of a RXR ligand and a PPAR gamma ligand.

In another aspect, the invention features any of the above-described methods of treating cancer in a patient, where the subject method further comprises co-administering to the patient an effective amount of lometrexol.

In another aspect, the invention features a method of treating cancer in a patient by subjecting the patient to radiation therapy and by co-administering to the patient effective amounts of a Type I interferon receptor agonist and pirfendione or pirfenidone analog.

In another aspect, the invention features a method of treating cancer in a patient by subjecting the patient to surgical excision of part or all of a tumor mass carried by the patient and by co-administering to the patient effective amounts of a Type I interferon receptor agonist and pirfenidone or pirfenidone analog.

In another aspect, the invention features a method of treating cancer in a patient by subjecting the patient to radiation therapy and surgical excision of part or all of a tumor mass carried by the patient and by co-administering to the patient effective amounts of a Type I interferon receptor agonist and pirfenidone or pirfenidone analog.

In another aspect, the invention features any of the above-described methods of treating a cancer in a patient in which the patient receives (i) effective amounts of a Type I interferon receptor agonist and pirfenidone or pirfenidone analog and (ii) an effective amount of at least one additional drug that is an antineoplastic drug or biological response modifier, where the subject method further comprises subjecting the patient to radiation therapy.

In another aspect, the invention features any of the above-described methods of treating a cancer in a patient in which the patient receives (i) effective amounts of a Type I interferon receptor agonist and pirfenidone or pirfenidone analog and (ii) an effective amount of at least one additional drug that is an antineoplastic drug or biological response modifier, where the subject method further comprises subjecting the patient to surgical excision of part or all of a tumor mass carried by the patient.

In another aspect, the invention features any of the above-described methods of treating a cancer in a patient in which the patient receives (i) effective amounts of a Type I interferon receptor agonist and pirfenidone or pirfenidone analog and (ii) an effective amount of at least one additional drug that is an antineoplastic drug or biological response modifier, where the subject method further comprises subjecting the patient to radiation therapy and surgical excision of part or all of a tumor mass carried by the patient.

In another aspect, the invention features kits and articles of manufacture. In some embodiments, the kit or article of manufacture comprises: (a) a container comprising an amount of a Type I interferon receptor agonist that in combination with an amount of pirfenidone or a pirfenidone analog is effective for the treatment of a patient suffering from cancer; and (b) a label comprising printed instructions for the co-administration to the patient of the amount of the Type I interferon receptor agonist and the amount of pirfenidone or the pirfenidone analog.

In other embodiments, the kit or article of manufacture comprises: (a) a container comprising an amount of pirfenidone or a pirfenidone analog that in combination with an amount of a Type-I interferon receptor agonist is effective for the treatment of a patient suffering from cancer; and (b) a label comprising printed instructions for the co-administration to the patient of the amount of pirfenidone or the pirfenidone analog and the amount of the Type I interferon receptor agonist.

In still other embodiments, the kit or article of manufacture comprises: (a) a container comprising an amount of pirfenidone or a pirfenidone analog and an amount of a Type I interferon receptor agonist that in combination are effective for the treatment of a patient suffering from cancer; and (b) a label comprising printed instructions for the co-administration to the patient of the amount of pirfenidone or the pirfenidone analog and the amount of the Type I interferon receptor agonist.

In additional embodiments, the kit or article of manufacture comprises: (a) a first container comprising an amount of pirfenidone or a pirfenidone analog; (b) a second container comprising an amount of a Type I interferon receptor agonist that in combination with the amount of pirfenidone or the pirfenidone analog is effective for the treatment of a patient suffering from cancer; and (c) a label comprising printed instructions for the co-administration to the patient of the amount of pirfenidone or the pirfenidone analog and the amount of the Type I interferon receptor agonist.

The present invention further features diagnostic methods for determining the responsiveness of a given tumor to treatment with a Type I interferon receptor agonist and pirfenidone combination therapy.

Type I Interferon Receptor Agonist and IP-10 Combination Therapy

The present invention features a method of treating cancer, generally involving combination therapy with effective amounts of a Type I interferon receptor agonist and IP-10. The methods are useful as primary cancer therapy, or as adjuvant therapy. In some embodiments, the Type I interferon receptor agonist is an interferon-α (IFN-α). In other embodiments, the IFN-α is a PEGylated IFN-α.

In one aspect, the invention features a method of treating cancer by administering to a cancer patient effective amounts of a Type I interferon receptor agonist and IP-10 and co-administering to the cancer patient an effective amount of at least one additional drug that is a biological response modifier.

In another aspect, the invention features a method of treating cancer by administering to a cancer patient effective amounts of a Type I interferon receptor agonist and IP-10 and co-administering to the cancer patient an effective amount of at least one additional antineoplastic drug.

In another aspect, the invention features a method of treating cancer by administering to a cancer patient effective amounts of a Type I interferon receptor agonist and IP-10 and co-administering to the cancer patient an effective amount of at least one additional antineoplastic drug that is a chemotherapeutic agent.

In another aspect, the invention features a method of treating cancer by administering to a cancer patient effective amounts of a Type I interferon receptor agonist and IP-10 and co-administering to the cancer patient an effective amount of at least one additional antineoplastic drug that is an anti-angiogenic agent.

In another aspect, the invention features a method of treating cancer by administering to a cancer patient effective amounts of a Type I interferon receptor agonist and IP-10 and co-administering to the cancer patient an effective amount of pirfenidone or a pirfenidone analog. In some embodiments, the Type I interferon receptor agonist is an interferon-α (IFN-α). In other embodiments, the IFN-α is a PEGylated IFN-α.

In another aspect, the invention features a method of treating cancer by administering to a cancer patient effective amounts of a Type I interferon receptor agonist and IP-10 and co-administering to the cancer patient an effective amount of a Type II interferon receptor agonist. In some embodiments, the Type II interferon receptor agonist is an interferon-γ (IFN-γ).

In another aspect, the invention features a method of treating cancer by administering to a cancer patient effective amounts of a Type I interferon receptor agonist and IP-10 and co-administering to the patient effective amounts of pirfenidone or a pirfenidone analog and a Type II interferon receptor agonist. In some embodiments, the Type I interferon receptor agonist is IFN-α and the Type II interferon receptor agonist is IFN-γ.

In another aspect, the invention features a method of treating cancer by administering to a cancer patient effective amounts of a Type I interferon receptor agonist and IP-10 and co-administering to the cancer patient an effective amount of at least one additional antineoplastic drug that is an alkylating agent. In some embodiments, the alkylating agent is a nitrogen mustard. In other embodiments, the alkylating agent is an ethylenimine. In still other embodiments, the alkylating agent is an alkylsulfonate. In additional embodiments, tile alkylating agent is a triazene. In further embodiments, the alkylating agent is a nitrosourea

In another aspect, the invention features a method of treating cancer by administering to a cancer patient effective amounts of a Type I interferon receptor agonist and IP-10 and co-administering to the cancer patient an effective amount of at least one additional antineoplastic drug that is an antimetabolite. In some embodiments, the antimetabolite is a folic acid analog, such as methotrexate. In other embodiments, the antimetabolite is a purine analog, such as mercaptopurine, thioguanine and axathioprine. In still other embodiments, the antimetabolite is a pyrimidine analog, such as 5FU, UFT, capecitabine, gemcitabine and cytarabine.

In another aspect, the invention features a method of treating cancer by administering to a cancer patient effective amounts of a Type I interferon receptor agonist and IP-10 and co-administering to the cancer patient an effective amount of at least one additional antineoplastic drug that is a vinca alkyloid. In some embodiments, the vinca alkaloid is a taxane, such as paclitaxel. In other embodiments, the vinca alkaloid is a podophyllotoxin, such as etoposide, teniposide, ironotecan, and topotecan.

In another aspect, the invention features a method of treating cancer by administering to a cancer patient effective amounts of a Type I interferon receptor agonist and IP-10 and co-administering to the cancer patient an effective amount of at least one additional antineoplastic drug that is an antineoplastic antibiotic. In some embodiments, the antineoplastic antibiotic is doxorubicin.

In another aspect, the invention features a method of treating cancer by administering to a cancer patient effective amounts of a Type I interferon receptor agonist and IP-10 and co-administering to the cancer patient an effective amount of at least one additional antineoplastic drug that is a platinum complex. In some embodiments, the platinum complex is cisplatin. In other embodiments, the platinum complex is carboplatin.

In another aspect, the invention features a method of treating cancer by administering to a cancer patient effective amounts of a Type I interferon receptor agonist and IP-10 and co-administering to the cancer patient an effective amount of at least one additional antineoplastic drug that is a tyrosine kinase inhibitor. In some embodiments, the tyrosine kinase inhibitor is a receptor tyrosine kinase (RTK) inhibitor, such as type I receptor tyrosine kinase inhibitors (e.g., inhibitors of epidermal growth factor receptors), type II receptor tyrosine kinase inhibitors (e.g., inhibitors of insulin receptor), type III receptor tyrosine kinase inhibitors (e.g., inhibitors of platelet-derived growth factor receptor), and type IV receptor tyrosine kinase inhibitors (e.g., fibroblast growth factor receptor). In other embodiments, the tyrosine kinase inhibitor is a non-receptor tyrosine kinase inhibitor, such as inhibitors of src kinases or janus kinases.

In another aspect, the invention features a method of treating cancer by administering to a cancer patient effective amounts of a Type I interferon receptor agonist and IP-10 and co-administering to the cancer patient an effective amount of at least one additional antineoplastic drug that is an inhibitor of a receptor tyrosine kinase involved in growth factor signaling pathway(s). In some embodiments, the inhibitor is genistein. In other embodiments, the inhibitor is an epidermal growth factor receptor (EGFR) tyrosine kinase-specific antagonist, such as IRESSA™ gefitinib, TARCEVA™ erolotinib, or tyrphostin AG1478 (4-(3-chloroanilino)-6,7-dimethoxyquinazoline. In still other embodiments, the inhibitor is any indolinone antagonist of Flk-1/KDR (VEGF-R2) tyrosine kinase activity. In further embodiments, the inhibitor is any of the substituted 3-[(4,5,6,7-tetrahydro-1H-indol-2-yl)methylene]-1,3-dihydroindol-2-one antagonist of Flk-1/KDR (VEGF-R2), FGF-R1 or PDGF-R tyrosine kinase activity. In additional embodiments, the inhibitor is any substituted 3-[(3- or 4-carboxyethylpyrrol-2-yl)methylidenyl]indolin-2-one antagonist of Flt-1 (VEGF-R1), Flk-1/KDR (VEGF-R2), FGF-R1 or PDGF-R tyrosine kinase activity.

In another aspect, the invention features a method of treating cancer by administering to a cancer patient effective amounts of a Type I interferon receptor agonist and IP-10 and co-administering to the cancer patient an effective amount of at least one additional antineoplastic drug that is an inhibitor of a non-receptor tyrosine kinase involved in growth factor signaling pathway(s). In some embodiments, the inhibitor is an antagonist of JAK2 tyrosine kinase activity, such as tyrphostin AG490 (2-cyano-3-(3,4-dihydroxyphenyl)-N-(benzyl)-2-propenamide). In other embodiments, the inhibitor is an antagonist of bcr-abl tyrosine kinase activity, such as GLEEVEC™ imatinib mesylate.

In another aspect, the invention features a method of treating cancer by administering to a cancer patient effective amounts of a Type I interferon receptor agonist and IP-10 and co-administering to the cancer patient an effective amount of at least one additional antineoplastic drug that is a serine/threonine kinase inhibitor. In some embodiments, the serine/threonine kinase inhibitor is a receptor serine/threonine kinase inhibitor, such as antagonists of TGF-β receptor serine/threonine kinase activity. In other embodiments, the serine/threonine kinase inhibitor is a non-receptor serine/threonine kinase inhibitor, such as antagonists of the serine/threonine kinase activity of the MAP kinases, protein kinase C (PKC), protein kinase A (PKA), or the cyclin-dependent kinases (CDKs).

In another aspect, the invention features a method of treating cancer by administering to a cancer patient effective amounts of a Type I interferon receptor agonist and IP-10 and co-administering to the cancer patient an effective amount of at least one additional antineoplastic drug that is an inhibitor of one or more kinase involved in cell cycle regulation. In some embodiments, the inhibitor is an antagonist of CDK2 activation, such as tryphostin AG490 (2-cyano-3-(3,4-dihydroxyphenyl)-N-(benzyl)-2-propenamide). In other embodiments, the inhibitor is an antagonist of CDK1/cyclin B activity, such as alsterpaullone. In still other embodiments, the inhibitor is an antagonist of CDK kidnase activity, such as indirubin-3′-monoxime.

In another aspect, the invention features a method of treating cancer in a patient by co-administering to the patient effective amounts of a Type I interferon receptor agonist, IP-10, a taxane, and a platinum complex. In some embodiments, the taxane is paclitaxel and the platinum complex is cisplatin or carboplatin.

In another aspect, the invention features a method of treating cancer in a patient by co-administering to the patient an effective amounts a Type I interferon receptor agonist and IP-10 and an effective amount of at least one additional antineoplastic drug that is an a tumor-associated antigen antagonist, such as an antibody antagonist. In some embodiments involving the treatment of HER2-expressing tumors, the tumor-associated antigen antagonist is an anti-HER2 monoclonal antibody, such as HERCEPTIN™ trastuzumab. In some embodiments involving the treatment of CD20-expressing tumors, such as B-cell lymphomas, the tumor-associated antigen antagonist is an anti-CD20 monoclonal antibody, such as RITUXAN™ rituximab.

In another aspect, the invention features a method of treating cancer in a patient by co-administering to the patient effective amounts of a Type I interferon receptor agonist and IP-10 and an effective amount of at least one additional antineoplastic drug that is a tumor growth factor antagonist. In some embodiments, the tumor growth factor antagonist is an antagonist of epidermal growth factor (EGF), such as an anti-EGF monoclonal antibody. In other embodiments, the tumor growth factor antagonist is an antagonist of epidermal growth factor receptor erbB1 (EGFR), such as an anti-EGFR monoclonal antibody antagonist of EGFR activation or signal transduction.

In another aspect, the invention features a method of treating cancer in a patient by co-administering to the patient effective amounts of a Type I interferon receptor agonist and IP-10 and an effective amount of at least one additional antineoplastic drug that is an Apo-2 ligand agonist.

In another aspect, the invention features a method of treating cancer in a patient by co-administering to the patient effective amounts of a Type I interferon receptor agonist and IP-10 and an effective amount of at least one additional antineoplastic drug that is an anti-angiogenic agent. In some embodiments, the anti-angiogenic agent is a vascular endothelial cell growth factor (VEGF) antagonist, such as an anti-VEGF monoclonal antibody, e.g. AVASTIN™ bevacizumab. In other embodiments, the anti-angiogenic agent is a retinoic acid receptor (RXR) ligand. In still other embodiments, the anti-angiogenic agent is a peroxisome proliferator-activated receptor (PPAR) gamma ligand.

In another aspect, the invention features any of the above-described methods of treating cancer in a patient, where the subject method further comprises co-administering to the patient effective amounts of a RXR ligand and a PPAR gamma ligand.

In another aspect, the invention features any of the above-described methods of treating cancer in a patient, where the subject method further comprises co-administering to the patient an effective amount of lometrexol.

In another aspect, the invention features a method of treating cancer in a patient by subjecting the patient to radiation therapy and by co-administering to the patient effective amounts of a Type I interferon receptor agonist and IP-10.

In another aspect, the invention features a method of treating cancer in a patient by subjecting the patient to surgical excision of part or all of a tumor mass carried by the patient and by co-administering to the patient effective amounts of a Type I interferon receptor agonist and IP-10.

In another aspect, the invention features a method of treating cancer in a patient by subjecting the patient to radiation therapy and surgical excision of part or all of a tumor mass carried by the patient and by co-administering to the patient effective amounts of a Type I interferon receptor agonist and IP-10.

In another aspect, the invention features any of the above-described methods of treating a cancer in a patient in which the patient receives (i) effective amounts of a Type I interferon receptor agonist and IP-10 and (ii) an effective amount of at least one additional drug that is an antineoplastic drug or biological response modifier, where the subject method further comprises subjecting the patient to radiation therapy.

In another aspect, the invention features any of the above-described methods of treating a cancer in a patient in which the patient receives (i) effective amounts of a Type I interferon receptor agonist and IP-10 and (ii) an effective amount of at least one additional drug that is an antineoplastic drug or biological response modifier, where the subject method further comprises subjecting the patient to surgical excision of part or all of a tumor mass carried by the patient

In another aspect, the invention features any of the above-described methods of treating a cancer in a patient in which the patient receives (i) effective amounts of a Type I interferon receptor agonist and IP-10 and (ii) an effective amount of at least one additional drug that is an antineoplastic drug or biological response modifier, where the subject method further comprises subjecting the patient to radiation therapy and surgical excision of part or all of a tumor mass carried by the patient.

In another aspect, the invention features kits and articles of manufacture. In some embodiments, the kit or article of manufacture comprises: (a) a container comprising an amount of a Type I interferon receptor agonist that in combination with an amount of IP-10 is effective for the treatment of a patient suffering from cancer; and (b) a label comprising printed instructions for the co-administration to the patient of the amount of IP-10 and the amount of the Type I interferon receptor agonist.

In other embodiments, the kit or article of manufacture comprises: (a) a container comprising an amount of IP-10 that in combination with an amount of a Type I interferon receptor agonist is effective for the treatment of a patient suffering from cancer; and (b) a label comprising printed instructions for the co-administration to the patient of the amount of IP-10 and the amount of the Type I interferon receptor agonist.

In still other embodiments, the kit or article of manufacture comprises: (a) a container comprising an amount of IP-10 and an amount of a Type I interferon receptor agonist that in combination are effective for the treatment of a patient suffering from cancer; and (b) a label comprising printed instructions for the co-administration to the patient of the amount of IP-10 and the amount of the Type I interferon receptor agonist.

In additional embodiments, the kit or article of manufacture comprises: (a) a first container comprising an amount of IP-10; (b) a second container comprising an amount of a Type I interferon receptor agonist that in combination with the amount of IP-10 is effective for the treatment of a patient suffering from cancer; and (c) a label comprising printed instructions for the co-administration to the patient of the amount of IP-10 and the amount of the Type I interferon receptor agonist.

The present invention further features diagnostic methods for determining the responsiveness of a given tumor to treatment with a Type I interferon receptor agonist and IP-10 combination therapy.

Pirfenidone and Additional Anti-Cancer Therapeutic Agent Combination Therapy

The present invention features a method of treating cancer, generally involving therapy with an effective amount of pirfenidone or a pirfenidone analog in combination with another treatment for cancer. The methods are useful as primary cancer therapy, or as adjuvant therapy.

In one aspect, the invention features a method of treating cancer by administering to a cancer patient an effective amount of pirfenidone or pirfenidone analog and co-administering to the cancer patient an effective amount of at least one additional drug that is a biological response modifier.

In another aspect, the invention features a method of treating cancer by administering to a cancer patient an effective amount of pirfenidone or pirfenidone analog and co-administering to the cancer patient an effective amount of at least one additional antineoplastic drug.

In another aspect, the invention features a method of treating cancer by administering to a cancer patient an effective amount of pirfenidone or pirfenidone analog and co-administering to the cancer patient an effective amount of at least one additional antineoplastic drug that is a chemotherapeutic agent.

In another aspect, the invention features a method of treating cancer by administering to a cancer patient an effective amount of pirfenidone or pirfenidone analog and co-administering to the cancer patient an effective amount of at least one additional antineoplastic drug that is an anti-angiogenic agent.

In another aspect, the invention features a method of treating cancer by administering to a cancer patient an effective amount of pirfenidone or pirfenidone analog and co-administering to the cancer patient an effective amount of a Type I interferon receptor agonist. In some embodiments, the Type I interferon receptor agonist is an interferon-α (IFN-α). In other embodiments, the IFN-α is a PEGylated IFN-α.

In another aspect, the invention features a method of treating cancer by administering to a cancer patient an effective amount of pirfenidone or pirfenidone analog and co-administering to the cancer patient an effective amount of a Type II interferon receptor agonist. In some embodiments, the Type II interferon receptor agonist is an interferon-γ (IFN-γ).

In another aspect, the invention features a method of treating cancer by administering to a cancer patient an effective amount of pirfenidone or pirfenidone analog and co-administering to the cancer patient an effective amount of a Type III interferon receptor agonist. In some embodiments, the Type III interferon receptor agonist is IL-28A, IL-28B, or IL-29.

In another aspect, the invention features a method of treating cancer by administering to a cancer patient an effective amount of pirfenidone or pirfenidone analog and co-administering to the patient effective amounts of a Type I interferon receptor agonist and a Type II interferon receptor agonist In some embodiments, the Type I interferon receptor agonist is IFN-α and the Type II interferon receptor agonist is IFN-γ.

In another aspect, the invention features a method of treating cancer by administering to a cancer patient an effective amount of pirfenidone or pirfenidone analog and co-administering to the cancer patient an effective amount of at least one additional antineoplastic drug that is an alkylating agent. In some embodiments, the alkylating agent is a nitrogen mustard. In other embodiments, the alkylating agent is an ethylenimine. In still other embodiments, the alkylating agent is an alkylsulfonate. In additional embodiments, the alkylating agent is a triazene. In further embodiments, the alkylating agent is a nitrosourea.

In another aspect, the invention features a method of treating cancer by administering to a cancer patient an effective amount of pirfenidone or pirfenidone analog and co-administering to the cancer patient an effective amount of at least one additional antineoplastic drug that is an antimetabolite. In some embodiments, the antimetabolite is a folic acid analog, such as methotrexate. In other embodiments, the antimetabolite is a purine analog, such as mercaptopurine, thioguanine and axathioprine. In still other embodiments, the antimetabolite is a pyrimidine analog, such as 5FU, UFT, capecitabine, gemcitabine and cytarabine.

In another aspect, the invention features a method of treating cancer by administering to a cancer patient an effective amount of pirfenidone or pirfenidone analog and co-administering to the cancer patient an effective amount of at least one additional antineoplastic drug that is a vinca alkyloid. In some embodiments, the vinca alkaloid is a taxane, such as paclitaxel. In other embodiments, the vinca alkaloid is a podophyllotoxin, such as etoposide, teniposide, ironotecan, and topotecan.

In another aspect, the invention features a method of treating cancer by administering to a cancer patient an effective amount of pirfenidone or pirfenidone analog and co-administering to the cancer patient an effective amount of at least one additional antineoplastic drug that is an antineoplastic antibiotic. In some embodiments, the antineoplastic antibiotic is doxorubicin.

In another aspect, the invention features a method of treating cancer by administering to a cancer patient an effective amount of pirfenidone or pirfenidone analog and co-administering to the cancer patient an effective amount of at least one additional antineoplastic drug that is a platinum complex. In some embodiments, the platinum complex is cisplatin. In other embodiments, the platinum complex is carboplatin.

In another aspect, the invention features a method of treating cancer by administering to a cancer patient an effective amount of pirfenidone or pirfenidone analog and co-administering to the cancer patient an effective amount of at least one additional antineoplastic drug that is a tyrosine kinase inhibitor. In some embodiments, the tyrosine kinase inhibitor is a receptor tyrosine kinase (RTK) inhibitor, such as type I receptor tyrosine kinase inhibitors (e.g., inhibitors of epidermal growth factor receptors), type II receptor tyrosine kinase inhibitors (e.g., inhibitors of insulin receptor), type III receptor tyrosine kinase inhibitors (e.g., inhibitors of platelet-derived growth factor receptor), and type IV receptor tyrosine kinase inhibitors (e.g., fibroblast growth factor receptor). In other embodiments, the tyrosine kinase inhibitor is a non-receptor tyrosine kinase inhibitor, such as inhibitors of src kinases or janus kinases.

In another aspect, the invention features a method of treating cancer by administering to a cancer patient an effective amount of pirfenidone or pirfenidone analog and co-administering to the cancer patient an effective amount of at least one additional antineoplastic drug that is an inhibitor of a receptor tyrosine kinase involved in growth factor signaling pathway(s). In some embodiments, the inhibitor is genistein. In other embodiments, the inhibitor is an epidermal growth factor receptor (EGFR) tyrosine kinase-specific antagonist, such as IRESSA™ gefitinib, TARCEVA™ erolotinib, or tyrphostin AG1478 (4-(3-chloroanilino)-6,7-dimethoxyquinazoline. In still other embodiments, the inhibitor is any indolinone antagonist of Flk-1/KDR (VEGF-R2) tyrosine kinase activity. In further embodiments, the inhibitor is any of the substituted 3-[(4,5,6,7-tetrahydro-1H-indol-2-yl)methylene]-1,3-dihydroindol-2-one antagonist of Flk-1/KDR (VEGF-R2), FGF-R1 or PDGF-R tyrosine kinase activity. In additional embodiments, the inhibitor is any substituted 3-[(3- or 4-carboxyethylpyrrol-2-yl)methylidenyl]indolin-2-one antagonist of Flt-1 (VEGF-R1), Flk-1/KDR (VEGF-R2), FGF-R1 or PDGF-R tyrosine kinase activity.

In another aspect, the invention features a method of treating cancer by administering to a cancer patient an effective amount of pirfenidone or pirfenidone analog and co-administering to the cancer patient an effective amount of at least one additional antineoplastic drug that is an inhibitor of a non-receptor tyrosine kinase involved in growth factor signaling pathway(s). In some embodiments, the inhibitor is an antagonist of JAK2 tyrosine kinase activity, such as tyrphostin AG490 (2-cyano-3-(3,4-dihydroxyphenyl)-N-(benzyl)-2-propenamide). In other embodiments, the inhibitor is an antagonist of bcr-abl tyrosine kinase activity, such as GLEEVEC™ imatinib mesylate.

In another aspect, the invention features a method of treating cancer by administering to a cancer patient an effective amount of pirfenidone or pirfenidone analog and co-administering to the cancer patient an effective amount of at least one additional antineoplastic drug that is a serine/threonine kinase inhibitor. In some embodiments, the serine/threonine kinase inhibitor is a receptor serine/threonine kinase inhibitor, such as antagonists of TGF-β receptor serine/threonine kinase activity. In other embodiments, the serine/threonine kinase inhibitor is a non-receptor serine/threonine kinase inhibitor, such as antagonists of the serine/threonine kinase activity of the MAP kinases, protein kinase C (PKC), protein kinase A (PKA), or the cyclin-dependent kinases (CDKs).

In another aspect, the invention features a method of treating cancer by administering to a cancer patient an effective amount of pirfenidone or pirfenidone analog and co-administering to the cancer patient an effective amount of at least one additional antineoplastic drug that is an inhibitor of one or more kinases involved in cell cycle regulation. In some embodiments, the inhibitor is an antagonist of CDK2 activation, such as tryphostin AG490 (2-cyano-3-(3,4-dihydroxyphenyl)-N-(benzyl)-2-propenamide). In other embodiments, the inhibitor is an antagonist of CDK1/cyclin B activity, such as alsterpaullone. In still other embodiments, the inhibitor is an antagonist of CDK2 kinase activity, such as indirubin-3′-monoxime.

In another aspect, the invention features a method of treating cancer in a patient by co-administering to the patient an effective amounts of pirfenidone, a taxane, and a platinum complex. In some embodiments, the taxane is paclitaxel and the platinum complex is cisplatin or carboplatin.

In another aspect, the invention features a method of treating cancer in a patient by co-administering to the patient an effective amount of pirfenidone and an effective amount of at least one additional antineoplastic drug that is a tumor-associated antigen antagonist, such as an antibody antagonist. In some embodiments involving the treatment of HER2-expressing tumors, the tumor-associated antigen antagonist is an anti-HER2 monoclonal antibody, such as HERCEPTIN™ trastuzumab. In some embodiments involving the treatment of CD20-expressing tumors, such as B-cell lymphomas, the tumor-associated antigen antagonist is an anti-CD20 monoclonal antibody, such as RITUXAN™ rituximab.

In another aspect, the invention features a method of treating cancer in a patient by co-administering to the patient an effective amount of pirfenidone and an effective amount of at least one additional antineoplastic drug that is a tumor growth factor antagonist. In some embodiments, the tumor growth factor antagonist is an antagonist of epidermal growth factor (EGF), such as an anti-EGF monoclonal antibody. In other embodiments, the tumor growth factor antagonist is an antagonist of epidermal growth factor receptor erbB1 (EGFR), such as an anti-EGFR monoclonal antibody antagonist of EGFR activation or signal transduction.

In another aspect, the invention features a method of treating cancer in a patient by co-administering to the patient an effective amount of pirfenidone and an effective amount of at least one additional antineoplastic drug that is an Apo-2 ligand agonist.

In another aspect, the invention features a method of treating cancer in a patient by co-administering to the patient an effective amount of pirfenidone and an effective amount of at least one additional antineoplastic drug that is an anti-angiogenic agent. In some embodiments, the anti-angiogenic agent is a vascular endothelial cell growth factor (VEGF) antagonist, such as an anti-VEGF monoclonal antibody, e.g. AVASTIN™ bevacizumab. In other embodiments, the anti-angiogenic agent is an antagonist of VEGF-R1, such as an anti-VEGF-R1 monoclonal antibody. In other embodiments, the anti-angiogenic agent is an antagonist of VEGF-R2, such as an anti-VEGF-R2 monoclonal antibody. In other embodiments, the anti-angiogenic agent is an antagonist of basic fibroblast growth factor (bFGF), such as an anti-bFGF monoclonal antibody. In other embodiments, the anti-angiogenic agent is an antagonist of bFGF receptor, such as an anti-bFGF receptor monoclonal antibody. In other embodiments, the anti-angiogenic agent is an antagonist of TGF-β, such as an anti-TGF-β monoclonal antibody. In other embodiments, the anti-angiogenic agent is an antagonist of TGF-β receptor, such as an anti-TGF-β receptor monoclonal antibody. In other embodiments, the anti-angiogenic agent is a retinoic acid receptor (RXR) ligand. In still other embodiments, the anti-angiogenic agent is a peroxisome proliferator-activated receptor (PPAR) gamma ligand.

In another aspect, the invention features a method of treating cancer in a patient comprising co-administering to the patient an effective amount of pirfenidone or pirfenidone analog and effective amounts of a RXR ligand and a PPAR gamma ligand.

In another aspect, the invention features a method of treating cancer in a patient comprising co-administering to the patient an effective amount of pirfenidone or pirfenidone analog and an effective amount of lometrexol.

In another aspect, the invention features a method of treating cancer in a patient by subjecting the patient to radiation therapy and by co-administering to the patient an effective amount of pirfendione or pirfenidone analog.

In another aspect, the invention features a method of treating cancer in a patient by subjecting the patient to surgical excision of part or all of a tumor mass carried by the patient and by co-administering to the patient an effective amount of pirfenidone or pirfenidone analog.

In another aspect, the invention features a method of treating cancer in a patient by subjecting the patient to radiation therapy and surgical excision of part or all of a tumor mass carried by the patient and by co-administering to the patient an effective amount of pirfenidone or pirfenidone analog.

In another aspect, the invention features any of the above-described methods of treating a cancer in a patient in which the patient receives (i) an effective amount of pirfenidone or pirfenidone analog and (ii) an effective amount of at least one additional drug that is an antineoplastic drug or biological response modifier, where the subject method further comprises subjecting the patient to radiation therapy.

In another aspect, the invention features any of the above-described methods of treating a cancer in a patient in which the patient receives (i) an effective amount of pirfenidone or pirfenidone analog and (ii) an effective amount of at least one additional drug that is an antineoplastic drug or biological response modifier, where the subject method further comprises subjecting the patient to surgical excision of part or all of a tumor mass carried by the patient.

In another aspect, the invention features any of the above-described methods of treating a cancer in a patient in which the patient receives (i) an effective amount of pirfenidone or pirfenidone analog and (ii) an effective amount of at least one additional drug that is an antineoplastic drug or biological response modifier, where the subject method further comprises subjecting the patient to radiation therapy and surgical excision of part or all of a tumor mass carried by the patient.

In another aspect, the invention features kits and articles of manufacture. In some embodiments, the kit or article of manufacture comprises: (a) a container comprising an amount of pirfenidone or a pirfenidone analog effective for the treatment of a patient suffering from cancer; and (b) a label comprising printed instructions for the treatment of the patient with the amount of pirfenidone or the pirfenidone analog in combination with another therapy for cancer.

The present invention further features diagnostic methods for determining the responsiveness of a given tumor to combination therapy with pirfenidone or pirfenidone analog and an additional antineoplastic drug or biological response modifier.

### BRIEF DESCRIPTION OF THE DRAWINGS

FIG. 1 depicts antiproliferative effects of various amounts of pirfenidone in combination with 10 ng/ml IP-10 on the CAOV-3 cell line.

FIG. 2 depicts antiproliferative effects of various amounts of IP-10 in combination with 30 μg/ml pirfenidone on the CAOV-3 cell line.

FIG. 3 depicts antiproliferative effects of various amounts of INFERGEN® in combination with 30 μg/ml pirfenidone on the OVCAR cell line.

FIG. 4 depicts antiproliferative effects of various amounts of pirfenidone in combination with 2 ng/ml INFERGEN® on the CAOV-3 cell line.

FIG. 5 depicts antiproliferative effects of various amounts of INFERGEN® in combination with 30 μg/ml pirfenidone on the CAOV-3 cell line.

FIG. 6 depicts antiproliferative effects of various amounts of IP-10 in combination with 2 ng/ml INFERGEN® on the CAOV-3 cell line.

FIG. 7 depicts antiproliferative effects of various amounts of INFERGEN® in combination with 10 ng/ml IP-10 on the CAOV-3 cell line.

FIG. 8 depicts antiproliferative effects of various amounts of INFERGEN® in combination with 10 ng/ml IP-10 on OVCAR cells.

### DEFINITIONS

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. Cancerous cells can be benign or malignant.

A “specific pirfenidone analog,” and all grammatical variants thereof, refers to, and is limited to, each and every pirfenidone analog shown in Table 1.

By “individual” or “host” or “subject” or “patient” is meant any mammalian subject for whom diagnosis, treatment, or therapy is desired, particularly humans. Other subjects may include cattle, dogs, cats, guinea pigs, rabbits, rats, mice, horses, and so on.

The term “dosing event” as used herein refers to administration of an agent (e.g., an antineoplastic agent or other active agent) to a patient in need thereof, which event may encompass one or more releases of an agent (e.g., an antineoplastic agent or other active agent) from a drug dispensing device. Thus, the term “dosing event,” as used herein, includes, but is not limited to, installation of a continuous delivery device (e.g., a pump or other controlled release injectable system); and a single subcutaneous injection followed by installation of a continuous delivery system.

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, tuning of release) of a drug or other desired substance contained therein is controlled by or determined by the device itself and not substantially influenced by the environment of use, or releasing at a rate that is reproducible within the environment of use.

By “substantially continuous” as used in, for example, the context of “substantially continuous infusion” or “substantially continuous delivery” is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

The term “chemotherapeutic agent” or “chemotherapeutic” (or “chemotherapy”, in the case of treatment with a chemotherapeutic agent) is meant to encompass any non-proteinaceous (i.e., non-peptidic) chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33: 183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubincin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g. paclitaxel (TAXOL®, Bristol Meyers Squibb Oncology, Princeton, N.J.) and docetaxel (TAXOTERE®, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluromethylornithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids or derivatives of any of the above.

The term “antineoplastic” agent, drug or compound is meant to refer to any agent, including any chemotherapeutic agent, biological response modifier (including without limitation (i) proteinaceous, i.e. peptidic, molecules capable of elaborating or altering biological responses and (ii) non-proteinaceous, i.e. non-peptidic, molecules capable of elaborating or altering biological responses), cytotoxic agent, or cytostatic agent, that reduces proliferation of a neoplastic cell.

The term “biological response modifier” refers to any proteinaceous (i.e., peptidic) molecule or any non-proteinaceous (i.e., non-peptidic) molecule capable of elaborating or altering a biological response relevant to the treatment of cancer; Examples of biological response modifiers include antagonists of tumor-associated antigens, such as anti-tumor antigen antibodies, antagonists of cellular receptors capable of inducing cell proliferation, agonists of cellular receptors capable of inducing apoptosis, such as Apo-2 ligands, Type I interferon receptor agonists, such as interferon-α molecules and interferon-β molecules, Type II interferon receptor agonists, such as interferon-γ molecules, Type III interferon receptor agonists, such as IL-28A, IL-28B, and IL-29, antagonists of inflammatory cytokines, including tumor necrosis factor (TNF) antagonists, such as anti-TNF antibodies (e.g. REMICADE™ anti-TNF monoclonal antibody) and soluble TNF receptor (e.g. ENBREL™ TNF receptor-Ig immunoadhesin), growth factor cytokines, such as hematopoietic cytokines, including erythropoietins, such as EPOGEN™ epoetin-alfa, granulocyte colony stimulating factors (G-CSFs), such as NEUPOGEN™ filgrastim, granulocyte-macrophage colony stimulating factors (GM-CSFs), and thrombopoietins, lymphocyte growth factor cytokines, such as interleukin-2, and antagonists of growth-factor cytokines, including antagonists of angiogenic factors, e.g. vascular endothelial cell growth factor (VEGF) antagonists, such as AVASTIN™ bevacizumab (anti-VEGF monoclonal antibody).

As used herein, the term “a Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “a Type II interferon receptor agonist” refers to any naturally-occurring or non-naturally-occurring ligand of a human Type II interferon receptor which binds to and causes signal transduction via the receptor. Type II interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a dose” includes a plurality of such doses and reference to “the IP-10 polypeptide” includes reference to one or more IP-10 polypeptides and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating cancer, generally involving administering to an individual having a cancer a therapeutically effective amount of IP-10 in combination with a therapeutically effective amount of pirfenidone or a pirfenidone analog. The invention is based in part on the observation that the proliferation of an ovarian cancer cell line, CAOV-3, was reduced when cultured in vitro in the presence of 10 ng/ml IP-10 and pirfenidone. The methods are useful as primary therapy, or as adjuvant therapy, e.g., in combination with a standard cancer treatment.

The present invention further provides methods of determining the susceptibility of a cancerous cell to treatment with IP-10 and pirfenidone combination therapy. The methods have prognostic utility, and in some embodiments are used in conjunction with a treatment method of the invention.

The present invention further provides methods of treating cancer, generally involving administering to an individual having a cancer a therapeutically effective amount of a Type I interferon receptor agonist in combination with a therapeutically effective amount of pirfenidone or a pirfenidone analog. The invention is based in part on the observation that the proliferation of an ovarian cancer cell line, CAOV-3, was reduced when cultured in vitro in the presence of 1.56 ng/ml INFERGEN® and 30 μg/ml pirfenidone. The methods are useful as primary therapy, or as adjuvant therapy, e.g., in combination with a standard cancer treatment.

The present invention further provides methods of determining the susceptibility of a cancerous cell to treatment with a Type I interferon receptor agonist and pirfenidone combination therapy. The methods have prognostic utility, and in some embodiments are used in conjunction with a treatment method of the invention.

The present invention further provides methods of treating cancer, generally involving administering to an individual having a cancer a therapeutically effective amount of a Type I receptor agonist in combination with a therapeutically effective amount of IP-10. The invention is based in part on the observation that the proliferation of an ovarian cancer cell line, CAOV-3, was reduced when cultured in vitro in the presence of 2 ng/ml INFERGEN® and 1.56 ng/ml IP-10. The methods are useful as primary therapy, or as adjuvant therapy, e.g., in combination with a standard cancer treatment.

The present invention further provides methods of determining the susceptibility of a cancerous cell to treatment with a Type I interferon receptor agonist and IP-10 combination therapy. The methods have prognostic utility, and in some embodiments are used in conjunction with a treatment method of the invention.

The present invention further provides methods of treating cancer, generally involving administering to an individual having a cancer a therapeutically effective amount of pirfenidone or a pirfenidone analog, in combination with any other therapy for cancer. In the methods of the invention, the therapy with pirfenidone or a pirfenidone analog can be used as a primary therapy, or as an adjuvant, in combination with another therapy for cancer.

The present invention further provides methods of determining the susceptibility of a cancerous cell to treatment with pirfenidone in combination with another antineoplastic agent or biological response modifier. The methods have prognostic utility, and in some embodiments are used in conjunction with a treatment method of the invention.

Methods of Treating Cancer

The present invention provides methods of treating cancer in an individual having a cancer. In some embodiments, methods generally involve administering a therapeutically effective amount of IP-10 and a therapeutically effective amount of pirfenidone. In some embodiments, the methods generally involve administering a therapeutically effective amount of a Type I interferon receptor agonist and a therapeutically effective amount of pirfenidone or a pirfenidone analog. In some embodiments, the Type I interferon receptor agonist is an IFN-α. In some embodiments, the methods generally involve administering a therapeutically effective amount of a Type I receptor agonist and a therapeutically effective amount of IP-10. In some embodiments, the Type I receptor agonist is an IFN-α. In some embodiments, the methods generally involve administering a therapeutically effective amount of pirfenidone, in combination with any other therapy for cancer.

IP-10 and Pirfenidone Combination Therapy

In some embodiments, methods generally involve administering a therapeutically effective amount of IP-10 and a therapeutically effective amount of pirfenidone.

The methods are effective to reduce a tumor load by at least about 5%, at least about 10%, at least about 20%, at least about 25%, at least about 50%, at least about 75%, at least about 85%, or at least about 90%, up to total eradication of the tumor, when compared to a suitable control. Thus, in these embodiments, “effective amounts” of IP-10 and pirfenidone are amounts of IP-10 and pirfenidone that are sufficient to reduce tumor load by at least about 5%, at least about 10%, at least about 20%, at least about 25%, at least about 50%, at least about 75%, at least about 85%, or at least about 90%, up to total eradication of the tumor, when compared to a suitable control. In an experimental aniimal system, a suitable control may be a genetically identical animal not treated with the IP-10 and pirfenidone combination therapy. In non-experimental systems, a suitable control may be the tumor load present before administering the IP-10 and pirfenidone combination therapy. Other suitable controls may be a placebo control.

Whether a tumor load has been decreased can be determined using any known method, including, but not limited to, measuring solid tumor mass; counting the number of tumor cells using cytological assays; fluorescence-activated cell sorting (e.g., using antibody specific for a tumor-associated antigen) to determine the number of cells bearing a given tumor antigen; computed tomography scanning, magnetic resonance imaging, and/or x-ray imaging of the tumor to estimate and/or monitor tumor size; measuring the amount of tumor-associated antigen in a biological sample, e.g., blood or serum; and the like.

The methods are effective to reduce the growth rate of a tumor by at least about 5%, at least about 10%, at least about 20%, at least about 25%, at least about 50%, at least about 75%, at least about 85%, or at least about 90%, up to total inhibition of growth of the tumor, when compared to a suitable control. Thus, in these embodiments, “effective amounts” of IP-10 and pirfenidone are amounts of IP-10 and pirfenidone that are sufficient to reduce tumor growth rate by at least about 5%, at least about 10%, at least about 20%, at least about 25%, at least about 50%, at least about 75%, at least about 85%, or at least about 90%, up to total inhibition of tumor growth, when compared to a suitable control. In an experimental animal system, a suitable control may be tumor growth rate in a genetically identical animal not treated with the IP-10 and pirfenidone combination therapy. In non-experimental systems, a suitable control may be the tumor load or tumor growth rate present before administering the IP-10 and pirfenidone combination therapy. Other suitable controls may be a placebo control.

Whether growth of a tumor is inhibited can be determined using any known method, including, but not limited to, an in vivo assay for tumor growth; an in vitro proliferation assay as described in the Example; a ³H-thymidine uptake assay; and the like.

The methods are useful for treating a wide variety of cancers, including carcinomas, sarcomas, leukemias, and lymphomas.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

In many embodiments, the effective amounts of IP-10 and pirfenidone (or a pirfenidone analog) are synergistic amounts. As used herein, a “synergistic combination” or a “synergistic amount” of IP-10 and pirfenidone or a pirfenidone analog is a combined dosage that is more effective in the therapeutic or prophylactic treatment of cancer than the incremental improvement in treatment outcome that could be predicted or expected from a merely additive combination of (i) the therapeutic or prophylactic benefit of IP-10 when administered at that same dosage as a monotherapy and (ii) the therapeutic or prophylactic benefit of pirfenidone or a pirfenidone analog when administered at the same dosage as a monotherapy.

In some embodiments of the invention, a selected amount of IP-10 and a selected amount of pirfenidone or a pirfenidone analog are effective when used in combination therapy for a disease, but the selected amount of IP-10 and/or the selected amount of pirfenidone or a pirfenidone analog is ineffective when used in monotherapy for the disease. Thus, the invention encompasses (1) regimens in which a selected amount of pirfenidone or a pirfenidone analog enhances the therapeutic benefit of a selected amount of IP-10 when used in combination therapy for a disease, where the selected amount of pirfenidone or a pirfenidone analog provides no therapeutic benefit when used in monotherapy for the disease (2) regimens in which a selected amount of IP-10 enhances the therapeutic benefit of a selected amount of pirfenidone or a pirfenidone analog when used in combination therapy for a disease, where the selected amount of IP-10 provides no therapeutic benefit when used in monotherapy for the disease and (3) regimens in which a selected amount of IP-10 and a selected amount of pirfenidone or a pifenidone analog provide a therapeutic benefit when used in combination therapy for a disease, where each of the selected amounts of IP-10 and pirfenidone or a pirfenidone analog, respectively, provides no therapeutic benefit when used in monotherapy for the disease. As used herein, a “synergistically effective amount” of IP-10 and pirfenidone or a pirfenidone analog, and its grammatical equivalents, shall be understood to include any regimen encompassed by any of (1)-(3) above.

Type I Interferon Receptor Agonist and Pirfenidone Combination Therapy

In some embodiments, the methods generally involve administering a therapeutically effective amount of a Type I interferon receptor agonist and a therapeutically effective amount of pirfenidone or a pirfenidone analog. In some embodiments, the Type I interferon receptor agonist is an IFN-α.

The methods are effective to reduce a tumor load by at least about 5%, at least about 10%, at least about 20%, at least about 25%, at least about 50%, at least about 75%, at least about 85%, or at least about 90%, up to total eradication of the tumor, when compared to a suitable control. Thus, in these embodiments, “effective amounts” of a Type I interferon receptor agonist and pirfenidone are amounts of a Type I interferon receptor agonist and pirfenidone that are sufficient to reduce tumor load by at least about 5%, at least about 10%, at least about 20%, at least about 25%, at least about 50%, at least about 75%, at least about 85%, or at least about 90%, up to total eradication of the tumor, when compared to a suitable control. In an experimental animal system, a suitable control may be a genetically identical animal not treated with the Type I interferon receptor agonist and pirfenidone combination therapy. In non-experimental systems, a suitable control may be the tumor load present before administering the Type I interferon receptor agonist and pirfenidone combination therapy. Other suitable controls may be a placebo control.

Whether a tumor load has been decreased can be determined using any known method, including, but not limited to, measuring solid tumor mass; counting the number of tumor cells using cytological assays; fluorescence-activated cell sorting (e.g., using antibody specific for a tumor-associated antigen) to determine the number of cells bearing a given tumor antigen; computed tomography scanning, magnetic resonance imaging, and/or x-ray imaging of the tumor to estimate and/or monitor tumor size; measuring the amount of tumor-associated antigen in a biological sample, e.g., blood; and the like.

The methods are effective to reduce the growth rate of a tumor by at least about 5%, at least about 10%, at least about 20%, at least about 25%, at least about 50%, at least about 75%, at least about 85%, or at least about 90%, up to total inhibition of growth of the tumor, when compared to a suitable control. Thus, in these embodiments, “effective amounts” of a Type I interferon receptor agonist and pirfenidone are amounts of a Type I interferon receptor agonist and pirfenidone that are sufficient to reduce tumor growth rate by at least about 5%, at least about 10%, at least about 20%, at least about 25%, at least about 50%, at least about 75%, at least about 85%, or at least about 90%, up to total inhibition of tumor growth, when compared to a suitable control. In an experimental animal system, a suitable control may be a genetically identical animal not treated with the Type I interferon receptor agonist and pirfenidone combination therapy. In non-experimental systems, a suitable control may be the tumor load present before administering the Type I interferon-receptor agonist and pirfenidone combination therapy. Other suitable controls may be a placebo control.

Whether growth of a tumor is inhibited can be determined using any known method, including, but not limited to, a proliferation assay as described in the Example; a ³H-thymidine uptake assay; and the like.

The methods are useful for treating a wide variety of cancers, including carcinomas, sarcomas, leukemias, and lymphomas.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

In many embodiments, the effective amounts of a Type I interferon receptor agonist and pirfenidone (or a pirfenidone analog) are synergistic amounts. As used herein, a “synergistic combination” or a “synergistic amount” of a Type I interferon receptor agonist and pirfenidone or a pirfenidone analog is a combined dosage that is more effective in the therapeutic or prophylactic treatment of cancer than the incremental improvement in treatment outcome that could be predicted or expected from a merely additive combination of (i) the therapeutic or prophylactic benefit of a Type I interferon receptor agonist when administered at that same dosage as a monotherapy and (ii) the therapeutic or prophylactic benefit of pirfenidone or a pirfenidone analog when administered at the same dosage as a monotherapy.

In some embodiments of the invention, a selected amount of a Type I interferon receptor agonist and a selected amount of pirfenidone or a pirfenidone analog are effective when used in combination therapy for a disease, but the selected amount of Type I interferon receptor agonist and/or the selected amount of pirfenidone or a pirfenidone analog is ineffective when used in monotherapy for the disease. Thus, the invention encompasses (1) regimens in which a selected amount of pirfenidone or a pirfenidone analog enhances the therapeutic benefit of a selected amount of Type I interferon receptor agonist when used in combination therapy for a disease, where the selected amount of pirfenidone or a pirfenidone analog provides no therapeutic benefit when used in monotherapy for the disease (2) regimens in which a selected amount of Type I interferon receptor agonist enhances the therapeutic benefit of a selected amount of pirfenidone or a pirfenidone analog when used in combination therapy for a disease, where the selected amount of Type I interferon receptor agonist provides no therapeutic benefit when used in monotherapy for the disease and (3) regimens in which a selected amount of Type I interferon receptor agonist and a selected amount of pirfenidone or a pifenidone analog provide a therapeutic benefit when used in combination therapy for a disease, where each of the selected amounts of Type I interferon receptor agonist and pirfenidone or a pirfenidone analog, respectively, provides no therapeutic benefit when used in monotherapy for the disease. As used herein, a “synergistically effective amount” of Type I interferon receptor agonist and pirfenidone or a pirfenidone analog, and its grammatical equivalents, shall be understood to include any regimen encompassed by any of (1)-(3) above.

Type I Interferon Receptor Agonist and IP-10 Combination Therapy

In some embodiments, the methods generally involve administering a therapeutically effective amount of a Type I receptor agonist and a therapeutically effective amount of IP-10. In some embodiments, the Type I receptor agonist is an IFN-α.

The methods are effective to reduce a tumor load by at least about 5%, at least about 10%, at least about 20%, at least about 25%, at least about 50%, at least about 75%, at least about 85%, or at least about 90%, up to total eradication of the tumor, when compared to a suitable control. Thus, in these embodiments, “effective amounts” of a Type I receptor agonist and IP-10 are amounts of a Type I receptor agonist and IP-10 that are sufficient to reduce tumor load by at least about 5%, at least about 10%, at least about 20%, at least about 25%, at least about 50%, at least about 75%, at least about 85%, or at least about 90%, up to total eradication of the tumor, when compared to a suitable control. In an experimental animal system, a suitable control may be a genetically identical animal not treated with the Type I receptor agonist and IP-10 combination therapy. In non-experimental systems, a suitable control may be the tumor load present before administering the Type I receptor agonist and IP-10 combination therapy. Other suitable controls may be a placebo control.

Whether a tumor load has been decreased can be determined using any known method, including, but not limited to, measuring solid tumor mass; counting the number of tumor cells using cytological assays; fluorescence-activated cell sorting (e.g., using antibody specific for a tumor-associated antigen) to determine the number of cells bearing a given tumor antigen; computed tomography scanning, magnetic resonance imaging, and/or x-ray imaging of the tumor to estimate and/or monitor tumor size; measuring the amount of tumor-associated antigen in a biological sample, e.g., blood; and the like.

The methods are effective to reduce the growth rate of a tumor by at least about 5%, at least about 10%, at least about 20%, at least about 25%, at least about 50%, at least about 75%, at least about 85%, or at least about 90%, up to total inhibition of growth of the tumor, when compared to a suitable control. Thus, in these embodiments, “effective amounts” of a Type I receptor agonist and IP-10 are amounts of a Type I receptor agonist and IP-10 that are sufficient to reduce tumor growth rate by at least about 5%, at least about 10%, at least about 20%, at least about 25%, at least about 50%, at least about 75%, at least about 85%, or at least about 90%, up to total inhibition of tumor growth, when compared to a suitable control. In an experimental animal system, a suitable control may be a genetically identical animal not treated with the Type I receptor agonist and IP-10 combination therapy. In non-experimental systems, a suitable control may be the tumor load present before administering the Type I receptor agonist and IP-10 combination therapy. Other suitable controls may be a placebo control.

Whether growth of a tumor is inhibited can be determined using any known method, including, but not limited to, a proliferation assay as described in the Example; a ³H-thymidine uptake assay; and the like.

The methods are useful for treating a wide variety of cancers, including carcinomas, sarcomas, leukemias, and lymphomas.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

In many embodiments, the effective amounts of a Type I receptor agonist and IP-10 are synergistic amounts. As used herein, a “synergistic combination” or a “synergistic amount” of a Type I receptor agonist and IP-10 is a combined dosage that is more effective in the therapeutic or prophylactic treatment of cancer than the incremental improvement in treatment outcome that could be predicted or expected from a merely additive combination of (i) the therapeutic or prophylactic benefit of a Type I receptor agonist when administered at that same dosage as a monotherapy and (ii) the therapeutic or prophylactic benefit of IP-10 when administered at the same dosage as a monotherapy.

In some embodiments of the invention, a selected amount of a Type I receptor agonist and a selected amount of IP-10 are effective when used in combination therapy for a disease, but the selected amount of Type I receptor agonist and/or the selected amount of IP-10 is ineffective when used in monotherapy for the disease. Thus, the invention encompasses (1) regimens in which a selected amount of IP-10 enhances the therapeutic benefit of a selected amount of Type I receptor agonist when used in combination therapy for a disease, where the selected amount of IP-10 provides no therapeutic benefit when used in monotherapy for the disease (2) regimens in which a selected amount of Type I receptor agonist enhances the therapeutic benefit of a selected amount of IP-10 when used in combination therapy for a disease, where the selected amount of Type I receptor agonist provides no therapeutic benefit when used in monotherapy for the disease and (3) regimens in which a selected amount of Type I receptor agonist and a selected amount of IP-10 provide a therapeutic benefit when used in combination therapy for a disease, where each of the selected amounts of Type I receptor agonist and IP-10, respectively, provides no therapeutic benefit when used in monotherapy for the disease. As used herein, a “synergistically effective amount” of Type I receptor agonist and IP-10, and its grammatical equivalents, shall be understood to include any regimen encompassed by any of (1)-(3) above.

Pirfenidone or a Pirfenidone Analog in Combination with Cancer Therapy

The present invention provides methods of treating cancer in an individual in need thereof, e.g., an individual having a cancer. The methods generally involve administering a therapeutically effective amount of pirfenidone, in combination with any other therapy for cancer.

The methods are effective to reduce a tumor load or reduce tumor progression by at least about 5%, at least about 10%, at least about 20%, at least about 25%, at least about 50%, at least about 75%, at least about 85%, or at least about 90%, up to total eradication of the tumor or inhibition of tumor progression, when compared to a suitable control. Thus, in these embodiments, “effective amounts” of pirfenidone are amounts of pirfenidone that in combination with another therapy for cancer are sufficient to reduce tumor load or reduce tumor progression by at least about 5%, at least about 10%, at least about 20%, at least about 25%, at least about 50%, at least about 75%, at least about 85%, or at least about 90%, up to total eradication of the tumor, or total inhibition of tumor progression, when compared to a suitable control. In an experimental animal system, a suitable control may be a genetically identical animal not treated with the pirfenidone combination therapy. In non-experimental systems, a suitable control may be the tumor load present before administering the pirfenidone combination therapy. Other suitable controls may be a placebo control.

Whether a tumor load has been decreased can be determined using any known method, including, but not limited to, measuring solid tumor mass; counting the number of tumor cells using cytological assays; fluorescence-activated cell sorting (e.g., using antibody specific for a tumor-associated antigen) to determine the number of cells bearing a given tumor antigen; computed tomography scanning, magnetic resonance imaging, and/or x-ray imaging of the tumor to estimate and/or monitor tumor size; measuring the amount of tumor-associated antigen in a biological sample, e.g., blood; and the like.

The methods are effective to reduce the growth rate of a tumor by at least about 5%, at least about 10%, at least about 20%, at least about 25%, at least about 50%, at least about 75%, at least about 85%, or at least about 90%, up to total inhibition of growth of the tumor, when compared to a suitable control. Thus, in these embodiments, “effective amounts” of pirfenidone and an additional agent are amounts of pirfenidone and the additional agent that are sufficient to reduce tumor growth rate by at least about 5%, at least about 10%, at least about 20%, at least about 25%, at least about 50%, at least about 75%, at least about 85%, or at least about 90%, up to total inhibition of tumor growth, when compared to a suitable control. In an experimental animal system, a suitable control may be a genetically identical aninmal not treated with the pirfenidone and the additional agent. In non-experimental systems, a suitable control may be the tumor load present before administering the pirfenidone and the additional agent. Other suitable controls may be a placebo control.

Whether growth of a tumor is inhibited can be determined using any known method, including, but not limited to, a proliferation assay as described in the Example; a ³H-thymidine uptake assay; and the like.

The methods are useful for treating a wide variety of cancers, including carcinomas, sarcomas, leukemias, and lymphomas.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

In many embodiments, the effective amounts of pirfenidone (or a pirfenidone analog) and an additional antineoplastic agent or biological response modifier are synergistic amounts. As used herein, a “synergistic combination” or a “synergistic amount” of pirfenidone or a pirfenidone analog and an additional antineoplastic/biological response modifier is a combined dosage that is more effective in the therapeutic or prophylactic treatment of cancer than the incremental improvement in treatment outcome that could be predicted or expected from a merely additive combination of (i) the therapeutic or prophylactic benefit of pirfenidone or a pirfenidone analog when administered at the same dosage as a monotherapy and (ii) the therapeutic or prophylactic benefit of the additional antineoplastic/biological response modifier when administered at that same dosage as a monotherapy.

In some embodiments of the invention, a selected amount of pirfenidone or a pirfenidone analog and a selected amount of an additional antineoplastic agent or biological response modifier are effective when used in combination therapy for a disease, but the selected amount of pirfenidone or a pirfenidone analog and/or the selected amount of the additional antineoplastic agent or biological response modifier is ineffective when used in monotherapy for the disease. Thus, the invention encompasses (1) regimens in which a selected amount of pirfenidone or a pirfenidone analog enhances the therapeutic benefit of a selected amount of an additional antineoplastic agent or biological response modifier when used in combination therapy for a disease, where the selected amount of pirfenidone or a pirfenidone analog provides no therapeutic benefit when used in monotherapy for the disease (2) regimens in which a selected amount of an additional antineoplastic agent or biological response modifier enhances the therapeutic benefit of a selected amount of pirfenidone or a pirfenidone analog when used in combination therapy for a disease, where the selected amount of the additional antineoplastic agent or biological response modifier provides no therapeutic benefit when used in monotherapy for the disease and (3) regimens in which a selected amount of an additional antineoplastic agent or biological response modifier and a selected amount of pirfenidone or a pifenidone analog provide a therapeutic benefit when used in combination therapy for a disease, where each of the selected amount of the additional antineoplastic agent or biological response modifier and the selected amount of pirfenidone or a pirfenidone analog, respectively, provides no therapeutic benefit when used in monotherapy for the disease. As used herein, a “synergistically effective amount” of pirfenidone or a pirfenidone analog and an additional agent, and its grammatical equivalents, shall be understood to include any regimen encompassed by any of (1)-(3) above.

IP-10

The term “IP-10” as used herein, refers to biologically active polypeptides having the amino acid sequence of the 77-amino acid mature, secreted form of human IP-10, referred to herein as “IP-10₂₂₋₉₈” (Luster et al. (1985) Nature 315: 672-676); active fragments of IP-10₂₂₋₉₈; fusion proteins comprising IP-10; variants of IP-10 comprising one or more amino acid differences from a naturally-occurring IP-10 polypeptide; and derivatives of IP-10, including, e.g., derivatives with N-terminal modifications.

Variants and derivatives of IP-10 that are suitable for use in a subject method are biologically active, e.g., such variants and derivatives typically retain at least about 40%, at least about 50%, at least about 60%, at least about 70%, at least about 80%, or at least about 90%, or more, of the biological activity of a naturally-occurring IP-10 polypeptide.

The amino acid sequence of human IP-10 is found under GenBank Accession No. NP_{—}001556. In many embodiments, human IP-10 having amino acids 22-98 as set forth in GenBank NP_{—}001566 is used.

The term “IP-10” includes a 75-amino acid form of IP-10 that lacks the two amino-terminal amino acids found in the mature form of IP-10 (Proost et al. (2001) Blood 98:3554-3561). The term “IP-10” includes biologically active fragments, including those described in U.S. Pat. No. 5,994,292. The term “IP-10” includes IP-10 with N-terminal modifications, including those described in WO 99/20759.

The term “IP-10” includes recombinant human IP-10₂₂₋₉₈. Recombinant human IP-10 is available from a variety of sources, including, e.g., PeproTech, Cell Sciences, BD Biosciences Pharmingen, Chemicon, PerkinElmer, and Serologicals. The term “IP-10” further includes recombinant human IP-10 that may include an N-terminal methionine residue not found in native IP-10. For example, recombinant human IP-10 may include IP-10₂₂₋₉₈ that further includes an additional N-terminal methionine added by a bacterium during synthesis. In some embodiments, recombinant IP-10 contains a mixture of IP-10₂₂₋₉₈ containing N-terminal methionine and IP-10₂₂₋₉₈ lacking N-terminal methionine.

The amino acid sequence of the IP-10 polypeptide may be altered in various ways known in the art to generate targeted changes in sequence. A variant polypeptide will usually be substantially similar to an IP-10 amino acid sequence discussed above, e.g., will differ by at least one amino acid, and may differ by at least two but typically not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (Oysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included for use in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using high performance liquid chromatography, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Pirfenidone and Analogs Thereof

Pirfenidone (5-methyl-1-phenyl-2-(1H)-pyridone) and specific pirfenidone analogs are disclosed for the treatment of cancer.

### Pirfenidone

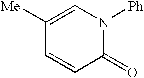

### Pirfenidone Analogs

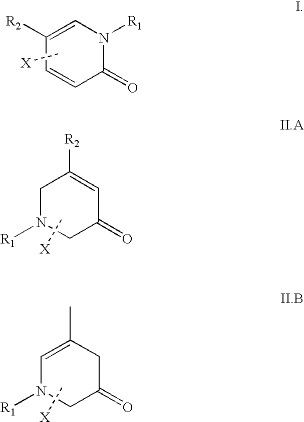 Descriptions for Substituents R₁, R₂, X

R₁: carbocyclic (saturated and unsaturated), heterocyclic (saturated or unsaturated), alkyls (saturated and unsaturated). Examples include phenyl, benzyl, pyrimidyl, naphthyl, indolyl, pyrrolyl, furyl, thienyl, imidazolyl, cyclohexyl, piperidyl, pyrrolidyl, morpholinyl, cyclohexenyl, butadienyl, and the like.

R₁ can further include substitutions on the carbocyclic or heterocyclic moieties with substituents such as halogen, nitro, amino, hydroxyl, alkoxy, carboxyl,-cyano, thio, alkyl, aryl, heteroalkyl, heteroaryl and combinations thereof, for example, 4-nitrophenyl, 3-chlorophenyl, 2,5-dinitrophenyl, 4-methoxyphenyl, 5-methyl-pyrrolyl, 2,5-dichlorocyclohexyl, guanidinyl-cyclohexenyl and the like.

R₂: alkyl, carbocylic, aryl, heterocyclic. Examples include: methyl, ethyl, propyl, isopropyl, phenyl, 4-nitrophenyl, thienyl and the like.

X: may be any number (from 1 to 3) of substituents on the carbocyclic or heterocyclic ring. The substituents can be the same or different. Substituents can include hydrogen, alkyl, heteroalkyl, aryl, heteroaryl, halo, nitro, carboxyl, hydroxyl, cyano, amino, thio, alkylamino, haloaryl and the like.

The substituents may be optionally further substituted with 1-3 substituents from the group consisting of alkyl, aryl, nitro, alkoxy, hydroxyl and halo groups. Examples include: methyl, 2,3-dimethyl, phenyl, p-tolyl, 4-chlorophenyl, 4-nitrophenyl, 2,5-dichlorophenyl, furyl, thienyl and the like.

Specific Examples include those shown in Table 1:

TABLE 1

| IA | IIB |
|---|---|
| 5-Methyl-1-(2′-pyridyl)-2-(1H) pyridine, | 6-Methyl-1-phenyl-3-(1H) pyridone, |
| 6-Methyl-1-phenyl-2-(1H) pyridone, | 5-Methyl-1-p-tolyl-3-(1H) pyridone, |
| 5-Methyl-3-phenyl-1-(2′-thienyl)-2-(1H) | 5-Methyl-1-(2′-naphthyl)-3-(1H) pyridone, |
| pyridone, | |
| 5-Methyl-1-(2′-naphthyl)-2-(1H) pyridone, | 5-Methyl-1-phenyl-3-(1H) pyridone, |
| 5-Methyl-1-p-tolyl-2-(1H) pyridone, | 5-Methyl-1-(5′-quinolyl)-3-(1H) pyridone, |
| 5-Methyl-1-(1′naphthyl)-2-(1H) pyridone, | 5-Ethyl-1-phenyl-3-(1H) pyridone, |
| 5-Ethyl-1-phenyl-2-(1H) pyridone, | 5-Methyl-1-(4′-methoxyphenyl)-3-(1H) pyridone, |
| 5-Methyl-1-(5′-quinolyl)-2-(1H) pyridone, | 4-Methyl-1-phenyl-3-(1H) pyridone, |
| 5-Methyl-1-(4′-quinolyl)-2-(1H) pyridone, | 5-Methyl-1-(3′-pyridyl)-3-(1H) pyridone, |
| 5-Methyl-1-(4′-pyridyl)-2-(1H) pyridone, | 5-Methyl-1-(2′-Thienyl)-3-(1H) pyridone, |
| 3-Methyl-1-phenyl-2-(1H) pyridone, | 5-Methyl-1-(2′-pyridyl)-3-(1H) pyridone, |
| 5-Methyl-1-(4′-methoxyphenyl)-2-(1H) | 5-Methyl-1-(2′-quinolyl)-3-(1H) pyridone, |
| pyridone, | |
| 1-Phenyl-2-(1H) pyridone, | 1-Phenyl-3-(1H) pyridine, |
| 1,3-Diphenyl-2-(1H) pyridone, | 1-(2′-Furyl)-5-methyl-3-(1H) pyridone, |
| 1,3-Diphenyl-5-methyl-2-(1H) pyridone, | 1-(4′-Chlorophenyl)-5-methyl-3-(1H) pyridine. |
| 5-Methyl-1-(3′-trifluoromethylphenyl)-2- | |
| (1H)-pyridone, | |
| 3-Ethyl-1-phenyl-2-(1H) pyridone, | |
| 5-Methyl-1-(3′-pyridyl)-2-(1H) pyridone, | |
| 5-Methyl-1-(3-nitrophenyl)-2-(1H) | |
| pyridone, | |
| 3-(4′-Chlorophenyl)-5-Methyl-1-phenyl-2- | |
| (1H) pyridone, | |
| 5-Methyl-1-(2′-Thienyl)-2-(1H) pyridone, | |
| 5-Methyl-1-(2′-thiazolyl)-2-(1H) pyridone, | |
| 3,6-Dimethyl-1-phenyl-2-(1H) pyridone, | |
| 1-(4′Chlorophenyl)-5-Methyl-2-(1H) | |
| pyridone, | |
| 1-(2′-Imidazolyl)-5-Methyl-2-(1H) | |
| pyridone, | |
| 1-(4′-Nitrophenyl)-2-(1H) pyridone, | |
| 1-(2′-Furyl)-5-Methyl-2-(1H) pyridone, | |
| 1-Phenyl-3-(4′-chlorophenyl)-2-(1H) | |
| pyridine. | |

U.S. Pat. Nos. 3,974,281; 3,839,346; 4,042,699; 4,052,509; 5,310,562; 5,518,729; 5,716,632; and 6,090,822 describe methods for the synthesis and formulation of pirfenidone and specific pirfenidone analogs in pharmaceutical compositions suitable for use in the methods of the present invention.

Type I Interferon Receptor Agonists

Type I interferon receptor agonists suitable for use in a subject method include an IFN-α; an IFN-β; an IFN-tau; an IFN-ω; antibody agonists specific for a Type I interferon receptor; and any other agonist of Type I interferon receptor, including non-polypeptide agonists.

IFN-α

The term “interferon-alpha” as used herein refers to a family of related polypeptides that inhibit viral replication and cellular proliferation and modulate immune response. The term “IFN-α” includes IFN-α polypeptides that are naturally occurring; non-naturally-occurring IFN-α polypeptides; and analogs of naturally occurring or non-naturally occurring IFN-α that retain antiviral activity of a parent naturally-occurring or non-naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron®A interferon available from Schering Corporation, Kenilworth, N.J.; recombinant interferon alpha-2a such as Roferon® interferon available from Hoffmann-La Roche, Nutley, N.J.; recombinant interferon alpha-2C such as Berofor® alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon® interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon® Tradename.

The term “IFN-α,” as used herein, also encompasses consensus IFN-α. As used herein, the term “consensus IFN-α” refers to a non-naturally-occurring polypeptide, which includes those amino acid residues that are common to all naturally-occurring human leukocyte IFN-α subtype sequences and which includes, at one or more of those positions where there is no amino acid common to all subtypes, an amino acid which predominantly occurs at that position, provided that at any such position where there is no amino acid common to all subtypes, the polypeptide excludes any amino acid residue which is not present in at least one naturally-occurring subtype. Amino acid residues that are common to all naturally-occurring human leukocyte IFN-α subtype sequences (“common amino acid residues”), and amino acid residues that occur predominantly at non-common residues (“consensus amino acid residues”) are known in the art.

Consensus IFN-α (also referred to as “CIFN” and “IFN-con” and “consensus interferon”) encompasses but is not limited to the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con₁ is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods. Use of CIFN is of particular interest.

Also suitable for use in the present invention are fusion polypeptides comprising an IFN-α and a heterologous polypeptide. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in the present invention are gene-shuffled forms of IFN-α. See., e.g., Masci et al. (2003) Curr. Oncol. Rep. 5:108-113.

IFN-α polypeptides can be produced by any known method. DNA sequences encoding IFN-con may be synthesized as described in the above-mentioned patents or other standard methods. In many embodiments, IFN-α polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-α is “recombinant IFN-α.” Where the host cell is a bacterial host cell, the IFN-α is modified to comprise an N-terminal methionine. IFN-α produced in E. coli is generally purified by procedures known to those skilled in the art and generally described in Klein et al. ((1988) J. Chromatog. 454:205-215) for IFN-con₁.

Bacterially produced IFN-α may comprise a mixture of isoforms with respect to the N-terminal amino acid residue. For example, purified IFN-con may comprise a mixture of isoforms with respect to the N-terminal methionine status. For example, in some embodiments, an IFN-con comprises a mixture of N-terminal methionyl IFN-con, des-methionyl IFN-con with an unblocked N-terminus, and des-methionyl IFN-con with a blocked N-terminus. As one non-limiting example, purified IFN-con₁ comprises a mixture of methionyl IFN-con₁ des-methionyl IFN-con₁ and des-methionyl IFN-con₁ with a blocked N-terminus. Klein et al. ((1990) Arch. Biochemistry & Biophys. 276:531-537). Alternatively, IFN-con may comprise a specific, isolated isoform. Isoforms of IFN-con are separated from each other by techniques such as isoelectric focusing which are known to those skilled in the art.

It is to be understood that IFN-α as described herein may comprise one or more modified amino acid residues, e.g., glycosylations, chemical modifications, and the like.

PEGylated IFN-α

The term “IFN-α” also encompasses derivatives of IFN-α that are derivatized (e.g., are chemically modified) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffmann La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

Any of the above-mentioned IFN-α polypeptides can be modified with one or more polyethylene glycol moieties, i.e., PEGylated. The PEG molecule of a PEGylated IFN-α polypeptide is conjugated to one or more amino acid side chains of the IFN-α polypeptide. In some embodiments, the PEGylated IFN-α contains a PEG moiety on only one amino acid. In other embodiments, the PEGylated IFN-α contains a PEG moiety on two or more amino acids, e.g., the IFN-α contains a PEG moiety attached to two, three, four, five, six, seven, eight, nine, or ten different amino acid residues.

IFN-α may be coupled directly to PEG (i.e., without a linking group) through an amino group, a sulfhydryl group, a hydroxyl group, or a carboxyl group. In some embodiments, the PEGylated IFN-α is PEGylated at or near the amino terminus (N-terminus) of the IFN-α polypeptide, e.g., the PEG moiety is conjugated to the IFN-α polypeptide at one or more amino acid residues from amino acid 1 through amino acid 4, or from amino acid 5 through about 10.

In other embodiments, the PEGylated IFN-α is PEGylated at one or more amino acid residues from about 10 to about 28.

In other embodiments, the PEGylated IFN-α is PEGylated at or near the carboxyl terminus (C-terminus) of the IFN-α polypeptide, e.g., at one or more residues from amino acids 156-166, or from amino acids 150 to 155.

In other embodiments, the PEGylated IFN-α is PEGylated at one or more amino acid residues at one or more residues from amino acids 100-114.

Selection of the attachment site of polyethylene glycol on the IFN-α is determined by the role of each of the sites within the receptor-binding and/or active site domains of the protein, as would be known to the skilled artisan. In general, amino acids at which PEGylation is to be avoided include amino acid residues from amino acid 30 or amino acid 40; and amino acid residues from amino acid 113 to amino acid 149.

In some embodiments, PEG is attached to IFN-α via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. Methods for making succinimidyl propionate (SPA) and succinimidyl butanoate (SBA) ester-activated PEGs are described in U.S. Pat. No. 5,672,662 (Harris, et al.) and WO 97/03106.

Methods for attaching a PEG to an IFN-α polypeptide are known in the art, and any known method can be used. See, for example, by Park et al, Anticancer Res., 1:373-376 (1981); Zaplipsky and Lee, Polyethylene Glycol Chemistry: Biotechnical and Biomedical Applications, J. M. Harris, ed., Plenum Press, NY, Chapter 21 (1992); and U.S. Pat. No. 5,985,265.

Pegylated IFN-α, and methods for making same, are discussed in, e.g., U.S. Pat. Nos. 5,382,657; 5,981,709; 5,985,265; and 5,951,974. Pegylated IFN-α encompasses conjugates of PEG and any of the above-described IFN-α molecules, including, but not limited to, PEG conjugated to interferon alpha-2a (Roferon, Hoffman LaRoche, Nutley, N.J.), where PEGylated Roferon is known as PEGASYS® (Hoffman LaRoche); interferon alpha 2b (Intron, Schering-Plough, Madison, N.J.), where PEGylated Intron is known as PEG-INTRON® (Schering-Plough); interferon alpha-2c (Berofor Alpha, Boehringer Ingelheim, Ingelheim, Germany); and consensus interferon (CIFN) as defined by determination of a consensus sequence of naturally occurring interferon alphas (Infergen, Amgen, Thousand Oaks, Calif.), where PEGylated Infergen is referred to as PEG-INFERGEN®.

Generally, the PEG moiety is linked to a surface-exposed lysine (“lys”) residue. Whether a lysine is surface exposed can be determined using any known method. Generally, analysis of hydrophilicity (e.g., Kyte-Doolittle and Hoppe-Woods analysis) and/or predicted surface-forming regions (e.g., Emini surface-forming probability analysis) is carried out using appropriate computer programs, which are well known to those skilled in the art. Suitable computer programs include PeptideStructure, and the like. Alternatively, NMR investigations can identify the surface accessible residues by virtue of the chemical shift of the protons of a specific functional group in the spectrum. In other cases, the inaccessibility or accessibility of residues to solvents or environment can be assessed by fluorescence. In yet other cases, the surface exposure of accessible lysines can be ascertained by the chemical reactivity to water soluble reagents e.g., Trinitrobenzene sulfonate or TNBS, and like measurements.

In many embodiments, the PEG is a monomethoxyPEG molecule that reacts with primary amine groups on the IFN-α polypeptide. Methods of modifying polypeptides with monomethoxy PEG via reductive alkylation are known in the art. See, e.g., Chamow et al. (1994) Bioconj. Chem. 5:133-140.

In one non-limiting example, PEG is linked to IFN-α via an SPA linking group. SPA esters of PEG, and methods for making same, are described in U.S. Pat. No. 5,672,662. SPA linkages provide for linkage to free amine groups on the IFN-α polypeptide.

For example, a PEG molecule is covalently attached via a linkage that comprises an amide bond between a propionyl group of the PEG moiety and the epsilon amino group of a surface-exposed lysine residue in the IFN-α polypeptide. Such a bond can be formed, e.g., by condensation of an a-methoxy, omega propanoic acid activated ester of PEG (mPEGspa).

In some embodiments, the PEGylated IFN-α is a monoPEGylated IFN-α. In other embodiments, the monoPEGylated IFN-α is an IFN-α polypeptide covalently linked to a single PEG moiety via a lysine residue or the N-terminal amino acid residue of the IFN-α polypeptide. In other embodiments, the monoPEGylated IFN-α is an IFN-α polypeptide covalently linked to a single PEG moiety via an amide bond between either the epsilon-amino group of a lysine residue or the alpha-amino group of the IFN-α polypeptide and an activated carboxyl group of the PEG moiety. In other embodiments, the monoPEGylated IFN-α is an IFN-α polypeptide covalently linked to a single, linear PEG moiety. In other embodiments, the monoPEGylated IFN-α is an IFN-α polypeptide covalently linked to a single, linear 30 kD PEG moiety. In other embodiments, the monoPEGylated IFN-α is an IFN-α polypeptide covalently linked to a single, linear 30 kD PEG moiety via an amide bond between the epsilon-amino group of a lysine residue or the alpha-amino group of the IFN-α polypeptide and an activated carboxyl group of the PEG moiety. In other embodiments, the monoPEGylated IFN-α is an IFN-α polypeptide covalently linked to a single, linear 30 kD PEG via an amide bond between the epsilon-amino group of a lysine residue or the alpha-amino group of the IFN-α polypeptide and an activated propionyl group of the PEG moiety. In other embodiments, the monoPEGylated IFN-α is an IFN-α polypeptide covalently linked to a single, linear monomethoxy-PEG (mPEG). In other embodiments, the monoPEGylated IFN-α is the product of a condensation reaction between an IFN-α polypeptide and a linear, succinimidyl propionate ester-activated 30 kD mPEG. In any of the foregoing methods using a PEGylated IFN-α, the IFN-α polypeptide can be a consensus interferon (CIFN) polypeptide. In any of the foregoing methods using a PEGylated IFN-α, the IFN-α polypeptide can be a CIFN polypeptide that is interferon alfacon-1.

In some embodiments, the PEGylated IFN-α comprises CIFN PEGylated at the epsilon amino group of a lysine residue.

As one non-limiting example, one monopegylated CIFN conjugate preferred for use herein has a linear PEG moiety of about 30 kD attached via a covalent linkage to the CIFN polypeptide, where the covalent linkage is an amide bond between a propionyl group of the PEG moiety and the epsilon amino group of a surface-exposed lysine residue in the CIFN polypeptide, where the surface-exposed lysine residue is chosen from lys³¹, lys⁵⁰, lys⁷¹, lys⁸⁴, lys¹²¹, lys¹²², lys¹³⁴, lys¹³⁵, and lys¹⁶⁵, and the amide bond is formed by condensation of an α-methoxy, omega propanoic acid activated ester of PEG.

Polyethylene Glycol

Polyethylene glycol suitable for conjugation to an IFN-α polypeptide is soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

In many embodiments, PEG has at least one hydroxyl group, e.g., a terminal hydroxyl group, which hydroxyl group is modified to generate a functional group that is reactive with an amino group, e.g., an epsilon amino group of a lysine residue, a free amino group at the N-terminus of a polypeptide, or any other amino group such as an amino group of asparagine, glutamine, arginine, or histidine.

In other embodiments, PEG is derivatized so that it is reactive with free carboxyl groups in the IFN-α polypeptide, e.g., the free carboxyl group at the carboxyl terminus of the IFN-α polypeptide. Suitable derivatives of PEG that are reactive with the free carboxyl group at the carboxyl-terminus of IFN-α include, but are not limited to PEG-amine, and hydrazine derivatives of PEG (e.g., PEG-NH—NH₂).

In other embodiments, PEG is derivatized such that it comprises a terminal thiocarboxylic acid group, —COSH, which selectively reacts with amino groups to generate amide derivatives. Because of the reactive nature of the thio acid, selectivity of certain amino groups over others is achieved. For example, —SH exhibits sufficient leaving group ability in reaction with N-terminal amino group at appropriate pH conditions such that the ε-amino groups in lysine residues are protonated and remain non-nucleophilic. On the other hand, reactions under suitable pH conditions may make some of the accessible lysine residues to react with selectivity.

In other embodiments, the PEG comprises a reactive ester such as an N-hydroxy succinimidate at the end of the PEG chain. Such an N-hydroxysuccinimidate-containing PEG molecule reacts with select amino groups at particular pH conditions such as neutral 6.5-7.5. For example, the N-terminal amino groups may be selectively modified under neutral pH conditions. However, if the reactivity of the reagent were extreme, accessible-NH₂ groups of lysine may also react.

The PEG can be conjugated directly to the IFN-α polypeptide, or through a linker. In some embodiments, a linker is added to the IFN-α polypeptide, forming a linker-modified IFN-α polypeptide. Such linkers provide various functionalities, e.g., reactive groups such sulfhydryl, amino, or carboxyl groups to couple a PEG reagent to the linker-modified IFN-α polypeptide.

In some embodiments, the PEG conjugated to the IFN-α polypeptide is linear. In other embodiments, the PEG conjugated to the IFN-α polypeptide is branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

PEG having a molecular weight in a range of from about 2 kDa to about 100 kDa, is generally used, where the term “about,” in the context of PEG, indicates that in preparations of polyethylene glycol, some molecules will weigh more, some less, than the stated molecular weight. For example, PEG suitable for conjugation to IFN-α has a molecular weight of from about 2 kDa to about 5 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 40 kDa, from about 40 kDa to about 50 kDa, from about 50 kDa to about 60 kDa, from about 60 kDa to about 70 kDa, from about 70 kDa to about 80 kDa, from about 80 kDa to about 90 kDa, or from about 90 kDa to about 100 kDa.

Preparing PEG-IFN-α Conjugates

As discussed above, the PEG moiety can be attached, directly or via a linker, to an amino acid residue at or near the N-terminus, internally, or at or near the C-terminus of the IFN-α polypeptide. Conjugation can be carried out in solution or in the solid phase.

N-Terminal Linkage

Methods for attaching a PEG moiety to an amino acid residue at or near the N-terminus of an IFN-α polypeptide are known in the art. See, e.g., U.S. Pat. No. 5,985,265.

In some embodiments, known methods for selectively obtaining an N-terminally chemically modified IFN-α are used. For example, a method of protein modification by reductive alkylation which exploits differential reactivity of different types of primary amino groups (lysine versus the N-terminus) available for derivatization in a particular protein can be used. Under the appropriate reaction conditions, substantially selective derivatization of the protein at the N-terminus with a carbonyl group containing polymer is achieved. The reaction is performed at pH which allows one to take advantage of the pKₐ differences between the ε-amino groups of the lysine residues and that of the a-amino group of the N-terminal residue of the protein. By such selective derivatization attachment of a PEG moiety to the IFN-α is controlled: the conjugation with the polymer takes place predominantly at the N-terminus of the IFN-α and no significant modification of other reactive groups, such as the lysine side chain amino groups, occurs.

C-Terminal Linkage

N-terminal-specific coupling procedures such as described in U.S. Pat. No. 5,985,265 provide predominantly monoPEGylated products. However, the purification procedures aimed at removing the excess reagents and minor multiply PEGylated products remove the N-terminal blocked polypeptides. In terms of therapy, such processes lead to significant increases in manufacturing costs. For example, examination of the structure of the well-characterized Infergen® Alfacon-1 CIFN polypeptide amino acid sequence reveals that the clipping is approximate 5% at the carboxyl terminus and thus there is only one major C-terminal sequence. Thus, in some embodiments, N-terminally PEGylated IFN-α is not used; instead, the IFN-α polypeptide is C-terminally PEGylated.

An effective synthetic as well as therapeutic approach to obtain mono PEGylated Infergen product is therefore envisioned as follows:

A PEG reagent that is selective for the C-terminal can be prepared with or without spacers. For example, polyethylene glycol modified as methyl ether at one end and having an amino function at the other end may be used as the starting material.

Preparing or obtaining a water-soluble carbodiimide as the condensing agent can be carried out. Coupling IFN-α (e.g., Infergen® Alfacon-1 CIFN or consensus interferon) with a water-soluble carbodiimide as the condensing reagent is generally carried out in aqueous medium with a suitable buffer system at an optimal pH to effect the amide linkage. A high molecular weight PEG can be added to the protein covalently to increase the molecular weight.

The reagents selected will depend on process optimization studies. A non-limiting example of a suitable reagent is EDAC or 1-ethyl-3-(3-dimethylaminopropyl)carbodiimide. The water solubility of EDAC allows for direct addition to a reaction without the need for prior organic solvent dissolution. Excess reagent and the isourea formed as the by-product of the cross-linking reaction are both water-soluble and may easily be removed by dialysis or gel filtration. A concentrated solution of EDAC in water is prepared to facilitate the addition of a small molar amount to the reaction. The stock solution is prepared and used immediately in view of the water labile nature of the reagent. Most of the synthetic protocols in literature suggest the optimal reaction medium to be in pH range between 4.7 and 6.0. However the condensation reactions do proceed without significant losses in yields up to pH 7.5. Water may be used as solvent. In view of the contemplated use of Infergen, preferably the medium will be 2-(N-morpholino)ethane sulfonic acid buffer pre-titrated to pH between 4.7 and 6.0. However, 0.1M phosphate in the pH 7-7.5 may also be used in view of the fact that the product is in the same buffer. The ratios of PEG amine to the IFN-α molecule is optimized such that the C-terminal carboxyl residue(s) are selectively PEGylated to yield monoPEGylated derivative(s).

Even though the use of PEG amine has been mentioned above by name or structure, such derivatives are meant to be exemplary only, and other groups such as hydrazine derivatives as in PEG-NH—NH₂ which will also condense with the carboxyl group of the IFN-α protein, can also be used. In addition to aqueous phase, the reactions can also be conducted on solid phase. Polyethylene glycol can be selected from list of compounds of molecular weight ranging from 300-40000. The choice of the various polyethylene glycols will also be dictated by the coupling efficiency and the biological performance of the purified derivative in vitro and in vivo i.e., circulation times, anti viral activities etc.

Additionally, suitable spacers can be added to the C-terminal of the protein. The spacers may have reactive groups such as SH, NH₂ or COOH to couple with appropriate PEG reagent to provide the high molecular weight IFN-α derivatives. A combined solid/solution phase methodology can be devised for the preparation of C-terminal pegylated interferons. For example, the C-terminus of IFN-α is extended on a solid phase using a Gly-Gly-Cys-NH₂ spacer and then monopegylated in solution using activated dithiopyridyl-PEG reagent of appropriate molecular weights. Since the coupling at the C-terminus is independent of the blocking at the N-terminus, the envisioned processes and products will be beneficial with respect to cost (a third of the protein is not wasted as in N-terminal PEGylation methods) and contribute to the economy of the therapy to treat chronic hepatitis C infections, liver fibrosis etc.

There may be a more reactive carboxyl group of amino acid residues elsewhere in the molecule to react with the PEG reagent and lead to monoPEGylation at that site or lead to multiple PEGylations in addition to the —COOH group at the C-terminus of the IFN-α. It is envisioned that these reactions will be minimal at best owing to the steric freedom at the C-terminal end of the molecule and the steric hindrance imposed by the carbodiimides and the PEG reagents such as in branched chain molecules. It is therefore the preferred mode of PEG modification for Infergen and similar such proteins, native or expressed in a host system, which may have blocked N-termini to varying degrees to improve efficiencies and maintain higher in vivo biological activity.

Another method of achieving C-terminal PEGylation is as follows. Selectivity of C-terminal PEGylation is achieved with a sterically hindered reagent which excludes reactions at carboxyl residues either buried in the helices or internally in IFN-α. For example, one such reagent could be a branched chain PEG ˜40 kd in molecular weight and this agent could be synthesized as follows:

OH₃C—(CH₂CH₂O)ₙ—CH₂CH₂NH₂+Glutamic Acid i.e., HOCO—CH₂CH₂CH(NH2)—COOH is condensed with a suitable agent e.g.,.dicyclohexyl carbodiimide or water-soluble EDC to provide the branched chain PEG agent OH₃C—(CH₂CH₂O)ₙ—CH₂CH₂NHCOCH(NH₂)CH₂OCH₃—(CH₂CH₂O)ₙ—CH₂CH₂NHCOCH₂.

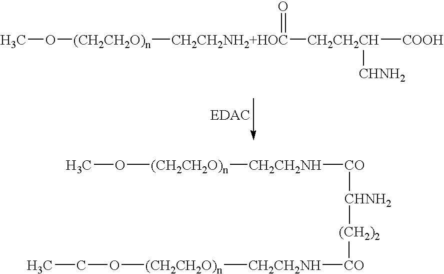

This reagent can be used in excess to couple the amino group with the free and flexible carboxyl group of IFN-α to form the peptide bond.

If desired, PEGylated IFN-α is separated from unPEGylated IFN-α using any known method, including, but not limited to, ion exchange chromatography, size exclusion chromatography, and combinations thereof. For example, where the PEG-IFN-α conjugate is a monoPEGylated IFN-α, the products are first separated by ion exchange chromatography to obtain material having a charge characteristic of monoPEGylated material (other multi-PEGylated material having the same apparent charge may be present), and then the monoPEGylated materials are separated using size exclusion chromatography.

Mixed Populations of IFN-α

In some embodiments, the IFN-α administered is a population of IFN-α polypeptides comprising PEGylated IFN-α polypeptides and non-PEGylated IFN-α polypeptides. Generally, a PEGylated IFN-α species represents from about 0.5% to about 99.5% of the total population of IFNα polypeptide molecules in a population, e.g, a given PEGylated IFN-α species represents about 0.5%, about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 99.5% of the total population of IFN-α polypeptide molecules in a population.

Type II Interferon Teceptor Agonists

Type II interferon receptor agonists suitable for use in a subject method include any naturally-occurring or non-naturally-occurring ligand of a human Type II interferon receptor which binds to and causes signal transduction via the receptor. Type II interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

A specific example of a Type II interferon receptor agonist is IFN-γ and variants thereof. While the present invention exemplifies use of an IFN-γ polypeptide, it will be readily apparent that any Type II interferon receptor agonist can be used in a subject method.

Interferon-Gamma

The nucleic acid sequences encoding IFN-γ polypeptides may be accessed from public databases, e.g., Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J.B.C. 259:6790.

IFN-γ1b (Actimmune®; human interferon) is a single-chain polypeptide of 140 amino acids. It is made recombinantly in E. coli and is unglycosylated. Rinderknecht et al. (1984) J. Biol. Chem. 259:6790-6797. Recombinant IFN-γ as discussed in U.S. Pat. No. 6,497,871 is also suitable for use herein.

The IFN-γ to be used in the methods of the present invention may be any of natural IFN-γs, recombinant IFN-γs and the derivatives thereof so far as they have an IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra, consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG that, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxyl terminus relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e., will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. In one embodiment, the invention contemplates the use of IFN-γ variants with one or more non-naturally occurring glycosylation and/or pegylation sites that are engineered to provide glycosyl- and/or PEG-derivatized polypeptides with reduced serum clearance, such as the IFN-γ polypeptide variants described in International Patent Publication No. WO 01/36001 or WO 02/081507. Also included are modifications of glycosylation, e.g., those made by modifying the glycosylation patterns of a polypeptide during its synthesis and processing or in further processing steps; e.g., by exposing the polypeptide to enzymes that affect glycosylation, such as mammalian glycosylating or deglycosylating enzymes. Also embraced are sequences that have phosphorylated amino acid residues, e.g., phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g., D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Dosages, Formulations, and Routes of Administration

In carrying out a subject method, an active agent (e.g., IP-10, pirfenidone or a pirfenidone analog, a Type II interferon receptor agonist, a Type I interferon receptor agonist, etc.) is administered to individuals in a formulation (e.g., in separate formulations) with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In the subject methods, the active agent(s) may be administered to the host using any convenient means capable of resulting in the desired therapeutic effect. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, intravenous, subcutaneous, intramuscular, intratumoral, transdermal, intratracheal, etc., administration. In some embodiments, two different routes of administration are used. For example, in some embodiments, IP-10 is administered by a route such as intramuscular, subcutaneous, or intravenous, and pirfenidone or pirfenidone analog is administered orally.

Subcutaneous administration of an active agent (e.g., IP-10, pirfenidone or a pirfenidone analog, a Type II interferon receptor agonist, a Type I interferon receptor agonist, etc.) is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of an active agent (e.g., IP-10, pirfenidone or a pirfenidone analog, a Type II interferon receptor agonist, a Type I interferon receptor agonist, etc.) to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, an active agent (e.g., IP-10, pirfenidone or a pirfenidone analog, a Type II interferon receptor agonist, a Type I interferon receptor agonist, etc.) is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, an active agent (e.g., IP-10, pirfenidone or a pirfenidone analog, a Type II interferon receptor agonist, a Type I interferon receptor agonist, etc.) is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are preferred in some embodiments because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are preferred in some embodiments due to their generally more consistent, controlled release over time. Osmotic pumps are particularly preferred in some embodiments due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent (e.g., IP-10, pirfenidone or a pirfenidone analog, a Type II interferon receptor agonist, a Type I interferon receptor agonist, etc.) is delivered using an implantable drug delivery system, e.g., a system that is programmable to provide for administration of the active agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of one or more active agents. Similarly, unit dosage forms for injection or intravenous administration may comprise the active agent(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent(s) calculated in an amount sufficient to produce the desired effect in association with a pharmaceutically acceptable diluent, carrier or vehicle. The specifications for the active agent(s) depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the administered agent is a polypeptide (e.g., an IP-10, or interferon receptor agonist, e.g., Type I, or Type II), a polynucleotide encoding the polypeptide may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells.

IP-10 and Pirfenidone or a Pirfenidone Analog in Combination Therapy

In some embodiments, pirfenidone or a pirfenidone analog is administered during the entire course of IP-10 treatment. In other embodiments, pirfenidone or a pirfenidone analog is administered for a period of time that is overlapping with that of the IP-10 treatment, e.g., the pirfenidone or pirfenidone analog treatment can begin before the IP-10 treatment begins and end before the IP-10 treatment ends; the pirfenidone or pirfenidone analog treatment can begin after the IP-10 treatment begins and end after the IP-10 treatment ends; the pirfenidone or pirfenidone analog treatment can begin after the IP-10 treatment begins and end before the IP-10 treatment ends; or the pirfenidone or pirfenidone analog treatment can begin before the IP-10 treatment begins and end after the IP-10 treatment ends.

Effective dosages of IP-10 range from 0.1 μg to 1000 ρg per dose, e.g., from about 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μ per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, or from about 900 μg to about a 1000 μg per dose.

In some embodiments, effective dosages of IP-10 are expressed as mg/kg body weight In these embodiments, effective dosages of IP-10 are from about 0.1 mg/kg body weight to about 10 mg/kg body weight, e.g., from about 0.1 mg/kg body weight to about 0.5 mg/kg body weight, from about 0.5 mg/kg body weight to about 1.0 mg/kg body weight, from about 1.0 mg/kg body weight to about 2.5 mg/kg body weight, from about 2.5 mg/kg body weight to about 5.0 mg/kg body weight, from about 5.0 mg/kg body weight to about 7.5 mg/kg body weight, or from about 7.5 mg/kg body weight to about 10 mg/kg body weight.

In many embodiments, IP-10 and/or pirfenidone or pirfenidone analog is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The IP-10 can be administered tid, bid, qd, qod, biw, tiw, qw, qow, three times per month, once monthly, substantially continuously, or continuously.

Those of skill will readily appreciate that dose levels can vary as a function of the specific agent used (e.g., the specific form of IP-10), the severity of the symptoms and the susceptibility of the subject to side effects. Preferred dosages for a given agent are readily determinable by those of skill in the art by a variety of means.

In many embodiments, multiple doses of IP-10 are administered. For example, IP-10 is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In general, effective dosages of pirfenidone or specific pirfenidone analogs can range from about 0.5 mg/kg/day to about 200 mg/kg/day, or at a fixed dosage of about 400 mg to about 3600 mg per day, or about 50 mg to about 10,000 mg per day, or about 100 mg to about 1,000 mg per day, or about 1,000 mg to about 3,000 mg per day, or about 1,000 mg to about 10,000 mg per day, administered orally, optionally in two or more divided doses per day. Other doses and formulations of pirfenidone and pirfenidone analogs suitable for use in a subject method for the treatment of cancer are described in U.S. Pat Nos. 3,974,281; 3,839,346; 4,042,699; 4,052,509; 5,310,562; 5,518,729; 5,716,632; and 6,090,822.

Those of skill in the art will readily appreciate that dose levels of pirfenidone or pirfenidone analog can vary as a function of the specific compound, the severity of the symptoms and the susceptibility of the subject to side effects. Preferred dosages for a given compound are readily determinable by those of skill in the art by a variety of means.

Pirfenidone (or a pirfenidone analog) can be administered daily, twice a day, or three times a day, or in divided daily doses ranging from 2 to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

IP-10 and pirfenidone (or pirfenidone analog) are generally administered in separate formulations. IP-10 and pirfenidone (or pirfenidone analog) may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

In one embodiment, the invention provides a method using an effective amount of IP-10 and pirfenidone or a specific pirfenidone analog in the treatment of cancer in a patient comprising administering to the patient a dosage of IP-10 containing an amount of from about 0.1 μg to about 1000 μg of drug per dose of IP-10, subcutaneously qd, qod, tiw, or biw, or per day substantially continuously or continuously, in combination with a dosage of pirfenidone or a specific pirfenidone analog containing an amount of about 100 mg to about 1,000 mg of drug per dose orally qd, optionally in two or more divided doses per day, for the desired treatment duration.

In one embodiment, the invention provides a method using an effective amount of IP-10 and pirfenidone or a specific pirfenidone analog in the treatment of cancer in a patient comprising administering to the patient a dosage of IP-10 containing an amount of from about 0.1 μg to about 1000 μg of drug per dose of IP-10, intramuscularly qd, qod, tiw, or biw, or per day substantially continuously or continuously, in combination with a dosage of pirfenidone or a specific pirfenidone analog containing an amount of about 100 mg to about 1,000 mg of drug per dose orally qd, optionally in two or more divided doses per day, for the desired treatment duration.

In one embodiment, the invention provides a method using an effective amount of IP-10 and pirfenidone or a specific pirfenidone analog in the treatment of cancer in a patient comprising administering to the patient a dosage of IP-10 containing an amount of from about 0.1 μg to about 1000 μg of drug per dose of IP-10, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, for the desired treatment duration.

In one embodiment, the invention provides a method using an effective amount of IP-10 and pirfenidone or a specific pirfenidone analog in the treatment of cancer in a patient comprising administering to the patient a dosage of IP-10 containing an amount of from about 0.1 μg to about 1000 μg of drug per dose of IP-10, intramuscularly qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, for the desired treatment duration.

In one embodiment, the invention provides a method using an effective amount of IP-10 and pirfenidone or a specific pirfenidone analog in the treatment of cancer in a patient comprising administering to the patient a dosage of IP-10 containing an amount of from about 0.1 μg to about 50 μg of drug per dose of IP-10, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, for the desired treatment duration.

In one embodiment, the invention provides a method using an effective amount of IP-10 and pirfenidone or a specific pirfenidone analog in the treatment of cancer in a patient comprising administering to the patient a dosage of IP-10 containing an amount of from about 0.1 μg to about 50 μg of drug per dose of IP-10, intramuscularly qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, for the desired treatment duration.

Synergistic Combinations of IP-10 and Pirfenidone

In many embodiments, the effective amounts of IP-10 and pirfenidone (or a pirfenidone analog) are synergistic amounts. As used herein, a “synergistic combination” or a “synergistic amount” of IP-10 and pirfenidone or a pirfenidone analog is a combined dosage that is more effective in the therapeutic or prophylactic treatment of cancer than the incremental improvement in treatment outcome that could be predicted or expected from a merely additive combination of (i) the therapeutic or prophylactic benefit of IP-10 when administered at that same dosage as a monotherapy and (ii) the therapeutic or prophylactic benefit of pirfenidone or a pirfenidone analog when administered at the same dosage as a monotherapy.

In some embodiments of the invention, a selected amount of IP-10 and a selected amount of pirfenidone or a pirfenidone analog are effective when used in combination therapy for a disease, but the selected amount of IP-10 and/or the selected amount of pirfenidone or a pirfenidone analog is ineffective when used in monotherapy for the disease. Thus, the invention encompasses (1) regimens in which a selected amount of pirfenidone or a pirfenidone analog enhances the therapeutic benefit of a selected amount of IP-10 when used in combination therapy for a disease, where the selected amount of pirfenidone or a pirfenidone analog provides no therapeutic benefit when used in monotherapy for the disease (2) regimens in which a selected amount of IP-10 enhances the therapeutic benefit of a selected amount of pirfenidone or a pirfenidone analog when used in combination therapy for a disease, where the selected amount of IP-10 provides no therapeutic benefit when used in monotherapy for the disease and (3) regimens in which a selected amount of IP-10 and a selected amount of pirfenidone or a pirfenidone analog provide a therapeutic benefit when used in combination therapy for a disease, where each of the selected amounts of IP-10 and pirfenidone or a pirfenidone analog, respectively, provides no therapeutic benefit when used in monotherapy for the disease. As used herein, a “synergistically effective amount” of IP-10 and pirfenidone or a pirfenidone analog, and its grammatical equivalents, shall be understood to include any regimen encompassed by any of (1)-(3) above.

IP-10 and Pirfenidone in Combination Therapy with Type I Interferon Receptor Agonist

In one aspect, the present invention provides combination therapy for the treatment of cancer, comprising administering effective amounts of IP-10, pirfenidone, and a Type I interferon receptor agonist. In some embodiments, the Type I interferon receptor agonist is IFN-α.

Effective dosages of IFN-α can range from 0.3 μg to 100 μg. Effective dosages of Infergen® consensus IFN-α can contain an amount of about 3 μg, about 9 μg, about 15 μg, about 18 μg, or about 27 μg, or about 30 μg of drug per dose. Effective dosages of IFN-α2a and IFN-α2b can contain an amount of about 3 million Units (MU) to about 30 MU of drug per dose. Effective dosages of PEGASYS®PEGylated IFN-α2a can contain an amount of about 5 μg to about 500 μg, or about 45 μg to about 450 μg, or about 60 μg to about 400 μg, or about 75 μg to about 350 μg, or about 90 μg to about 300 μg, about 105 μg to about 270 μg, or about 120 μg to about 240 μg, or about 135 μg to about 210 μg, or about 150 μg to about 180 μg, or about 135 μg, of drug per dose. Effective dosages of PEG-INTRON®PEGylated IFN-α2b can contain an amount of about 0.5 μg to about 5.0 μg, or about 0.75 μg to about 3.5 μg, or about 1.0 μg to about 3.0 μg, or about 1.25 μg to about 2.5 μg, or about 1.5 μg to about 2.0 μg, of drug per kg of body weight per dose. Effective dosages of PEGylated consensus interferon (PEG-CIFN) can contain an amount of about 9 μg to about 200 μg, or about 12 μg to about 180 μg, or about 15 μg to about 150 μg, or about 18 μg to about 120 μg, or about 21 μg to about 90 μg, or about 24 μg to about 75 μg, or about 27 μg to about 60 μg, or about 45 μg, of CIFN amino acid weight per dose of PEG-CIFN. Effective dosages of monoPEG (30 kD, linear)-ylated CIFN can contain an amount of about 5 μg to about 500 μg, or about or about 45 μg to about 450 μg, or about 60 μg to about 400 μg, or about 75 μg to about 350 μg, or about 90 μg to about 300 μg, about 105 μg to about 270 μg, or about 120 μg to about 240 μg, or about 135 μg to about 210 μg, or about 150 μg to about 180 μg, or about 135 μg, of drug per dose.

IFN-α is typically administered subcutaneously. For example, IFN-α can be administered subcutaneously qd, qod, tiw, or biw, or per day substantially continuously or continuously for a period of from about 2 weeks to about 52 weeks, from about 52 weeks to about 2 years, or longer.

In one embodiment, the invention provides a method using an effective amount of INFERGEN® consensus IFN-α, IP-10, and pirfenidone or a specific pirfenidone analog in the treatment of cancer in a patient comprising administering to the patient a dosage of INFERGEN® containing an amount of about 1 μg to about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IP-10, and pirfenidone or a specific pirfenidone analog in the treatment of cancer in a patient comprising administering to the patient a dosage of INFERGEN® containing an amount of about 3 μg to about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IP-10, and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IP-10, and pirfenidone or a specific pirfenidone analog in the treatment of cancer in a patient comprising administering to the patient a dosage of INFERGEN® containing an amount of about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of a consensus IFN-α, IP-10, and pirfenidone or a specific pirfenidone analog in the treatment of cancer in a patient comprising administering to the patient a dosage of PEGylated consensus IFN-α (PEG-CIFN) containing an amount of about 18 μg to about 90 μg, or about 27 μg to about 60 μg, or about 45 μg, of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of a consensus IFN-α, IP-10, and pirfenidone or a specific pirfenidone analog in the treatment of cancer in a patient comprising administering to the patient a dosage of monoPEG (30 kD, linear)-ylated consensus IFN-α containing an amount of about 5 μg to about 500 μg, or about or about 45 μg to about 450 μg, or about 60 μg to about 400 μg, or about 75 μg to about 350 μg, or abut 90 μg to about 300 μg, about 105 μg to about 270 μg, or about 120 μg to about 240 μg, or about 135 μg to about 210 μg, or about 150 μg to about 180 μg, or about 135 μg, of drug per dose, subcutaneously qw, qow, three times per month, or monthly, in combination with a dosage of pirfenidone or a specific pirfenidone analog containing an amount of about 50 mg to about 5,000 mg of drug orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IP-10, and pirfenidone or a specific pirfenidone analog in the treatment of cancer in a patient comprising administering to the patient a dosage of INFERGEN® containing an amount of about 5 μg to about 150 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IP-10, and pirfenidone or a specific pirfenidone analog in the treatment of cancer in a patient comprising administering to the patient a dosage of INFERGEN® containing an amount of about 5 μg to about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IP-10, and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

IP-10 and Pirfenidone in Combination Therapy with Type II Interferon Receptor Agonist

In another aspect, the present invention provides combination therapy for the treatment of cancer, comprising co-administering to the patient effective amounts of IP-10, pirfenidone, and a Type II interferon receptor agonist. In some embodiments, the Type II interferon receptor agonist is IFN-γ.

Effective dosages of IFN-γ range from about 0.5 μl/m² to about 500 μg/m², usually from about 1.5 μg/m² to 200 μg/m², depending on the size of the patient. This activity is based on 10⁶ international units (U) per 50 μg of protein. IFN-γ can be administered daily, every other day, three times a week, twice per week, or substantially continuously or continuously for a period of from about 2 weeks to about 52 weeks, from about 52 weeks to about 2 years, or longer.

In certain embodiments, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m^{m 2} to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m².

In one embodiment, the invention provides a method using an effective amount of IFN-γ, IP-10, and pirfenidone or a specific pirfenidone analog in the treatment of cancer in a patient comprising administering to the patient a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of IFN-γ, IP-10, and pirfenidone or a specific pirfenidone analog in the treatment of cancer in a patient comprising administering to the patient a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 100 mg to about 1,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of IFN-γ, IP-10, and pirfenidone or a specific pirfenidone analog in the treatment of cancer in a patient comprising administering to the patient a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In one embodiment, the invention provides a method using an effective amount of IFN-γ, IP-10, and pirfenidone or a specific pirfenidone analog in the treatment of cancer in a patient comprising administering to the patient a dosage of IFN-γ containing an amount of about 200 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

IP-10 and Pirfenidone in Combination Therapy with Type I Interferon Receptor Agonist and Type H Interferon Receptor Agonist

In one aspect, the present invention provides combination therapy for the treatment of cancer, comprising administering effective amounts of IP-10, pirfenidone, a Type I interferon receptor agonist, and a Type II interferon receptor agonist. In some embodiments, the Type I interferon receptor agonist is IFN-α. In other embodiments, the Type II interferon receptor agonist is IFN-γ. In still other embodiments, the Type I interferon receptor is IFN-α and the Type II interferon receptor agonist is IFN-γ.

IFN-α and IFN-γ are typically administered subcutaneously. For example, IFN-α and IFN-γ can be administered subcutaneously qd, qod, tiw, or biw, or per day substantially continuously or continuously for a period of from about 2 weeks to about 52 weeks, from about 52 weeks to about 2 years, or longer.

In one embodiment, the invention provides a method using an effective amount of INFERGEN® consensus IFN-α, IFN-γ, IP-10, and pirfenidone or a specific pirfenidone analog in the treatment of cancer in a patient comprising co-administering to the patient a dosage of INFERGEN® containing an amount of about 1 μg to about 30 μg of drug per dose of INFERGEN®, subcutaneously qd,.qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IFN-γ, IP-10, and pirfenidone or a specific pirfenidone analog in the treatment of cancer in a patient comprising co-administering to the patient a dosage of INFERGEN® containing an amount of about 3 μg to about 30 μg of drug per dose of INFERGEN®, subcutaneously qd, qod, tiw, or biw, or per day substantially continuously or continuously, and a dosage of IFN-γ containing an amount of about 30 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day-substantially continuously or continuously, for the desired treatment duration.

In another embodiment, the invention provides a method using an effective amount of INFERGEN® consensus IFN-α, IFN-γ, IP-10, and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IFN-γ, IP-10, and pirfenidone or a specific pirfenidone analog in the treatment of cancer in a patient comprising co-administering to the patient a dosage of INFERGEN® containing an amount of about 30 μg of drug per dose of INFERGEN®, subcutaneously qd, qod, tiw, or biw, or per day substantially continuously or continuously, and a dosage of IFN-γ containing an amount of about 200 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IFN-γ, IP-10, and pirfenidone or a specific pirfenidone analog in the treatment of cancer in a patient comprising co-administering to the patient a dosage of INFERGEN® containing an amount of about 5 μg to about 150 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IFN-γ, IP-10, and pirfenidone or a specific pirfenidone analog in the treatment of cancer in a patient comprising co-administering to the patient a dosage of INFERGEN® containing an amount of about 5 μg to about 45 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IFN-γ, IP-10, and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

IP-10 and Pirfenidone Combination Therapy as Adjuvant Therapy

In some embodiments, the present invention provides methods for combination therapy using IP-10 and pirfenidone, where the IP-10 and pirfenidone (or a pirfenidone analog) are administered as adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethyleneimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc.. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (M7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use in connection with the methods of the invention include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ (8) colony-stimulating factors; (9) inhibitors of angiogenesis; and (10) antagonists of tumor necrosis factor.

In one aspect, the invention contemplates the combination of IP-10 and pirfenidone or pirfenidone analog as an adjuvant to any therapy in which the cancer patient receives treatment with least one additional antineoplastic drug, where the additional drug is a tyrosine kinase inhibitor. In some embodiments, the tyrosine kinase inhibitor is a receptor tyrosine kinase (RTK) inhibitor, such as type I receptor tyrosine kinase inhibitors (e.g., inhibitors of epidermal growth factor receptors), type II receptor tyrosine kinase inhibitors (e.g., inhibitors of insulin receptor), type III receptor tyrosine kinase inhibitors (e.g., inhibitors of platelet-derived growth factor receptor), and type IV receptor tyrosine kinase inhibitors (e.g., fibroblast growth factor receptor). In other embodiments, the tyrosine kinase inhibitor is a non-receptor tyrosine kinase inhibitor, such as inhibitors of src kinases or janus kinases.

In another aspect, the invention contemplates the combination of IP-10 and pirfenidone or pirfenidone analog as an adjuvant to any therapy in which the cancer patient receives treatment with least one additional antineoplastic drug, where the additional drug is an inhibitor of a receptor tyrosine kinase involved in growth factor signaling pathway(s). In some embodiments, the inhibitor is genistein. In other embodiments, the inhibitor is an EGFR tyrosine kinase-specific antagonist, such as IRESSA™ gefitinib (ZD18398; Novartis), TARCEVA™ erolotinib (OSI-774; Roche; Genentech; OSI Pharmaceuticals), or tyrphostin AG1478 (4-(3-chloroanilino)-6,7-dimethoxyquinazoline. In still other embodiments, the inhibitor is any indolinone antagonist of Flk-1/KDR (VEGF-R2) tyrosine kinase activity described in U.S. Patent Application Publication No. 2002/0183364 A1, such as the indolinone antagonists of Flk-1/KDR (VEGF-R2) tyrosine kinase activity disclosed in Table 1 on pages 4-5 thereof. In further embodiments, the inhibitor is any of the substituted 3-[(4,5,6,7-tetrahydro-1H-indol-2-yl)methylene]-1,3-dihydroindol-2-one antagonists of Flk-1/KDR (VEGF-R2), FGF-R1 or PDGF-R tyrosine kinase activity disclosed in Sun, L., et al., J. Med. Chem., 43(14): 2655-2663 (2000). In additional embodiments, the inhibitor is any substituted 3-[(3- or 4-carboxyethylpyrrol-2-yl)methylidenyl]indolin-2-one antagonist of Flt-1 (VEGF-R1), Flk-1/KDR (VEGF-R2), FGF-R1 or PDGF-R tyrosine kinase activity disclosed in Sun, L., et al., J. Med. Chem., 42(25): 5120-5130 (1999).

In another aspect, the invention contemplates the combination of IP-10 and pirfenidone or pirfenidone analog as an adjuvant to any therapy in which the cancer patient receives treatment with least one additional antineoplastic drug, where the additional drug is an inhibitor of a non-receptor tyrosine kinase involved in growth factor signaling pathway(s). In some embodiments, the inhibitor is an antagonist of JAK2 tyrosine kinase activity, such as tyrphostin AG490 (2-cyano-3-(3,4-dihydroxyphenyl)-N-(benzyl)-2-propenamide). In other embodiments, the inhibitor is an antagonist of bcr-abl tyrosine kinase activity, such as GLEEVEC™ imatinib mesylate (STI-571; Novartis).

In another aspect, the invention contemplates the combination of IP-10 and pirfenidone or pirfenidone analog as an adjuvant to any therapy in which the cancer patient receives treatment with least one additional antineoplastic drug, where the additional drug is a serine/threonine kinase inhibitor. In some embodiments, the serine/threonine kinase inhibitor is a receptor serine/threonine kinase inhibitor, such as antagonists of TGF-β receptor serine/threonine kinase activity. In other embodiments, the serine/threonine kinase inhibitor is a non-receptor serine/threonine kinase inhibitor, such as antagonists of the serine/threonine kinase activity of the MAP kinases, protein kinase C (PKC), protein kinase A (PKA), or the cyclin-dependent kinases (CDKs).

In another aspect, the invention contemplates the combination of IP-10 and pirfenidone or pirfenidone analog as an adjuvant to any therapy in which the cancer patient receives treatment with least one additional antineoplastic drug, where the additional drug is an inhibitor of one or more kinases involved in cell cycle regulation. In some embodiments, the inhibitor is an antagonist of CDK2 activation, such as tryphostin AG490 (2-cyano-3-(3,4-dihydroxyphenyl)-N-(benzyl)-2-propenamide). In other embodiments, the inhibitor is an antagonist of CDK1/cyclin B activity, such as alsterpaullone. In still other embodiments, the inhibitor is an antagonist of CDK2 kinase activity, such as indirubin-3′-monoxime. In additional embodiments, the inhibitor is an AT? pool antagonist, such as lometrexol (described in U.S. Patent Application Publication No. 2002/0156023 A1).

In another aspect, the invention contemplates the combination of IP-10 and pirfenidone or pirfenidone analog as an adjuvant to any therapy in which the cancer patient receives treatment with least one additional antineoplastic drug, where the additional drug is an a tumor-associated antigen antagonist, such as an antibody antagonist. In some embodiments involving the treatment of HER2-expressing tumors, the tumor-associated antigen antagonist is an anti-HER2 monoclonal antibody, such as HERCEPTIN™ trastuzumab. In some embodiments involving the treatment of CD20-expressing tumors, such as B-cell lymphomas, the tumor-associated antigen antagonist is an anti-CD20 monoclonal antibody, such as RITUXAN™ rituximab.

In another aspect, the invention contemplates the combination of IP-10 and pirfenidone or pirfenidone analog as an adjuvant to any therapy in which the cancer patient receives treatment with least one additional antineoplastic drug, where the additional drug is a tumor growth factor antagonist. In some embodiments, the tumor growth factor antagonist is an antagonist of epidermal growth factor (EGF), such as an anti-EGF monoclonal antibody. In other embodiments, the tumor growth factor antagonist is an antagonist of epidermal growth factor receptor erbB1 (EGFR), such as an anti-EGFR monoclonal antibody inhibitor of EGFR activation or signal transduction.

In another aspect, the invention contemplates the combination of IP-10 and pirfenidone or pirfenidone analog as an adjuvant to any therapy in which the cancer patient receives treatment with least one additional antineoplastic drug, where the additional drug is an Apo-2 ligand agonist. In some embodiments, the Apo-2 ligand agonist is any of the Apo-2 ligand polypeptides described in WO 97/25428.

In another aspect, the invention contemplates the combination of IP-10 and pirfenidone or pirfenidone analog as an adjuvant to any therapy in which the cancer patient receives treatment with least one additional antineoplastic drug, where the additional drug is an anti-angiogenic agent. In some embodiments, the anti-angiogenic agent is a vascular endothelial cell growth factor (VEGF) antagonist, such as an anti-VEGF monoclonal antibody, e.g. AVASTIN™ bevacizumab (Genentech). In other embodiments, the anti-angiogenic agent is a retinoic acid receptor (RXR) ligand, such as any RXR ligand described in U.S. Patent Application Publication No. 2001/0036955 A1 or in any of U.S. Pat. Nos. 5,824,685; 5,780,676; 5,399,586; 5,466,861; 4,810,804; 5,770,378; 5,770,383; or 5,770,382. In still other embodiments, the anti-angiogenic agent is a peroxisome proliferator-activated receptor (PPAR) gamma ligand, such as any PPAR gamma ligand described in U.S. Patent Application Publication No. 2001/0036955 A1.

Determining Susceptibility of a Tumor to IP-10 and Pirfenidone Combination Therapy

The present invention further provides methods for determining the susceptibility or sensitivity of a tumor to growth inhibition by IP-10 and pirfenidone combination therapy. The methods generally involve culturing a patient's tumor cell in vitro in a medium (e.g., a liquid culture medium) comprising IP-10 and pirfenidone; and determining the effect, if any, of IP-10 and pirfenidone on the survival of the cell. A reduction in the survival of the cell, compared with the survival in the absence of IP-10 and pirfenidone, indicates that the tumor is susceptible to treatment with IP-10 and pirfenidone combination therapy.

For example, a reduction of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, in the cell survival when cultured in the presence of IP-10 and pirfenidone, compared with the cell survival in the absence of IP-10 and pirfenidone, indicates that the tumor is susceptible to treatment with IP-10 and pirfenidone combination therapy.

Sensitivity of a tumor cell to IP-10 plus pirfenidone treatment is determined using any known method. Typically, a biopsy sample is obtained using standard procedures, and cells from the biopsied tissue are cultured in vitro. The method generally involves culturing cells from the biopsied tissue in vitro in the presence of IP-10 and pirfenidone, and, after a suitable time, determining the number of live cells in the culture, compared to the number of live cells in a culture not treated with IP-10 and pirfenidone. Live cells can be distinguished from dead cells using any standard assay method, including, but not limited to, a trypan blue dye exclusion assay; an MTT (3-(4,5-dimethylthiazol-2-yl)-2,5-diphenyl-2H-tetrazolium bromide) assay; a flow cytometric assay that relies upon exclusion of a dye from live, but not dead cells, e.g., propidium iodide uptake (where propidium iodide is taken up by dead, but not live cells), uptake of a Hoechst dye, such as Hoechst 33342, that enters live, but not dead cells, and the like, which assays are used in conjunction with fluorescence activated cell sorter to distinguish live from dead cells. For example, survival is determined using a method as described in the Example.

In some embodiments, the invention provides methods of treating cancer in an individual having a cancer susceptible to treatment with IP-10 and pirfenidone, the method comprising determining the susceptibility of the cancer to treatment with IP-10 and pirfenidone; and administering an effective amount of IP-10 and pirfenidone to the individual.

Type I Interferon Receptor Agonist and Pirfenidone or a Pirfenidone Analog Combination Therapy

In some embodiments, pirfenidone or a pirfenidone analog is administered during the entire course of Type I interferon receptor agonist treatment. In other embodiments, pirfenidone or a pirfenidone analog is administered for a period of time that is overlapping with that of the Type I interferon receptor agonist treatment, e.g., the pirfenidone or pirfenidone analog treatment can begin before the Type I interferon receptor agonist treatment begins and end before the Type I interferon receptor agonist treatment ends; the pirfenidone or pirfenidone analog treatment can begin after the Type I interferon receptor agonist treatment begins and end after the Type I interferon receptor agonist treatment ends; the pirfenidone or pirfenidone analog treatment can begin after the Type I interferon receptor agonist treatment begins and end before the Type I interferon receptor agonist treatment ends; or the pirfenidone or pirfenidone analog treatment can begin before the Type I interferon receptor agonist treatment begins and end after the Type I interferon receptor agonist treatment ends.

Effective dosages of IFN-α can range from 0.3 μg to 100 μg. Effective dosages of Infergen® consensus IFN-α can contain an amount of about 3 μg, about 9 μg, about 15 μg, about 18 μg, or about 27 μg, or about 30 μg of drug per dose. Effective dosages of IFN-α2a and IFN-α2b can contain an amount of about 3 million Units (MU) to about 30 MU of drug per dose. Effective dosages of PEGASYS®PEGylated IFN-α2a can contain an amount of about 5 μg to about 500 μg, or about 45 μg to about 450 μg, or about 60 μg to about 400 μg, or about 75 μg to about 350 μg, or about 90 μg to about 300 μg, about 105 μg to about 270 μg, or about 120 μg to about 240 μg, or about 135 μg to about 210 μg, or about 150 μg to about 180 μg, or about 135 μg, of drug per dose. Effective dosages of PEG-INTRON®PEGylated IFN-α2b can contain an amount of about 0.5 μg to about 5.0 μg, or about 0.75 μg to about 3.5 μg, or about 1.0 μg to about 3.0 μg, or about 1.25 μg to about 2.5 μg, or about 1.5 μg to about 2.0 μg, of drug per kg of body weight per dose. Effective dosages of PEGylated consensus interferon (PEG-CIFN) can contain an amount of about 9 μg to about 200 μg, or about 12 μg to about 180 μg, or about 15 μg to about 150 μg, or about 18 μg to about 120 μg, or about 21 μg to about 90 μg, or about 24 μg to about 75 μg, or about 27 μg to about 60 μg, or about 45 μg, of CIFN amino acid weight per dose of PEG-CIFN. Effective dosages of monoPEG (30 kD, linear)-ylated CIFN can contain an amount of about 5 μg to about 500 μg, or about or about 45 μg to about 450 μg, or about 60 μg to about 400 μg, or about 75 μg to about 350 μg, or about 90 μg to about 300 μg, about 105 μg to about 270 μg, or about 120 μg to about 240 μg, or about 135 μg to about 210 μg, or about 150 μg to about 180 μg, or about 135 μg, of drug per dose.

IFN-α is typically administered subcutaneously. For example, IFN-α can be administered subcutaneously qd, qod, tiw, or biw, or per day substantially continuously or continuously for a period of from about 2 weeks to about 52 weeks, from about 52 weeks to about 2 years, or longer.

Those of skill will readily appreciate that dose levels can vary as a function of the specific agent used (e.g., the specific form of IFN-α), the severity of the symptoms and the susceptibility of the subject to side effects. Preferred dosages for a given agent are readily determinable by those of skill in the art by a variety of means.

In many embodiments, multiple doses of IFN-α are administered. For example, an interferon receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In general, effective dosages of pirfenidone or specific pirfenidone analogs can range from about 0.5 mg/kg/day to about 200 mg/kg/day, or at a fixed dosage of about 400 mg to about 3600 mg per day, or about 50 mg to about 10,000 mg per day, or about 100 mg to about 1,000 mg per day, or about 1,000 mg to about 3,000 mg per day, or about 1,000 mg to about 10,000 mg per day, administered orally, optionally in two or more divided doses per day. Other doses and formulations of pirfenidone and pirfenidone analogs suitable for use in a subject method for the treatment of cancer are described in U.S. Pat. Nos. 3,974,281; 3,839,346; 4,042,699; 4,052,509; 5,310,562; 5,518,729; 5,716,632; and 6,090,822.

Those of skill in the art will readily appreciate that dose levels of pirfenidone or pirfenidone analog can vary as a function of the specific compound, the severity of the symptoms and the susceptibility of the subject to side effects. Preferred dosages for a given compound are readily determinable by those of skill in the art by a variety of means.

Pirfenidone (or a pirfenidone analog) can be administered daily, twice a day, or three times a day, or in divided daily doses ranging from 2 to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

IFN-α and pirfenidone (or pirfenidone analog) are generally administered in separate formulations. IFN-α and pirfenidone (or pirfenidone analog) may be administered substantially simultaneously, or within about 30 minutes, about 1 hour, about 2 hours, about 4 hours, about 8 hours, about 16 hours, about 24 hours, about 36 hours, about 72 hours, about 4, days, about 7 days, or about 2 weeks of one another.

In one embodiment, the invention provides a method using an effective amount of INFERGEN® consensus IFN-α, and pirfenidone or a specific pirfenidone analog in the treatment of cancer in a patient comprising administering to the patient a dosage of INFERGEN® containing an amount of about 1 μg to about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 3 μg to about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, for the desired treatment duration.

In another embodiment, the invention provides a method using an effective amount of INFERGENαconsensus IFN-α and pirfenidone or a specific pirfenidone analog in the treatment of cancer in a patient comprising administering to the patient a dosage of INFERGEN® containing an amount of about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, for the desired treatment duration.

In another embodiment, the invention provides a method using an effective amount of a consensus IFN-α and pirfenidone or a specific pirfenidone analog in the treatment of cancer in a patient comprising administering to the patient a dosage of PEGylated consensus IFN-α (PEG-CIFN) containing an amount of about 18 μg to about 90 μg, or about 27 μg to about 60 μg, or about 45 μg, of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose orally qd, optionally in two or more divided doses per day, for the desired treatment duration.

In another embodiment, the invention provides a method using an effective amount of a consensus IFN-α and pirfenidone or a specific pirfenidone analog in the treatment of cancer in a patient comprising administering to the patient a dosage of PEGylated consensus IFN-α (PEG-CIFN) containing an amount of about 10 μg to about 150 μg of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose orally qd, optionally in two or more divided doses per day, for the desired treatment duration.

In another embodiment, the invention provides a method using an effective amount of a consensus IFN-α and pirfenidone or a specific pirfenidone analog in the treatment of cancer in a patient comprising administering to the patient a dosage of monoPEG (30 kD, linear)-ylated consensus IFN-α containing an amount of about 5 μg to about 500 μg, or about or about 45 μg to about 450 μg, or about 60 μg to about 400 μg, or about 75 μg to about 350 μg, or abut 90 μg to about 300 μg, about 105 μg to about 270 μg, or about 120 μg to about 240 μg, or about 135 μg to about 210 μg, or about 150 μg to about 180 μg, or about 135 μg, of drug per dose, subcutaneously qw, qow, three times per month, or monthly, in combination with a dosage of pirfenidone or a specific pirfenidone analog containing an amount of about 50 mg to about 5,000 mg of drug orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 5 μg to about 150 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 5 μg to about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, for the desired treatment duration.

Synergistic Combinations of a Type I Interferon Receptor Agonist and Pirfenidone

In many embodiments, the effective amounts of a Type I interferon receptor agonist and pirfenidone (or a pirfenidone analog) are synergistic amounts. As used herein, a “synergistic combination” or a “synergistic amount” of a Type I interferon receptor agonist and pirfenidone or a pirfenidone analog is a combined dosage that is more effective in the therapeutic or prophylactic treatment of cancer than the incremental improvement in treatment outcome that could be predicted or expected from a merely additive combination of (i) the therapeutic or prophylactic benefit of the Type I interferon receptor agonist when administered at that same dosage as a monotherapy and (ii) the therapeutic or prophylactic benefit of pirfenidone or a pirfenidone analog when administered at the same dosage as a monotherapy.

In some embodiments of the invention,a selected amount of a Type I interferon receptor agonist and a selected amount of pirfenidone or a pirfenidone analog are effective when used in combination therapy for a disease, but the selected amount of Type I interferon receptor agonist and/or the selected amount of pirfenidone or a pirfenidone analog is ineffective when used in monotherapy for the disease. Thus, the invention encompasses (1) regimens in which a selected amount of pirfenidone or a pirfenidone analog enhances the therapeutic benefit of a selected amount of Type I interferon receptor agonist when used in combination therapy for a disease, where the selected amount of pirfenidone or a pirfenidone analog provides no therapeutic benefit when used in monotherapy for the disease (2) regimens in which a selected amount of Type I interferon receptor agonist enhances the therapeutic benefit of a selected amount of pirfenidone or a pirfenidone analog when used in combination therapy for a disease, where the selected amount of Type I interferon receptor agonist provides no therapeutic benefit when used in monotherapy for the disease and (3) regimens in which a selected amount of Type I interferon receptor agonist and a selected amount of pirfenidone or a pirfenidone analog provide a therapeutic benefit when used in combination therapy for a disease, where each of the selected amounts of Type I interferon receptor agonist and pirfenidone or a pirfenidone analog, respectively, provides no therapeutic benefit when used in monotherapy for the disease. As used herein, a “synergistically effective amount” of Type I interferon receptor agonist and pirfenidone or a pirfenidone analog, and its grammatical equivalents, shall be understood to include any regimen encompassed by any of (1)-(3) above.

Type I Interferon Receptor Agonist and Pirfenidone in Combination Therapy with IP-10

In one aspect, the present invention provides combination therapy for the treatment of cancer, comprising administering effective amounts of a Type I interferon receptor agonist, pirfenidone, and IP-10. In some embodiments, the Type I interferon receptor agonist is IFN-α.

Effective dosages of IP-10 range from about 0.1 μg to about 1000 μg per dose, e.g., from about 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 η per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, or from about 900 μg to about 1000 μg per dose.

In some embodiments, effective dosages of IP-10 are expressed as mg/kg body weight. In these embodiments, effective dosages of IP-10 are from about 0.1 mg/kg body weight to about 10 mg/kg body weight, e.g., from about 0.1 mg/kg body weight to about 0.5 mg/kg body weight, from about 0.5 mg/kg body weight to about 1.0 mg/kg body weight, from about 1.0 mg/kg body weight to about 2.5 mg/kg body weight, from about 2.5 mg/kg body weight to about 5.0 mg/kg body weight, from about 5.0 mg/kg body weight to about 7.5 mg/kg body weight, or from about 7.5 mg/kg body weight to about 10 mg/kg body weight.

In many embodiments, IP-10 is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The IP-10 can be administered tid, bid, qd, qod, biw, tiw, qw, qow, three times per month, once monthly, substantially continuously, or continuously.

Those of skill will readily appreciate that dose levels can vary as a function of the specific agent used (e.g., the specific form of IP-10), the severity of the symptoms and the susceptibility of the subject to side effects. Preferred dosages for a given agent are readily determinable by those of skill in the art by a variety of means.

In many embodiments, multiple doses of IP-10 are administered. For example, an interferon receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In one embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 1 μg to about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 3 μg to about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of a consensus IFN-α, IP-10, and pirfenidone or a specific pirfenidone analog in the treatment of cancer in a patient comprising administering to the patient a dosage of PEGylated consensus IFN-α (PEG-CIFN) containing an amount of about 10 μg to about 150 μg of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of a consensus IFN-α, IP-10, and pirfenidone or a specific pirfenidone analog in the treatment of cancer in a patient comprising administering to the patient a dosage of monoPEG (30 kD, linear)-ylated consensus IFN-α containing an amount of about 5 μg to about 500 μg, or about 45 μg to about 450 μg, or about 60 μg to about 400 μg, or about 75 μg to about 350 μg, or about 90 μg to about 300 μg, or about 150 μg to about 270 μg, or about 105 μg to about 270 μg, or about 120 μg to about 240 μg, or about 135 μg to about 210 μg, or about 150 μg to about 180 μg, or about 135 μg, of drug per dose, subcutaneously qw, qow, three times per month, or monthly, in combination with a dosage of pirfenidone or a specific pirfenidone analog containing an amount of about 50 mg to about 5,000 mg of drug orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 5 μg to about 150 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 5 μg to about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

Type I Interferon Receptor Agonist and Pirfenidone in Combination Therapy with Type II Interferon Receptor Agonist

In another aspect, the present invention provides combination therapy for the treatment of cancer, comprising co-administering to the patient effective amounts of a Type I interferon receptor agonist, pirfenidone, and a Type II interferon receptor agonist. In some embodiments, the Type II interferon receptor agonist is IFN-γ.

Effective dosages of IFN-γ range from about 0.5 μg/m² to about 500 μg/m², usually from about 1.5 μg/m² to 200 μg/m², depending on the size of the patient. This activity is based on 10⁶ international units (U) per 50 μg of protein. IFN-γ can be administered daily, every other day, three times a week, twice per week, or substantially continuously or continuously for a period of from about 2 weeks to about 52 weeks, from about 52 weeks to about 2 years, or longer.

In certain embodiments, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μ/m² to about 150 μg/m². In some embodiments, the dosage groups range from about 25 μg/m² to about 100 μg/m². In other embodiments, the dosage groups range from about 25 μg/m² to about 50 μg/m².

In one embodiment, the invention provides a method using an effective amount of INFERGEN®consensus IFN-α, IFN-γ, and pirfenidone or a specific pirfenidone analog in the treatment of cancer in a patient comprising co-administering to the patient a dosage of INFERGEN® containing an amount of about 1 μg to about 30 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising co-administering to the patient a dosage of INFERGEN® containing an amount of about 3 μg to about 30 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising co-administering to the patient a dosage of INFERGEN® containing an amount of about 30 μg of drug per dose of INFERGEN®, subcutaneously qd, qod, tiw, or biw, or per day substantially continuously or continuously, and a dosage of IFN-γ containing an amount of about 200 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising co-administering to the patient a dosage of INFERGEN® containing an amount of about 5 μg to about 150 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising co-administering to the patient a dosage of INFERGEN® containing an amount of about 5 μg to about 45 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, for the desired treatment duration.

Type I Interferon Receptor Agonist and Pirfenidone in Combination Therapy with IP-10 and Type II Interferon Receptor Agonist

In one aspect, the present invention provides combination therapy for the treatment of cancer, comprising administering effective amounts of IP-10, pirfenidone, a Type I interferon receptor agonist, and a Type II interferon receptor agonist. In some embodiments, the Type I interferon receptor agonist is IFN-α. In other embodiments, the Type II interferon receptor agonist is IFN-γ. In still other embodiments, the Type I interferon receptor is IFN-α and the Type II interferon receptor agonist is IFN-γ.

IFN-α and IFN-γ are typically administered subcutaneously. For example,. IFN-α and IFN-γ can be administered subcutaneously qd, qod, tiw, or biw, or per day substantially continuously or continuously for a period of from about 2 weeks to about 52 weeks, from about 52 weeks to about 2 years, or longer.

In one embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 1 μg to about 30 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 3 μg to about 30 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 30 μg of drug per dose of INFERGEN®, subcutaneously qd, qod, tiw, or biw, or per day substantially continuously or continuously, and a dosage of IFN-γ containing an amount of about 200 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 5 μg to about 150 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, and a dosage of IP-10 containing an amount of from about 0.1 782 g to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 5 μg to about 45 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

Type I Interferon Receptor Agonist and Pirfenidone Combination Therapy as Adjuvant Therapy

In some embodiments, the present invention provides methods for combination therapy using a Type I interferon receptor agonist and pirfenidone, where the Type I interferon receptor agonist and pirfenidone are administered as adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine; and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17a-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc.. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (M7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use in connection with the methods of the invention include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ (8) colony-stimulating factors; (9) inhibitors of angiogenesis; and (10) antagonists of tumor necrosis factor.

In one aspect, the invention contemplates the combination of a Type I interferon receptor agonist and pirfenidone or pirfenidone analog as an adjuvant to any therapy in which the cancer patient receives treatment with least one additional antineoplastic drug, where the additional drug is a tyrosine kinase inhibitor. In some embodiments, the tyrosine kinase inhibitor is a receptor tyrosine kinase (RTK) inhibitor, such as type I receptor tyrosine kinase inhibitors (e.g., inhibitors of epidermal growth factor receptors), type II receptor tyrosine kinase inhibitors (e.g., inhibitors of insulin receptor), type III receptor tyrosine kinase inhibitors (e.g., inhibitors of platelet-derived growth factor receptor), and type IV receptor tyrosine kinase inhibitors (e.g., fibroblast growth factor receptor). In other embodiments, the tyrosine kinase inhibitor is a non-receptor tyrosine kinase inhibitor, such as inhibitors of src kinases or janus kinases.

In another aspect, the invention contemplates the combination of a Type I interferon receptor agonist and pirfenidone or pirfenidone analog as an adjuvant to any therapy in which the cancer patient receives treatment with least one additional antineoplastic drug, where the additional drug is an inhibitor of a receptor tyrosine kinase involved in growth factor signaling pathway(s). In some embodiments, the inhibitor is genistein. In other embodiments, the inhibitor is an EGFR tyrosine kinase-specific antagonist, such as IRESSA™ gefitinib (ZD18398; Novartis), TARCEVA™ erolotinib (OSI-774; Roche; Genentech; OSI Pharmaceuticals), or tyrphostin AG1478 (4-(3-chloroanilino)-6,7-dimethoxyquinazoline. In still other embodiments, the inhibitor is any indolinone antagonist of Flk-1/KDR (VEGF-R2) tyrosine kinase activity described in U.S. Patent Application Publication No. 2002/0183364 A1, such as the indolinone antagonists of Flk-1/KDR (VEGF-R2) tyrosine kinase activity disclosed in Table 1 on pages 4-5 thereof In further embodiments, the inhibitor is any of the substituted 3-[(4,5,6,7-tetrahydro-1H-indol-2-yl)methylene]-1,3-dihydroindol-2-one antagonists of Flk-1/KDR (VEGF-R2), FGF-R1 or PDGF-R tyrosine kinase activity disclosed in Sun, L., et al., J. Med. Chem., 43(14): 2655-2663 (2000). In additional embodiments, the inhibitor is any substituted 3-[(3- or 4-carboxyethylpyrrol-2-yl)methylidenyl]indolin-2-one antagonist of Flt-1 (VEGF-R1), Flk-1/KDR (VEGF-R2), FGF-R1 or PDGF-R tyrosine kinase activity disclosed in Sun, L., et al., J. Med. Chem.. 42(25): 5120-5130 (1999).

In another aspect, the invention contemplates the combination of a Type I interferon receptor agonist and pirfenidone or pirfenidone analog as an adjuvant to any therapy in which the cancer patient receives treatment with least one additional antineoplastic drug, where the additional drug is an inhibitor of a non-receptor tyrosine kinase involved in growth factor signaling pathway(s). In some embodiments, the inhibitor is an antagonist of JAK2 tyrosine kinase activity, such as tyrphostin AG490 (2-cyano-3-(3,4-dihydroxyphenyl)-N-(benzyl)-2-propenamide). In other embodiments, the inhibitor is an antagonist of bcr-abl tyrosine kinase activity, such as GLEEVEC™ imatinib mesylate (STI-571; Novartis).

In another aspect, the invention contemplates the combination of a Type I interferon receptor agonist and pirfenidone or pirfenidone analog as an adjuvant to any therapy in which the cancer patient receives treatment with least one additional antineoplastic drug, where the additional drug is a serine/threonine kinase inhibitor. In some embodiments, the serine/threonine kinase inhibitor is a receptor serine/threonine kinase inhibitor, such as antagonists of TGF-β receptor serine/threonine kinase activity. In other embodiments, the serine/threonine kinase inhibitor is a non-receptor serine/threonine kinase inhibitor, such as antagonists of the serine/threonine kinase activity of the MAP kinases, protein kinase C (PKC), protein kinase A (PKA), or the cyclin-dependent kinases (CDKs).

In another aspect, the invention contemplates the combination of a Type I interferon receptor agonist and pirfenidone or pirfenidone analog as an adjuvant to any therapy in which the cancer patient receives treatment with least one additional antineoplastic drug, where the additional drug is an inhibitor of one or more kinases involved in cell cycle regulation. In some embodiments, the inhibitor is an antagonist of CDK2 activation, such as tryphostin AG490 (2-cyano-3-(3,4-dihydroxyphenyl)-N-(benzyl)-2-propenamide). In other embodiments, the inhibitor is an antagonist of CDK1/cyclin B activity, such as alsterpaullone. In still other embodiments, the inhibitor is an antagonist of CDK2 kinase activity, such as indirubin-3′-monoxime. In additional embodiments, the inhibitor is an ATP pool antagonist, such as lometrexol (described in U.S. Patent Application Publication No. 2002/0156023 A1).

In another aspect, the invention contemplates the combination of a Type I interferon receptor agonist and pirfenidone or pirfenidone analog as an adjuvant to any therapy in which the cancer patient receives treatment with least one additional antineoplastic drug, where the additional drug is an a tumor-associated antigen antagonist, such as an antibody antagonist. In some embodiments involving the treatment of HER2-expressing tumors, the tumor-associated antigen antagonist is an anti-HER2 monoclonal antibody, such as HERCEPTIN™ trastuzumab. In some embodiments involving the treatment of CD20-expressing tumors, such as B-cell lymphomas, the tumor-associated antigen antagonist is an anti-CD20 monoclonal antibody, such as RITUXAN™ rituximab.

In another aspect, the invention contemplates the combination of a Type I interferon receptor agonist and pirfenidone or pirfenidone analog as an adjuvant to any therapy in which the cancer patient receives treatment with least one additional antineoplastic drug, where the additional drug is a tumor growth factor antagonist. In some embodiments, the tumor growth factor antagonist is an antagonist of epidermal growth factor (EGF), such as an anti-EGF monoclonal antibody. In other embodiments, the tumor growth factor antagonist is an antagonist of epidermal growth factor receptor erbB1 (EGFR), such as an anti-EGFR monoclonal antibody inhibitor of EGFR activation or signal transduction.

In another aspect, the invention contemplates the combination of a Type I interferon receptor agonist and pirfenidone or pirfenidone analog as an adjuvant to any therapy in which the cancer patient receives treatment with least one additional antineoplastic drug, where the additional drug is an Apo-2 ligand agonist. In some embodiments, the Apo-2 ligand agonist is any of the Apo-2 ligand polypeptides described in WO 97/25428.

In another aspect, the invention contemplates the combination of a Type I interferon receptor agonist and pirfenidone or pirfenidone analog as an adjuvant to any therapy in which the cancer patient receives treatment with least one additional antineoplastic drug, where the additional drug is an anti-angiogenic agent. In some embodiments, the anti-angiogenic agent is a vascular endothelial cell growth factor (VEGF) antagonist, such as an anti-VEGF monoclonal antibody, e.g. AVASTIN™ bevacizumab (Genentech). In other embodiments, the anti-angiogenic agent is a retinoic acid receptor (RXR) ligand, such as any RXR ligand described in U.S. Patent Application Publication No. 2001/0036955 A1 or in any of U.S. Pat. Nos. 5,824,685; 5,780,676; 5,399,586; 5,466,861; 4,810,804; 5,770,378; 5,770,383; or 5,770,382. In still other embodiments, the anti-angiogenic agent is a peroxisome proliferator-activated receptor (PPAR) gamma ligand, such as any PPAR gamma ligand described in U.S. Patent Application Publication No. 2001/0036955 A1.

Determining Susceptibility of Tumor to Type I Interferon Receptor Agonist and Pirfenidone Combination Therapy

The present invention further provides methods for determining the susceptibility or sensitivity of a tumor to growth inhibition by Type I interferon receptor agonist and pirfenidone combination therapy. The methods generally involve culturing a patient's tumor cell in vitro in a medium comprising a Type I interferon receptor agonist and pirfenidone; and determining the effect, if any, of the Type I interferon receptor agonist and pirfenidone on the survival of the cell. A reduction in the survival of the cell, compared with the survival in the absence of Type I interferon receptor agonist and pirfenidone, indicates that the tumor is susceptible to treatment with Type I interferon receptor agonist and pirfenidone combination therapy.

For example, a reduction of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, in the cell survival when cultured in the presence of a Type I interferon receptor agonist and pirfenidone, compared with the cell survival in the absence of the Type I interferon receptor agonist and pirfenidone, indicates that the tumor is susceptible to treatment with the Type I interferon receptor agonist and pirfenidone combination therapy.

Sensitivity of a tumor cell to Type I interferon receptor agonist plus pirfenidone treatment is determined using any known method. Typically, a biopsy sample is obtained using standard procedures, and cell from the biopsied tissue are cultured in vitro. The method generally involves culturing cells from the biopsied tissue in vitro in the presence of Type I interferon receptor agonist and pirfenidone, and, after a suitable time, determining the number of live cells in the culture, compared to the number of live cells in a culture not treated with Type I interferon receptor agonist and pirfenidone. Live cells can be distinguished from dead cells using any standard assay method, including, but not limited to, a trypan blue dye exclusion assay; an Mu (3-4,5-dimethylthiazol-2-yl)2,5diphenyl-2H-tetrazolium bromide) assay; a flow cytometric assay that relies upon exclusion of a dye from live, but not dead cells, e.g., propidium iodide uptake (where propidium iodide is taken up by dead, but not live cells), uptake of a Hoechst dye, such as Hoechst 33342, that enters live, but not dead cells, and the like, which assays are used in conjunction with fluorescence activated cell sorter to distinguish live from dead cells. For example; survival-is-determined using a method as described in the Example.

In some embodiments, the invention provides methods of treating cancer in an individual having a cancer susceptible to treatment with Type I interferon receptor agonist and pirfenidone, the method comprising determining the susceptibility of the cancer to treatment with Type I interferon receptor agonist and pirfenidone; and administering an effective amount of Type I interferon receptor agonist and pirfenidone to the individual.

Type I Interferon Receptor Agonist and IP-10 Combination Therapy

In some embodiments, IP-10 is administered during the entire course of Type I interferon receptor agonist treatment. In other embodiments, IP-10 is administered for a period of time that is overlapping with that of the Type I interferon receptor agonist treatment, e.g., the IP-10 treatment can begin before the Type I interferon receptor agonist treatment begins and end before the Type I interferon receptor agonist treatment ends; the IP-10 treatment can begin after the Type I interferon receptor agonist treatment begins and end after the Type I interferon receptor agonist treatment ends; the IP-10 treatment can begin after the Type I interferon receptor agonist treatment begins and end before the Type I interferon receptor agonist treatment ends; or the IP-10 treatment can begin before the Type I interferon receptor agonist treatment begins and end after the Type I interferon receptor agonist treatment ends.

Effective dosages of IFN-α can range from 0.3 μg to 100 μg. Effective dosages of Infergen® consensus IFN-α can contain an amount of about 3 μg, about 9 μg, about 15 μg, about 18 μg, or about 27 μg, or about 30 μg of drug per dose. Effective dosages of IFN-α2a and IFN-α2b can contain an amount of about 3 million Units (MU) to about 30 MU of drug per dose. Effective dosages of PEGASYS®PEGylated IFN-α2a can contain an amount of about 5 μg to about 500 μg, or about 45 μg to about 450 μg, or about 60 μg to about 400 μg, or about 75 μg to about 350 μg, or about 90 μg to about 300 μg, about 105 μg to about 270 μg, or about 120 μg to about 240 μg, or about 135 μg to about 210 μg, or about 150 μg to about 180 μg, or about 135 μg, of drug per dose. Effective dosages of PEG-INTRON®PEGylated IFN-α2b can contain an amount of about 0.5 μg to about 5.0 μg, or about 0.75 μg to about 3.5 μg or about 1.0 μ to about 3.0 μg, or about 1.25 μg to about 2.5 μg, or about 1.5 μg to about 2.0 μg, of drug per kg of body weight per dose. Effective dosages of PEGylated consensus interferon (PEG-CIFN) can contain an amount of about 9 μg to about 200 μg, or about 12 μg to about 180 μg, or about 15 μg to about 150 μg, or about 18 μg to about 120 μg, or about 21 μg to about 90 μg, or about 24 μg to about 75 μg, or about 27 μg to about 60 μg, or about 45 μg, of CIFN amino acid weight per dose of PEG-CIFN. Effective dosages of monoPEG (30 kD, linear)-ylated CIFN can contain an amount of about 5 μg to about 500 μg, or about or about 45 μg to about 450 μg, or about 60 μg to about 400 μg, or about 75 μg to about 350 μg, or about 90 μg to about 300 μg, about 105 μg to about 270 μg, or about 120 μg to about 240 μg, or about 135 μg to about 210 μg, or about 150 μg to about 180 μg, or about 135 μg, of drug per dose.

IFN-α is typically administered subcutaneously. For example, IFN-α can be administered subcutaneously qd, qod, tiw, or biw, or per day substantially continuously or continuously for a period of from about 2 weeks to about 52 weeks, from about 52 weeks to about 2 years, or longer.

Those of skill will readily appreciate that dose levels can vary as a function of the specific agent used (e.g., the specific form of IFN-α), the severity of the symptoms and the susceptibility of the subject to side effects. Preferred dosages for a given agent are readily determinable by those of skill in the art by a variety of means.

In many embodiments, multiple doses of IFN-α are administered. For example, an interferon receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Effective dosages of IP-10 range from 0.1 μg to 1000 μg per dose, e.g., from about 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μ per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, or from about 900 μg to about a 1000 μg per dose.

In some embodiments, effective dosages of IP-10 are expressed as mg/kg body weight. In these embodiments, effective dosages of IP-10 are from about 0.1 mg/kg body weight to about 10 mg/kg body weight, e.g., from about 0.1 mg/kg body weight to about 0.5 mg/kg body weight, from about 0.5 mg/kg body weight to about 1.0 mg/kg body weight, from about 1.0 mg/kg body weight to about 2.5 mg/kg body weight, from about 2.5 mg/kg body weight to about 5.0 mg/kg body weight, from about 5.0 mg/kg body weight to about 7.5 mg/kg body weight, or from about 7.5 mg/kg body weight to about 10 mg/kg body weight.

In many embodiments, IP-10 is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The IP-10 can be administered tid, bid, qd, qod, biw, tiw, qw, qow, three times per month, once monthly, substantially continuously, or continuously.

Those of skill will readily appreciate that dose levels can vary as a function of the specific agent used (e.g., the specific form of IP-10), the severity of the symptoms and the susceptibility of the subject to side effects. Preferred dosages for a given agent are readily determinable by those of skill in the art by a variety of means.

In many embodiments, multiple doses of IP-10 are administered. For example, IP-10 is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

IFN-α and IP-10 are generally administered in separate formulations. IFN-α and IP-10 may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

In one embodiment, the invention provides a-method using an effective amount of INFERGEN®consensus IFN-α, and IP-10 in the treatment of cancer in a patient comprising administering to the patient a dosage of INFERGEN® containing an amount of about 1 μg to about 30 μg of drug per dose of INFERGEN®, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α and IP-10 in the treatment of cancer in a patient comprising administering to the patient a dosage of INFERGEN® containing an amount of about 3 μg to about 30 μg of drug per dose of INFERGEN®, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α and IP-10 in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α and IP-10 in the treatment of cancer in a patient comprising administering to the patient a dosage of INFERGEN®D containing an amount of about 30 μg of drug per dose of INFERGEN®, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of a consensus IFN-α and IP-10 in the treatment of cancer in a patient comprising administering to the patient a dosage of PEGylated consensus IFN-α (PEG-CIFN) containing an amount of about 10 μg to about 150 μg of CIFN amino acid weight per dose of PEG-CIFN, subcutaneously qw, qow, three times per month, or monthly, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, optionally in two or more divided doses per day, for the desired treatment duration.

In another embodiment, the invention provides a method using an effective amount of a consensus IFN-α and IP-10 in the treatment of cancer in a patient comprising administering to the patient a dosage of monoPEG (30 kD, linear)-ylated consensus IFN-α containing an amount of about 5 μg to about 500 μg, or about or about 45 μg to about 450 μg, or about 60 μg to about 400 μg, or about 75 μg to about 350 μg, or abut 90 μg to about 300 μg, about 105 μg to about 270 μg, or about 120 μg to about 240 μg, or about 135 μg to about 210 μg, or about 150 μg to about 180 μg, or about 135 μg, of drug per dose, subcutaneously qw, qow, three times per month, or monthly, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α, and IP-10 in the treatment of cancer in a patient comprising administering to the patient a dosage of INFERGEN® containing an amount of about 5 μg to about 150 μg of drug per dose of INFERGEN®, subcutaneously qd, qod, tiw, or biw, or per day substantially continuously or continuously, in combination with a dosage of IP-10 containing an amount of from about 0.1 μg to about 50 μg, or from about 0.1 μg to about 50 ηg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α and IP-10 in the treatment of cancer in a patient comprising administering to the patient a dosage of INFERGEN® containing an amount of about 5 μg to about 45 μg of drug per dose of INFERGEN®, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α and IP-10 in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

Synergistic Combinations of a Type I Interferon Receptor Agonist and IP-10

In many embodiments, the effective amounts of a Type I interferon receptor agonist and IP-10 are synergistic amounts. As used herein, a “synergistic combination” or a “synergistic amount” of a Type I interferon receptor agonist and IP-10 is a combined dosage that is more effective in the therapeutic or prophylactic treatment of cancer than the incremental improvement in treatment outcome that could be predicted or expected from a merely additive combination of (i) the therapeutic or prophylactic benefit of the Type I interferon receptor agonist when administered at that same dosage as a monotherapy and (ii) the therapeutic or prophylactic benefit of IP-10 when administered at the same dosage as a monotherapy.

In some embodiments of the invention, a selected amount of a Type I interferon receptor agonist and a selected amount of IP-10 are effective when used in combination therapy for a disease, but the selected amount of Type I interferon receptor agonist and/or the selected amount of IP-10 is ineffective when used in monotherapy for the disease. Thus, the invention encompasses (1) regimens in which a selected amount of IP-10 enhances the therapeutic benefit of a selected amount of Type I interferon receptor agonist when used in combination therapy for a disease, where the selected amount of IP-10 provides no therapeutic benefit when used in monotherapy for the disease (2) regimens in which a selected amount of Type I interferon receptor agonist enhances the therapeutic benefit of a selected amount of IP-10 when used in combination therapy for a disease, where the selected amount of Type I interferon receptor agonist provides no therapeutic benefit when used in monotherapy for the disease and (3) regimens in which a selected amount of Type I interferon receptor agonist and a selected amount of IP-10 provide a therapeutic benefit when used in combination therapy for a disease, where each of the selected amounts of Type I interferon receptor agonist and IP-10, respectively, provides no therapeutic benefit when used in monotherapy for the disease. As used herein, a “synergistically effective amount” of Type I interferon receptor agonist and IP-10, and its grammatical equivalents, shall be understood to include any regimen encompassed by any of (1)-(3) above.

Type I Interferon Receptor Agonist and IP-10 in Combination Therapy with Pirfenidone or a Pirfenidone Analog

In one aspect, the present invention provides combination therapy for the treatment of cancer, comprising administering effective amounts of a Type I interferon receptor agonist, pirfenidone (or a pirfenidone analog), and IP-10. In some embodiments, the Type I interferon receptor agonist is IFN-α.

In general, effective dosages of pirfenidone or specific pirfenidone analogs can range from about 0.5 mg/kg/day to about 200 mg/kg/day, or at a fixed dosage of about 400 mg to about 3600 mg per day, or about 50 mg to about 10,000 mg per day, or about 100 mg to about 1,000 mg per day, or about 1,000 mg to about 3,000 mg per day, or about 1,000 mg to about 10,000 mg per day, administered orally, optionally in two or more divided doses per day. Other doses and formulations of pirfenidone and pirfenidone analogs suitable for use in a subject method for the treatment of cancer are described in U.S. Pat. Nos. 3,974,281; 3,839,346; 4,042,699; 4,052,509; 5,310,562; 5,518,729; 5,716,632; and 6,090,822.

Those of skill in the art will readily appreciate that dose levels of pirfenidone or pirfenidone analog can vary as a function of the specific compound, the severity of the symptoms and the susceptibility of the subject to side effects. Preferred dosages for a given compound are readily determinable by those of skill in the art by a variety of means.

Pirfenidone (or a pirfenidone analog) can be administered daily, twice a day, or three times a day, or in divided daily doses ranging from 2 to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In one embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 1 μg to about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 3 μg to about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN® consensus IFN-α, IP-10, and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of a consensus IFN-α, IP-10, and pirfenidone or a specific pirfenidone analog in the treatment of cancer in a patient comprising administering to the patient a dosage of PEGylated consensus IFN-α (PEG-CIFN) containing an amount of about 10 μg to about 150 μg of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of a consensus IFN-α, IP-10, and pirfenidone or a specific pirfenidone analog in the treatment of cancer in a patient comprising administering to the patient a dosage of monoPEG (30 kD, linear)-ylated consensus IFN-α containing an amount of about 5 μg to about 500 μg, or about or about 45 μg to about 450 μg, or about 60 μg to about 400 μg, or about 75 μg to about 350 μg, or abut 90 μg to about 300 μg, about 105 μg to about 270 μg, or about 120 μg to about 240 μg, or about 135 μg to about 210 μg, or about 150 μg to about 180 μg, or about 135 μg, of drug per dose, subcutaneously qw, qow, three times per month, or monthly, in combination with a dosage of pirfenidone or a specific pirfenidone analog containing an amount of about 50 mg to about 5,000 mg of drug orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 5 μg to about 150 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 5 μg to about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IP-10, and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

Type I Interferon Receptor Agonist and IP-10 in Combination Therapy with Type II Interferon Receptor Agonist

In another aspect, the present invention provides combination therapy for the treatment of cancer, comprising co-administering to the patient effective amounts of a Type I interferon receptor agonist, IP-10, and a Type II interferon receptor agonist. In some embodiments, the Type II interferon receptor agonist is IFN-γ.

Effective dosages of IFN-γ range from about 0.5 μg/m² to about 500 μg/m², usually from about 1.5 μg/m² to 200 μg/m², depending on the size of the patient. This activity is based on 10⁶ international units (U) per 50 μg of protein. IFN-γ can be administered daily, every other day, three times a week, twice per week, or substantially continuously or continuously for a period of from about 2 weeks to about 52 weeks, from about 52 weeks to about 2 years, or longer.

In certain embodiments, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m².

In one embodiment, the invention provides a method using an effective amount of INFERGEN®consensus IFN-α, IFN-γ, and IP-10 in the treatment of cancer in a patient comprising co-administering to the patient a dosage of INFERGEN® containing an amount of about 1 μg to about 30 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α, IFN-γ, and IP-10 in the treatment of cancer in a patient comprising co-administering to the patient a dosage of INFERGEN® containing an amount of about 3 μg to about 30 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α, IFN-γ, and IP-10 in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α, IFN-γ, and IP-10 in the treatment of cancer in a patient comprising co-administering to the patient a dosage of INFERGEN® containing an amount of about 30 μg of drug per dose of INFERGEN®, subcutaneously qd, qod, tiw, or biw, or per day substantially continuously or continuously, and a dosage of IFN-γ containing an amount of about 200 μg to about 300 μg of drug per dose of IFN-γ, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α, IFN-γ, and IP-10 in the treatment of cancer in a patient comprising co-administering to the patient a dosage of INFERGEN® containing an amount of about 5 μg to about 150 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously,.for the desired treatment duration.

In another embodiment, the invention provides a method using an effective amount of INFERGEN®consensus IFN-α, IFN-γ, and IP-10 in the treatment of cancer in a patient comprising co-administering to the patient a dosage of INFERGEN® containing an amount of about 5 μg to about 45 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment duration.

In another embodiment, the invention provides a method using an effective amount of INFERGEN®consensus IFN-α, IFN-γ, and IP-10 in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of IP-10 containing an amount of from about 0.1 μg to about 50 μg, or from about 0.1 μg to about 50 μg, of drug per dose of IP-10, intramuscularly qd, qod, tiw, or biw, or per day substantially continuously or continuously, for the desired treatment-duration.

Type I Interferon Receptor Agonist and IP-10 in Combination Therapy with Pirfenidone and Type II Interferon Receptor Agonist

In one aspect, the present invention provides combination therapy for the treatment of cancer, comprising administering effective amounts of IP-10, pirfenidone, a Type I interferon receptor agonist, and a Type II interferon receptor agonist. In some embodiments, the Type I interferon receptor agonist is IFN-α. In other embodiments, the Type II interferon receptor agonist is IFN-γ. In still other embodiments, the Type I interferon receptor is IFN-α and the Type II interferon receptor agonist is IFN-γ.

IFN-α and IFN-γ are typically administered subcutaneously. For example, IFN-α and IFN-γ can be administered subcutaneously qd, qod, tiw, or biw, or per day substantially continuously or continuously for a period of from about 2 weeks to about 52 weeks, from about 52 weeks to about 2 years, or longer.

In one embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 1 μg to about 30 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 50mg to about 5,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 3 μg to about 30 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 30 μg of drug per dose of INFERGEN®, subcutaneously qd, qod, tiw, or biw, or per day substantially continuously or continuously, and a dosage of IFN-γ containing an amount of about 200 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 5 μg to about 150 μg of drug per dose of INFERGEN®; subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising co-administering to the patient a dosage of INFERGEN® containing an amount of about 5 μg to about 45 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

In another embodiment, the invention provides a method using an effective amount of INFERGEN®consensus IFN-α, IFN-γ, IP-10, and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, and a dosage of IP-10 containing an amount of from about 0.1 μg to about 1000 μg of drug per dose of IP-10, intramuscularly or subcutaneously qd, qod, tiw, or biw, or per day substantially continuously or continuously, for the desired treatment duration.

Type I Interferon Receptor Agonist and IP-10 Combination Therapy as Adjuvant Therapy

In some embodiments, the present invention provides methods for combination therapy using a Type I interferon receptor agonist and IP-10, where the Type I interferon receptor agonist and IP-10 are administered as adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin,-rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e:g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc.. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (M7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use in connection with the methods of the invention include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ (8) colony-stimulating factors; (9) inhibitors of angiogenesis; and (10) antagonists of tumor necrosis factor.

In one aspect, the invention contemplates the combination of a Type I interferon receptor agonist and IP-10 as an adjuvant to any therapy in which the cancer patient receives treatment with least one additional antineoplastic drug, where the additional drug is a tyrosine kinase inhibitor. In some embodiments, the tyrosine kinase inhibitor is a receptor tyrosine kinase (RTK) inhibitor, such as type I receptor tyrosine kinase inhibitors (e.g., inhibitors of epidermal growth factor receptors), type II receptor tyrosine kinase inhibitors (e.g., inhibitors of insulin receptor), type III receptor tyrosine kinase inhibitors (e.g., inhibitors of platelet-derived growth factor receptor), and type IV receptor tyrosine kinase inhibitors (e.g., fibroblast growth factor receptor). In other embodiments, the tyrosine kinase inhibitor is a non-receptor tyrosine kinase inhibitor, such as inhibitors of src kinases or janus kinases.

In another aspect, the invention contemplates the combination of a Type I interferon receptor agonist and IP-10 as an adjuvant to any therapy in which the cancer patient receives treatment with least one additional antineoplastic drug, where the additional drug is an inhibitor of a receptor tyrosine kinase involved in growth factor signaling pathway(s). In some embodiments, the inhibitor is genistein. In other embodiments, the inhibitor is an EGFR tyrosine kinase-specific antagonist, such as IRESSA™ gefitinib (ZD18398; Novartis), TARCEVA™ erolotinib (OSI-774; Roche; Genentech; OSI Pharmaceuticals), or tyrphostin AG1478 (4-(3-chloroanilino)-6,7-diethoxyquinazoline. In still other embodiments, the inhibitor is any indolinone antagonist of Flk-1/KDR (VEGF-R2) tyrosine kinase activity described in U.S. Patent Application Publication No. 2002/0183364 A1, such as the indolinone antagonists of Flk-1/KDR (VEGF-R2) tyrosine kinase activity disclosed in Table 1 on pages 4-5 thereof. In further embodiments, the inhibitor is any of the substituted 3-[(4,5,6,7-tetrahydro-1H-indol-2-yl)methylene]-1,3-dihydroindol-2-one antagonists of Flk-1/KDR (VEGF-R2), FGF-R1 or PDGF-R tyrosine kinase activity disclosed in Sun, L., et al., J. Med. Chem., 43(14): 2655-2663 (2000). In additional embodiments, the inhibitor is any substituted 3-[(3- or 4-carboxyethylpyrrol-2-yl)methylidenyl]indolin-2-one antagonist of Flt-1 (VEGF-R1), Flk-1/KDR (VEGF-R2), FGF-R1 or PDGF-R tyrosine kinase activity disclosed in Sun, L., et al., J. Med. Chem. 42(25): 5120-5130 (1999).

In another aspect, the invention contemplates the combination of a Type I interferon receptor agonist and IP-10 as an adjuvant to any therapy in which the cancer patient receives treatment with least one additional antineoplastic drug, where the additional drug is an inhibitor of a non-receptor tyrosine kinase involved in growth factor signaling pathway(s). In some embodiments, the inhibitor is an antagonist of JAK2 tyrosine kinase activity, such as tyrphostin AG490 (2-cyano-3-(3,4-dihydroxyphenyl)-N-(benzyl)-2-propenamide). In other embodiments, the inhibitor is an antagonist of bcr-abl tyrosine kinase activity, such as GLEEVEC™ imatinib mesylate (STI-571; Novartis).

In another aspect, the invention contemplates the combination of a Type I interferon receptor agonist and IP-10 as an adjuvant to any therapy in which the cancer patient receives treatment with least one additional antineoplastic drug, where the additional drug is a serine/threonine kinase inhibitor. In some embodiments, the serine/threonine kinase inhibitor is a receptor serine/threonine kinase inhibitor, such as antagonists of TGF-β receptor serine/threonine kinase activity. In other embodiments, the serine/threonine kinase inhibitor is a non-receptor serine/threonine kinase inhibitor, such as antagonists of the serine/threonine kinase activity of the MAP kinases, protein kinase C (PKC), protein kinase A (PKA), or the cyclin-dependent kinases (CDKs).

In another aspect, the invention contemplates the combination of a Type I interferon receptor agonist and IP-10 as an adjuvant to any therapy in which the cancer patient receives treatment with least one additional antineoplastic drug, where the additional drug is an inhibitor of one or more kinases involved in cell cycle regulation In some embodiments, the inhibitor is an antagonist of CDK2 activation, such as tryphostin AG490 (2-cyano-3-(3,4-dihydroxyphenyl)-N-(benzyl)-2-propenamide). In other embodiments, the inhibitor is an antagonist of CDK1/cyclin B activity, such as alsterpaullone. In still other embodiments, the inhibitor is an antagonist of CDK2 kinase activity, such as indirubin-3′-monoxime. In additional embodiments, the inhibitor is an ATP pool antagonist, such as lometrexol (described in U.S. Patent Application Publication No. 2002/0156023 A1).

In another aspect, the invention contemplates the combination of a Type I interferon receptor agonist and IP-10 as an adjuvant to any therapy in which the cancer patient receives treatment with least one additional antineoplastic drug, where the additional drug is an a tumor-associated antigen antagonist, such as an antibody antagonist. In some embodiments involving the treatment of HER2-expressing tumors, the tumor-associated antigen antagonist is an anti-HER2 monoclonal antibody, such as HERCEPTIN™ trastuzumab. In some embodiments involving the treatment of CD20-expressing tumors, such as B-cell lymphomas, the tumor-associated antigen antagonist is an anti-CD20 monoclonal antibody, such as RITUXAN™ rituximab.

In another aspect, the invention contemplates the combination of a Type I interferon receptor agonist and IP-10 as an adjuvant to any therapy in which the cancer patient receives treatment with least one additional antineoplastic drug, where the additional drug is a tumor growth factor antagonist. In some embodiments, the tumor growth factor antagonist is an antagonist of epidermal growth factor (EGF), such as an anti-EGF monoclonal antibody. In other embodiments, the tumor growth factor antagonist is an antagonist of epidermal growth factor receptor erbB1 (EGFR), such as an anti-EGFR monoclonal antibody inhibitor of EGFR activation or signal transduction.

In another aspect, the invention contemplates the combination of a Type I interferon receptor agonist and IP-10 as an adjuvant to any therapy in which the cancer patient receives treatment with least one additional antineoplastic drug, where the additional drug is an Apo-2 ligand agonist. In some embodiments, the Apo-2 ligand agonist is any of the Apo-2 ligand polypeptides described in WO 97/25428.

In another aspect, the invention contemplates the combination of a Type I interferon receptor agonist and IP-10 as an adjuvant to any therapy in which the cancer patient receives treatment with least one additional antineoplastic drug, where the additional drug is an anti-angiogenic agent. In some embodiments, the anti-angiogenic agent is a vascular endothelial cell growth factor (VEGF) antagonist, such as an anti-VEGF monoclonal antibody, e.g. AVASTIN™ bevacizumab (Genentech). In other embodiments, the anti-angiogenic agent is a retinoic acid receptor (RXR) ligand, such as any RXR ligand described in U.S. Patent Application Publication No. 2001/0036955 A1 or in any of U.S. Pat. Nos. 5,824,685; 5,780,676; 5,399,586; 5,466,861; 4,810,804; 5,770,378; 5,770,383; or 5,770,382. In still other embodiments, the anti-angiogenic agent is a peroxisome proliferator-activated receptor (PPAR) gamma ligand, such as any PPAR gamma ligand described in U.S. Patent Application Publication No. 2001/0036955 A1.

Determining Susceptibility of Tumor to Type I Interferon Receptor Agonist and IP-10 Combination Therapy

The present invention further provides methods for determining the susceptibility or sensitivity of a tumor to growth inhibition by Type I interferon receptor agonist and IP-10 combination therapy. The methods generally involve culturing a patient's tumor cell in vitro in a medium comprising a Type I interferon receptor agonist and IP-10; and determining the effect, if any, of the Type I interferon receptor agonist and IP-10 on the survival of the cell. A reduction in the survival of the cell, compared with the survival in the absence of Type I interferon receptor agonist and IP-10, indicates that the tumor is susceptible to treatment with Type I interferon receptor agonist and IP-10 combination therapy.

For example, a reduction of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, in the cell survival when cultured in the presence of a Type I interferon receptor agonist and IP-10, compared with the cell survival in the absence of the Type I interferon receptor agonist and IP-10, indicates that the tumor is susceptible to treatment with the Type I interferon receptor agonist and IP-10 combination therapy.

Sensitivity of a tumor cell to Type I interferon receptor agonist plus IP-10 treatment is determined using any known method. Typically, a biopsy sample is obtained using standard procedures, and cell from the biopsied tissue are cultured in vitro. The method generally involves culturing cells from the biopsied tissue in vitro in the presence of Type I interferon receptor agonist and IP-10, and, after a suitable time, determining the number of live cells in the culture, compared to the number of live cells in a culture not treated with Type I interferon receptor agonist and IP-10. Live cells can be distinguished from dead cells using any standard assay method, including, but not limited to, a trypan blue dye exclusion assay; an MTT (3-(4,5-dimethylthiazol-2-yl)-2,5-diphenyl-2 H-tetrazolium bromide) assay; a flow cytometric assay that relies upon exclusion of a dye from live, but not dead cells, e.g., propidium iodide uptake (where propidium iodide is taken up by dead, but not live cells), uptake of a Hoechst dye, such as Hoechst 33342, that enters live, but not dead cells, and the like, which assays are used in conjunction with fluorescence activated cell sorter to distinguish live from dead cells. For example, survival is determined using a method as described in the Example.

In some embodiments, the invention provides methods of treating cancer in an individual having a cancer susceptible to treatment with Type I interferon receptor agonist and IP-10, the method comprising determining the susceptibility of the cancer to treatment with Type I interferon receptor agonist and IP-10; and administering an effective amount of Type I interferon receptor agonist and IP-10 to the individual.

Pirfenidone and an Additional Cancer Therapeutic Agent in Combination Therapy

In some embodiments, pirfenidone or a pirfenidone analog is administered during the entire course of treatment with an additional antineoplastic agent or biological response modifier. In other embodiments, pirfenidone or a pirfenidone analog is administered for a period of time that is overlapping with the course of treatment with the additional antineoplastic agent/biological response modifier, e.g., the pirfenidone or pirfenidone analog treatment can begin before the treatment with the additional agent begins and end before treatment with the additional agent ends; the pirfenidone or pirfenidone analog treatment can begin after the treatment with the additional agent begins and end after the treatment with the additional agent ends; the pirfenidone or pirfenidone analog treatment can begin after the treatment with the additional agent begins and end before the treatment with the additional agent ends; or the pirfenidone or pirfenidone analog treatment can begin before the treatment with the additional agent begins and end after the treatment with the additional agent ends.

In many embodiments, pirfenidone or pirfenidone analog and an additional antineoplastic agent or biological response modifier are administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. In embodiments in which the additional antineoplastic agent or biological response modifier is an interferon receptor agonist, the interferon receptor agonist can be administered tid, bid, qd, qod, biw, tiw, qw, qow, three times per month, once monthly, substantially continuously, or continuously.

Those of skill will readily appreciate that dose levels can vary as a function of the specific agent used (e.g., the particular antineoplastic agent or biological response modifier), the severity of the symptoms and the susceptibility of the subject to side effects. Preferred dosages for a given agent are readily determinable by those of skill in the art by a variety of means.

In general, effective dosages of pirfenidone or specific pirfenidone analogs can range from about 0.5 mg/kg/day to about 200 mg/kg/day, or at a fixed dosage of about 400 mg to about 3600 mg per day, or about 50 mg to about 10,000 mg per day, or about 100 mg to about 1,000 mg per day, or about 1,000 mg to about 3,000 mg per day, or about 1,000 mg to about 10,000 mg per day, administered orally, optionally in two or more divided doses per day. Other doses and formulations of pirfenidone and pirfenidone analogs suitable for use in a subject method for the treatment of cancer are described in U.S. Pat. Nos. 3,974,281; 3,839,346; 4,042,699; 4,052,509; 5,310,562; 5,518,729; 5,716,632; and 6,090,822.

Those of skill in the art will readily appreciate that dose levels of pirfenidone or pirfenidone analog can vary as a function of the specific compound, the severity of the symptoms and the susceptibility of the subject to side effects. Preferred dosages for a given compound are readily determinable by those of skill in the art by a variety of means.

Pirfenidone (or a pirfenidone analog) can be administered daily, twice a day, or three times a day, or in divided daily doses ranging from 2 to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Pirfenidone (or pirfenidone analog) and an additional antineoplastic agent or biological response modifier are generally administered in separate formulations. Pirfenidone (or pirfenidone analog) and the additional agent may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

Pirfenidone in Combination Therapy with Type I Interferon Receptor Agonist

In one aspect, the present invention provides combination therapy for the treatment of cancer, comprising administering effective amounts of pirfenidone and a Type I interferon receptor agonist. In some embodiments, the Type I interferon receptor agonist is IFN-α.

Effective dosages of IFN-α can range from 0.3 μg to 100 μg. Effective dosages of Infergen® consensus IFN-α can contain an amount of about 3 μg, about 9 μg, about 15 μg, about 18 μg, or about 27 μg, or about 30 μg of drug per dose. Effective dosages of IFN-α2a and IFN-α2b can contain an amount of about 3 million Units (MU) to about 30 MU of drug per dose. Effective dosages of PEGASYS®PEGylated IFN-α2a can contain an amount of about 5 μg to about 500 μg, or about 45 μg to about 450 μg, or about 60 μg to about 400 μg, or about 75 μg to about 350 μg, or about 90 μg to about 300 μg, about 105 μg to about 270 μg, or about 120 μg to about 240 μg, or about 135 μg to about 210 μg, or about 150 μg to about 180 μg, or about 135 μg, of drug per dose. Effective dosages of PEG-INTRON®PEGylated IFN-α2b can contain an amount of about 0.5 μg to about 5.0 μg, or about 0.75 μg to about 3.5 μg, or about 1.0 μg to about 3.0 μg, or about 1.25 μg to about 2.5 μg, or about 1.5 μg to about 2.0 μg, of drug per kg of body weight per dose. Effective dosages of PEGylated consensus interferon (PEG-CIFN) can contain an amount of about 9 μg to about 200 μg, or about 12 μg to about 180 μg, or about 15 μg to about 150 μg, or about 18 μg to about 120 μg, or about 21 μg to about 90 μg, or about 24 μg to about 75 μg, or about 27 μg to about 60 μg, or about 45 μg, of CIFN amino acid weight per dose of PEG-CIFN. Effective dosages of monoPEG (30 kD, linear)-ylated CIFN can contain an amount of about 5 μg to about 500 μg, or about or about 45 μg to about 450 μg, or about 60 μg to about 400 μg, or about 75 μg to about 350 μg, or about 90 μg to about 300 μg, about 105 μg to about 270 μg, or about 120 μg to about 240 μg, or about 135 μg to about 210 μg, or about 150 μg to about 180 μg, or about 135 μg, of drug per dose.

IFN-α is typically administered subcutaneously. For example, IFN-α can be administered subcutaneously qd, qod, tiw, or biw, or per day substantially continuously or continuously for a period of from about 2 weeks to about 52 weeks, from about 52 weeks to about 2 years, or longer.

In one embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 1 μg to about 30 μg of drug per dose of INFERGEN® subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 3 μg to about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, for the desired treatment duration.

In another embodiment, the invention-provides a method using an effective amount of INFERGEN®consensus IFN-α and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 30 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, for the desired treatment duration.

In another embodiment, the invention provides a method using an effective amount of a consensus IFN-α and pirfenidone or a specific pirfenidone analog in the treatment of cancer in a patient comprising administering to the patient a dosage of PEGylated consensus IFN-α (PEG-CIFN) containing an amount of about 10 μg to about 150 μg of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose orally qd, optionally in two or more divided doses per day, for the desired treatment duration.

In another embodiment, the invention provides a method using an effective amount of a consensus IFN-α and pirfenidone or a specific pirfenidone analog in the treatment of cancer in a patient comprising administering to the patient a dosage of monoPEG (30 kD, linear)-ylated consensus IFN-α containing an amount of about 5 μg to about 500 μg, or about or about 45 μg to about 450 μg; or about 60 μg to about 400 μg, or about 75 μg to about 350 μg, or abut 90 μg to about 300 μg, about 105 μg to about 270 μg, or about 120 μg to about 240 μg, or about 135 μg to about 210 μg, or about 150 μg to about 180 μg, or about 135 μg, of drug per dose, subcutaneously qw, qow, three times per month, or monthly, in combination with a dosage of pirfenidone or a specific pirfenidone analog containing an amount of about 50 mg to about 5,000 mg of drug orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 5 μg to about 150 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 5 μg to about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, for the desired treatment duration.

Pirfenidone in Combination Therapy with Type II Interferon Receptor Agonist

In another aspect, the present invention provides combination therapy for the treatment of cancer, comprising co-administering to the patient effective amounts of pirfenidone and a Type II interferon receptor agonist In some embodiments, the Type II interferon receptor agonist is IFN-γ.

Effective dosages of IFN-γ range from about 0.5 μg/m² to about 500 μg/m², usually from about 1.5 μg/m² to 200 μg/m², depending on the size of the patient. This activity is based on 10⁶ international units (U) per 50 μg of protein IFN-γ can be administered daily, every other day, three times a week, twice per week, or substantially continuously or continuously for a period of from about 2 weeks to about 52 weeks, from about 52 weeks to about 2 years, or longer.

In certain embodiments, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-65 per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m².

In one embodiment, the invention provides a method using an effective amount of IFN-γ and pirfenidone or a specific pirfenidone analog in the treatment of cancer in a patient comprising administering to the patient a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, for the desired treatment duration.

In another embodiment, the invention provides a method using an effective amount of IFN-γ and pirfenidone or a specific pirfenidone analog in the treatment of cancer in a patient comprising administering to the patient a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 100 mg to about 1,000 mg of drug per dose orally qd, optionally in two or more divided doses per day, for the desired-treatment duration.

In another embodiment, the invention provides a method using an effective amount of IFN-γ and pirfenidone or a specific pirfenidone analog in the treatment of cancer in a patient comprising administering to the patient a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, for the desired treatment duration.

In one embodiment, the invention provides a method using an effective amount of IFN-γ and pirfenidone or a specific pirfenidone analog in the treatment of cancer in a patient comprising administering to the patient a dosage of IFN-γ containing an amount of about 200 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 5,000 mg of drug per dose orally qd, optionally in two or more divided doses per day, for the desired treatment duration.

Pirfenidone in Combination Therapy with Type I Interferon Receptor Agonist and Type II Interferon Receptor Agonist

In one aspect, the present invention provides combination therapy for the treatment of cancer, comprising administering effective amounts of pirfenidone or a pirfenidone analog, a Type I interferon receptor agonist, and a Type II interferon receptor agonist. In some embodiments, the Type I interferon receptor agonist is IFN-α. In other embodiments, the Type II interferon receptor agonist is IFN-γ. In still other embodiments, the Type I interferon receptor is IFN-α and the Type II interferon receptor agonist is IFN-γ.

IFN-α and IFN-γ are typically administered subcutaneously. For example, IFN-α and IFN-γ can be administered subcutaneously qd, qod, tiw, or biw, or per day substantially continuously or continuously for a period of from about 2 weeks to about 52 weeks, from about 52 weeks to about 2 years, or longer.

In one embodiment, the invention provides a method using an effective amount of INFERGEN®consensus IFN-α, IFN-γ, and pirfenidone or a specific pirfenidone analog in the treatment of cancer in a patient comprising co-administering to the patient a dosage of INFERGEN® containing an amount of about 1 μg to about 30 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 50 mg to about 5,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising co-administering to the patient a dosage of INFERGEN® containing an amount of about 3 μg to about 30 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 300 mg to about 3,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising administering to the patient a dosage of INFERGEN® containing an amount of about 10 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 500 mg of drug per dose orally qd, optionally in two or more divided doses per day, for the desired treatment duration.

In another embodiment, the invention provides a method using an effective amount of INFERGEN®consensus IFN-α, IFN-γ and pirfenidone or a specific pirfenidone analog in the treatment of cancer in a patient comprising co-administering to the patient a dosage of INFERGEN® containing an amount of about 30 μg of drug per dose of INFERGEN®, subcutaneously qd, qod, tiw, or biw, or per day substantially continuously or continuously, and a dosage of IFN-γ containing an amount of about 200 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising co-administering to the patient a dosage of INFERGEN® containing an amount of about 5 μg to about 150 μg of drug per dose of INFERGEN®, subcutaneously qd, qod, tiw, or biw, or per day substantially continuously or continuously, and a dosage of IFN-γ containing an amount of about 10 μg to about 300 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising co-administering to the patient a dosage of INFERGEN® containing an amount of about 5 μg to about 45 μg of drug per dose of INFERGEN®, subcutaneously qd, qod, tiw, or biw, or per day substantially continuously or continuously, and a dosage of IFN-γ containing an amount of about 10 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose orally qd, optionally in two or more divided doses per day, for the desired treatment duration.

In another embodiment, the invention provides a method using an effective amount of INFERGEN®consensus IFN-α, IFN-γ, and pirfenidone or a specific pirfenidone analog in the treatment of cancer in a patient comprising administering to the patient a dosage of INFERGEN® containing an amount of about 45 μg of drug per dose of INFERGEN®, subcutaneously qd, qod, tiw, or biw, or per day substantially continuously or continuously, and a dosage of IFN-γ containing an amount of about 90 μg to about 100 μg of drug per dose of IFN-γ, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose orally qd, optionally in two or more divided doses per day, for the desired treatment duration.

Pirfenidone Combination Therapy as Adjuvant Therapy

In some embodiments, the present invention provides methods for combination therapy using pirfenidone, where the pirfenidone is administered as adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e;g mitoxantrone, azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc.. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chlorofluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (M7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use in connection with the methods of the invention include, but-are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ (8) colony-stimulating factors; (9) inhibitors of angiogenesis; and (10) antagonists of tumor necrosis factor.

In one aspect, the invention contemplates pirfenidone or pirfenidone analog as an adjuvant to any therapy in which the cancer patient receives treatment with least one additional antineoplastic drug, where the additional drug is a tyrosine kinase inhibitor. In some embodiments, the tyrosine kinase inhibitor is a receptor tyrosine kinase (RTK) inhibitor, such as type I receptor tyrosine kinase inhibitors (e.g., inhibitors of epidermal growth factor receptors), type II receptor tyrosine kinase inhibitors (e.g., inhibitors of insulin receptor), type III receptor tyrosine kinase inhibitors (e.g., inhibitors of platelet-derived growth factor receptor), and type IV receptor tyrosine kinase inhibitors (e.g., fibroblast growth factor receptor). In other embodiments, the tyrosine kinase inhibitor is a non-receptor tyrosine kinase inhibitor, such as inhibitors of src kinases or janus kinases.

In another aspect, the invention contemplates pirfenidone or pirfenidone analog as an adjuvant to any therapy in which the cancer patient receives treatment with least one additional antineoplastic drug, where the additional drug is an inhibitor of a receptor tyrosine kinase involved in growth factor signaling pathway(s). In some embodiments, the inhibitor is genistein. In other embodiments, the inhibitor is an EGFR tyrosine kinase-specific antagonist, such as IRESSA™ gefitinib (ZD18398; Novartis), TARCEVA™ erolotinib (OSI-774; Roche; Genentech; OSI Pharmaceuticals), or tyrphostin AG1478 (4-(3-chloroanilino)-6,7-dimethoxyquinazoline. In still other embodiments, the inhibitor is any indolinone antagonist of Flk-1/KDR (VEGF-R2) tyrosine kinase activity described in U.S. Patent Application Publication No. 2002/0183364 A1, such as the indolinone antagonists of Flk-1/KDR (VEGF-R2) tyrosine kinase activity disclosed in Table 1 on pages 4-5 thereof. In further embodiments, the inhibitor is any of the substituted 3-[(4,5,6,7-tetrahydro-1H-indol-2-yl)methylene]-1,3-dihydroindol-2-one antagonists of Flk-1/KDR (VEGF-R2), FGF-R1 or PDGF-R tyrosine kinase activity disclosed in Sun, L., et al., J. Med. Chem., 43(14): 2655-2663 (2000). In additional embodiments, the inhibitor is any substituted 3-[(3- or 4-carboxyethylpyrrol-2-yl)methylidenyl]indolin-2-one antagonist of Flt-1 (VEGF-R1), Flk-1/KDR (VEGF-R2), FGF-R1 or PDGF-R tyrosine kinase activity disclosed in Sun, L., et al., J. Med. Chem., 42(25): 5120-5130 (1999).

In another aspect, the invention contemplates pirfenidone or pirfenidone analog as an adjuvant to any therapy in which the cancer patient receives treatment with least one additional antineoplastic drug, where the additional drug is an inhibitor of a non-receptor tyrosine kinase involved in growth factor signaling pathway(s). In some embodiments, the inhibitor is an antagonist of JAK2 tyrosine kinase activity, such as tyrphostin AG490 (2-cyano-3-(3,4-dihydroxyphenyl)-N-(benzyl)-2-propenamide). In other embodiments, the inhibitor is an antagonist of bcr-abl tyrosine kinase activity, such as GLEEVEC™ imatinib mesylate (STI-571; Novartis).

In another aspect, the invention contemplates pirfenidone or pirfenidone analog as an adjuvant to any therapy in which the cancer patient receives treatment with least one additional antineoplastic drug, where the additional drug is a serine/threonine kinase inhibitor. In some embodiments, the serine/threonine kinase inhibitor is a receptor serine/threonine kinase inhibitor, such as antagonists of TGF-β receptor serine/threonine kinase activity. In other embodiments, the serine/threonine kinase inhibitor is a non-receptor serine/threonine kinase inhibitor, such as antagonists of the serine/threonine kinase activity of the MAP kinases, protein kinase C (PKC), protein kinase A (PKA), or the cyclin-dependent kinases (CDKs).

In another aspect, the invention contemplates pirfenidone or pirfenidone analog as an adjuvant to any therapy in which the cancer patient receives treatment with least one additional antineoplastic drug, where the additional drug is an inhibitor of one or more kinases involved in cell cycle regulation. In some embodiments, the inhibitor is an antagonist of CDK2 activation, such as tryphostin AG490 (2-cyano-3-(3,4-dihydroxyphenyl)-N-(benzyl)-2-propenamide). In other embodiments, the inhibitor is an antagonist of CDK1/cyclin B activity, such as alsterpaullone. In still other embodiments, the inhibitor is an antagonist of CDK2 kinase activity, such as indirubin-3′-monoxime. In additional embodiments, the inhibitor is an ATP pool antagonist, such as lometrexol (described in U.S. Patent Application Publication No. 2002/0156023 A1).

In another aspect, the invention contemplates pirfenidone or pirfenidone analog as an adjuvant to any therapy in which the cancer patient receives treatment with least one additional antineoplastic drug, where the additional drug is an a tumor-associated antigen antagonist, such as an antibody antagonist. In some embodiments involving the treatment of HER2-expressing tumors, the tumor-associated antigen antagonist is an anti-HER2 monoclonal antibody, such as HERCEPTIN™ trastuzumab. In some embodiments involving the treatment of CD20-expressing tumors, such as B-cell lymphomas, the tumor-associated antigen antagonist is an anti-CD20 monoclonal antibody, such as RITUXAN™ rituximab.

In another aspect, the invention contemplates pirfenidone or pirfenidone analog as an adjuvant to any therapy in which the cancer patient receives treatment with least one additional antineoplastic drug, where the additional drug is a tumor growth factor antagonist. In some embodiments, the tumor growth factor antagonist is an antagonist of epidermal growth factor (EGF), such as an anti-EGF monoclonal antibody. In other embodiments, the tumor growth factor antagonist is an antagonist of epidermal growth factor receptor erbB1 (EGFR), such as an anti-EGFR monoclonal antibody inhibitor of EGFR activation or signal transduction.

In another aspect, the invention contemplates pirfenidone or pirfenidone analog as an adjuvant to any therapy in which the cancer patient receives treatment with least one additional antineoplastic drug, where the additional drug is an Apo-2 ligand agonist. In some embodiments, the Apo-2 ligand agonist is any of the Apo-2 ligand polypeptides described in WO 97/25428.

In another aspect, the invention contemplates pirfenidone or pirfenidone analog as an adjuvant to any therapy in which the cancer patient receives treatment with least one additional antineoplastic drug, where the additional drug is an anti-angiogenic agent. In some embodiments, the anti-angiogenic agent is a vascular endothelial cell growth factor (VEGF) antagonist, such as an anti-VEGF monoclonal antibody, e.g. AVASTIN™ bevacizumab (Genentech). In other embodiments, the anti-angiogenic agent is an antagonist of VEGF-R1, such as an anti-VEGF-R1 monoclonal antibody. In other embodiments, the anti-angiogenic agent is an antagonist of VEGF-R2, such as an anti-VEGF-R2 monoclonal antibody. In other embodiments, the anti-angiogenic agent is an antagonist of basic fibroblast growth factor (bFGF), such as an anti-bFGF monoclonal antibody. In other embodiments, the anti-angiogenic agent is an antagonist of bFGF receptor, such as an anti-bFGF receptor monoclonal antibody. In other embodiments, the anti-angiogenic agent is an antagonist of TGF-β, such as an anti-TGF-β monoclonal antibody. In other embodiments, the anti-angiogenic agent is an antagonist of TGF-β receptor, such as an anti-TGF-β receptor monoclonal antibody. In other embodiments, the anti-angiogenic agent is a retinoic acid receptor (RXR) ligand, such as any RXR ligand described in U.S. Patent Application Publication No. 2001/0036955 A1 or in any of U.S. Pat. Nos. 5,824,685; 5,780,676; 5,399,586; 5,466,861; 4,810,804; 5,770,378; 5,770,383; or 5,770,382. In still other embodiments, the anti-angiogenic agent is a peroxisome proliferator-activated receptor (PPAR) gamma ligand, such as any PPAR gamma ligand described in U.S. Patent Application Publication No. 2001/0036955 A1.

Exemplary non-limiting examples of combination therapies that include treatment with radiation and pirfenidone, or treatment with an additional chemotherapeutic agent and pirfenidone, are as follows:

1) a dosage of pirfenidone or a specific pirfenidone analog containing an amount of from about 100 mg to about 1000 mg of drug per dose; and cisplatin in a dosage range of from about 5 mg/m² to about 150 mg/m²;

2) a dosage of pirfenidone or a specific pirfenidone analog containing an amount of from about 100 mg to about 1000 mg of drug per dose; and carboplatin in a dosage range of from about 5 mg/m² to about 1000 mg/m²;

3) a dosage of pirfenidone or a specific pirfenidone analog containing an amount of from about 100 mg to about 1000 mg of drug per dose; and radiation in a dosage range of from about 200 cGy to about 8000 cGy;

4) a dosage of pirfenidone or a specific pirfenidone analog containing an amount of from about 100 mg to about 1000 mg of drug per dose; and paclitaxel in a dosage range of from about 40 mg/m² to about 250 mg/m²;

5) a dosage of pirfenidone or a specific pirfenidone analog containing an amount of from about 100 mg to about 1000 mg of drug per dose; paclitaxel in a dosage range of from about 40 mg/m² to about 250 mg/m²; and carboplatin in a dosage range of from about 5 mg/m² to about 1000 mg/m²;

6) a dosage of pirfenidone or a specific pirfenidone analog containing an amount of from about 100 mg to about 1000 mg of drug per dose; 5FU in a dosage range of from about 5 mg/m² to about 5000 mg/m²; and leucovorin in a dosage range of from about 5 mg/m² to about 1000 mg/m²;

7) a dosage of pirfenidone or a specific pirfenidone analog containing an amount of from about 100 mg to about 1000 mg of drug per dose; and trastuzumab in an initial loading dose of 4 mg/kg and a weekly maintenance dose of 2 mg/kg;

8) a dosage of pirfenidone or a specific pirfenidone analog containing an amount of from about 100 mg to about 1000 mg of drug per dose; trastuzumab in an initial loading dose of 4 mg/kg and a weekly maintenance dose of 2 mg/kg; and paclitaxel in a dosage range of from about 40 mg/m² to about 250 mg/m²;

9) a dosage of pirfenidone or a specific pirfenidone analog containing an amount of from about 100 mg to about 1000 mg of drug per dose; paclitaxel in a dosage range of from about 40 mg/m² to about 250 mg/m²; and estramustine phosphate (Emcyte®) in a dosage range of from about 5 mg/m² to about 1000 mg/m²;

10) a dosage of pirfenidone or a specific pirfenidone analog containing an amount of from about 100 mg to about 1000 mg of drug per dose; cisplatin in a dosage range of from about 5 mg/m² to about 150 mg/m²; and 5FU in a dosage range of from about 5 mg/m² to about 5000 mg/m²;

11) a dosage of pirfenidone or a specific pirfenidone analog containing an amount of from about 100 mg to about 1000 mg of drug per dose; 5FU in a dosage range of from about 5 mg/m² to about 5000 mg/m²; and radiation in a dose of from about 200 cGy to about 8000 cGy;

12) a dosage of pirfenidone or a specific pirfenidone analog containing an amount of from about 100 mg to about 1000 mg of drug per dose; 5FU in a dosage range of from about 5 mg/m² to about 5000 mg/m²; and paclitaxel in a dosage range of from about 40 mg/m² to about 250 mg/m².

Determining Susceptibility of Tumor to Combination Therapy with Pirfenidone and an Additional Agent

The present invention further provides methods for determining the susceptibility or sensitivity of a tumor to growth inhibition by pirfenidone in combination with an additional antineoplastic agent or biological response modifier. The methods generally involve culturing a patient's tumor cell in vitro in a medium comprising pirfenidone and an additional agent; and determining the effect, if any, of pirfenidone and the additional agent on the survival of the cell. A reduction in the survival of the cell, compared with the survival in the absence of pirfenidone and the additional agent, indicates that the tumor is susceptible to treatment with a combination of pirfenidone and the additional agent.

For example, a reduction of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, in the cell survival when cultured in the presence of pirfenidone and the additional agent, compared with the cell survival in the absence of pirfenidone and the additional agent, indicates that the tumor is susceptible to treatment with a combination of pirfenidone and the additional agent.

Sensitivity of a tumor cell to treatment with pirfenidone plus an additional agent is determined using any known method. Typically, a biopsy sample is obtained using standard procedures, and cell from the biopsied tissue are cultured in vitro. The method generally involves culturing cells from the biopsied tissue in vitro in the presence of pirfenidone and the additional agent, and, after a suitable time, determining the number of live cells in the culture, compared to the number of live cells in a culture not treated with pirfenidone and the additional agent. Live cells can be distinguished from dead cells using any standard assay method, including, but not limited to, a trypan blue dye exclusion assay; an MTT (3-(4,5-dimethylthiazol-2-yl)-2,5-diphenyl-2H-tetrazolium bromide) assay; a flow cytometric assay that relies upon exclusion of a dye from live, but not dead cells, e.g., propidium iodide uptake (where propidium iodide is taken up by dead, but not live cells), uptake of a Hoechst dye, such as Hoechst 33342, that enters live, but not dead cells, and the like, which assays are used in conjunction with fluorescence activated cell sorter to distinguish live from dead cells.

In some embodiments, the invention provides methods of treating cancer in an individual having a cancer susceptible to treatment with pirfenidone and an additional agent, the method comprising determining the susceptibility of the cancer to treatment with pirfenidone and the additional agent; and administering an effective amount of pirfenidone and the additional agent to the individual.

Determining Efficacy of Treatment

Whether a tumor load has been decreased can be determined using any known method, including, but not limited to, measuring solid tumor mass; counting the number of tumor cells using cytological assays; fluorescence-activated cell sorting (e.g., using antibody specific for a tumor-associated antigen) to determine the number of cells bearing a given tumor antigen; computed tomography scanning, magnetic resonance imaging, and/or x-ray imaging of the tumor to estimate and/or monitor tumor size; measuring the amount of tumor-associated antigen in a biological sample, e.g., blood; and the like.

Whether growth of a tumor is inhibited can be determined using any known method, including, but not limited to, a proliferation assay as described in the Example; a ³H-thymidine uptake assay; and the like.

Subjects Suitable for Treatment

Subjects suitable for treatment with a method of the present invention include individuals having any type of cancer. Of particular interest in many embodiments is the treatment of humans. In some embodiments, a subject suitable for treatment with a subject method is an individual having a cancer, who was previously treated with a cancer therapy for the cancer, but who failed to respond to the previous cancer therapy, or in whom the cancer initially responded to the previous cancer therapy, but who experienced a recurrence of the cancer.

In particular embodiments, a suitable subject is one having a cancer that is susceptible to treatment with IP-10 and pirfenidone combination therapy.

In particular embodiments, a suitable subject is one having a cancer that is susceptible to treatment with Type I interferon receptor agonist and pirfenidone combination therapy. In many embodiments, treatment of human subjects is of interest.

In particular embodiments, a suitable subject is one having a cancer that is susceptible to treatment with IP-10 and Type I interferon receptor agonist combination therapy. In many embodiments, treatment of human subjects is of interest.

In particular embodiments, a suitable subject is one having a cancer that is susceptible to treatment with a combination of pirfenidone and an additional antineoplastic agent or biological response modifier.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s, second(s); min, minute(s); hr, hour(s); and the like.

### Example 1

### Susceptibility of Cancer Cells to Growth Inhibition by IP-10 and Pirfenidone

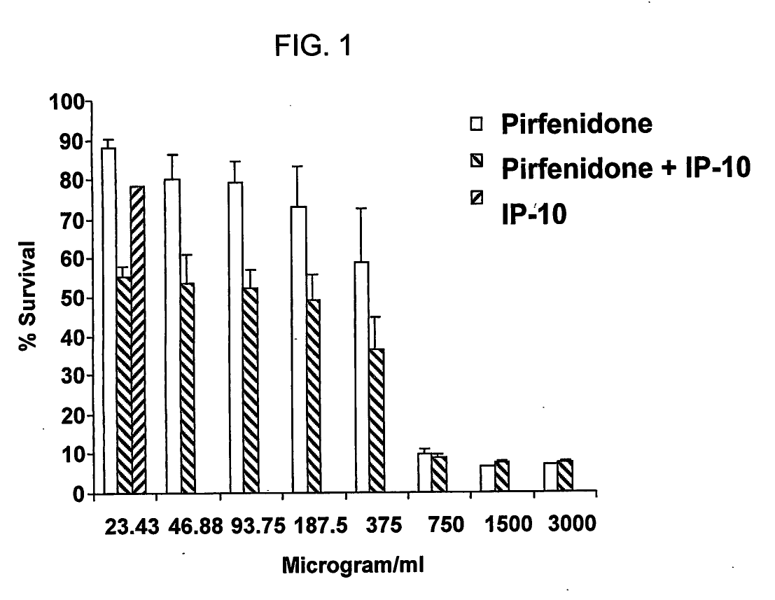

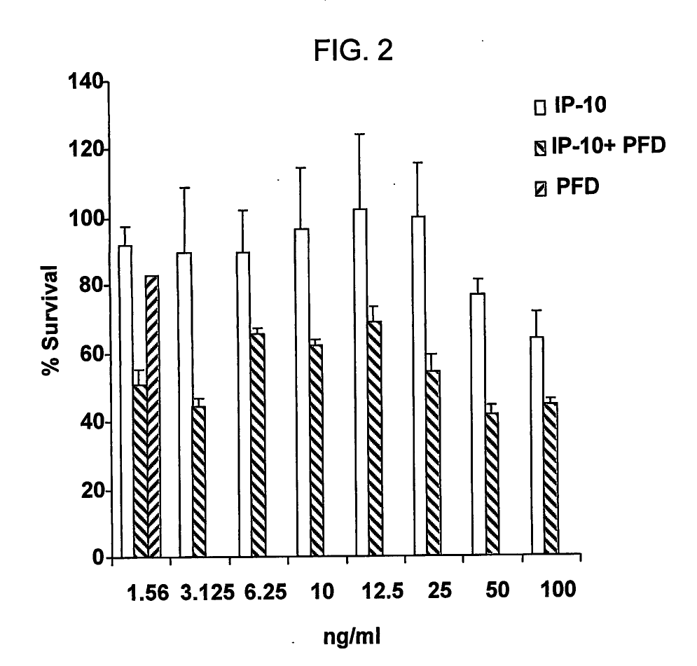

CAOV-3 cells (American Type Culture Collection No. HTB-75) were grown in the presence of IP-10, pirfenidone, or a combination of IP-10 and pirfenidone. Control cell cultures were grown without IP-10 or pirfenidone. The results are shown in FIGS. 1 and 2.

FIG. 1 depicts the antiproliferative effects of various amounts of pirfenidone alone or in combination with 10 ng/ml IP-10 on the CAOV-3 cell line.

FIG. 2 depicts the antiproliferative effects of various amounts of IP-10 in combination with 30 μg/ml pirfenidone on the CAOV-3 cell line.

### Example 2

### Susceptibility of Cancer Cells to Growth Inhibition by INFERGEN® and Pirfenidone

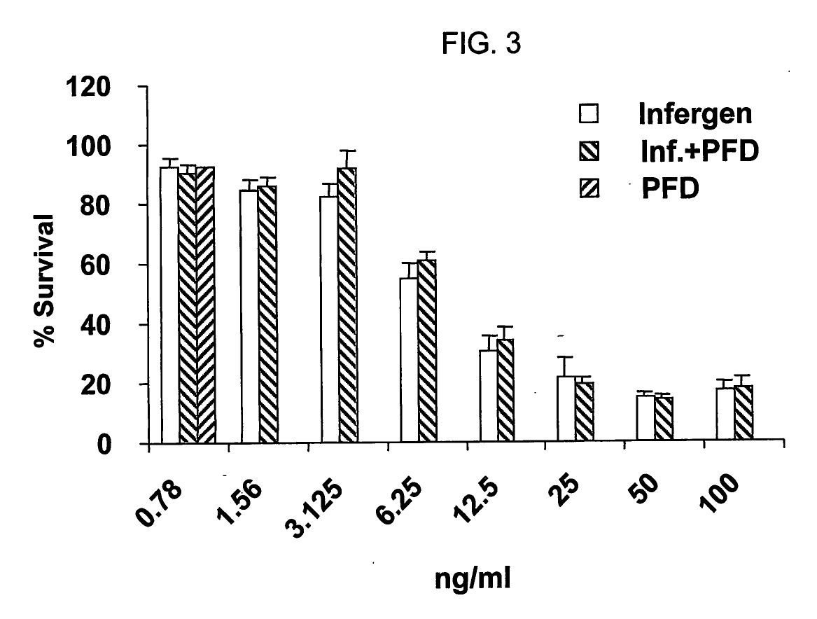

CAOV-3 cells (American Type Culture Collection No. HTB-75) or OVCAR were grown in the presence of INFERGEN®, pirfenidone, or a combination of INFERGEN® and pirfenidone. Control cell cultures were grown without INFERGEN® or pirfenidone. The results are shown in FIGS. 3-5.

FIG. 3 depicts the antiproliferative effects of various amounts of INFERGEN® alone or in combination with 30 μg/ml pirfenidone on proliferation of OVCAR cells.

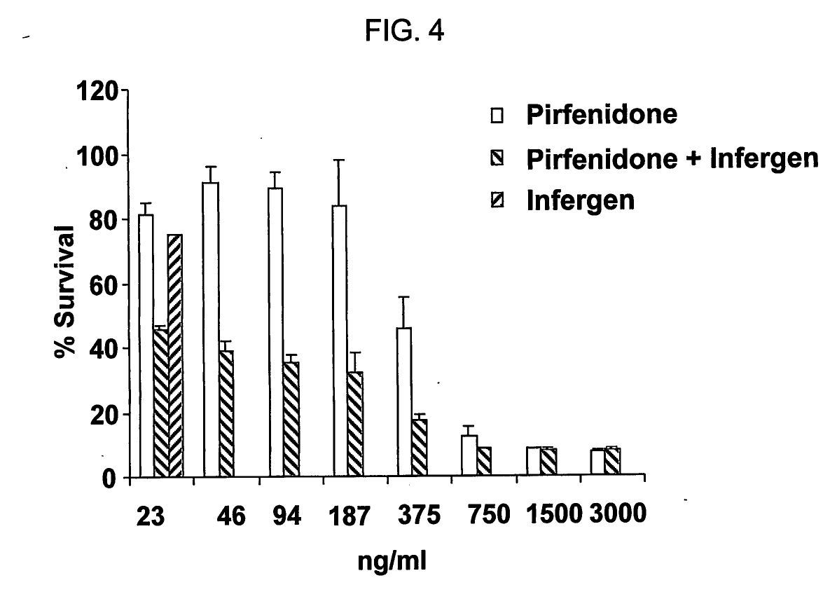

FIG. 4 depicts the antiproliferative effects of various amounts of pirfenidone in combination with 2 ng/ml INFERGEN® on CAOV-3 cells.

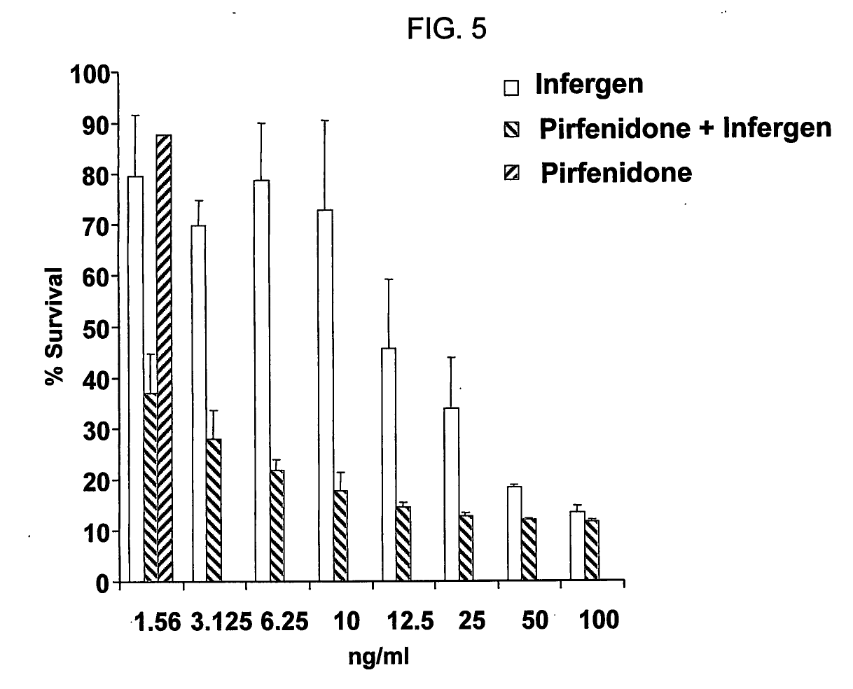

FIG. 5 depicts the antiproliferative effects of various amounts of INFERGEN® in combination with 30 μg/ml pirfenidone on the CAOV-3 cell line.

### Example 3

### Susceptibility of Cancer Cells to Growth Inhibition by INFERGEN® and IP-10

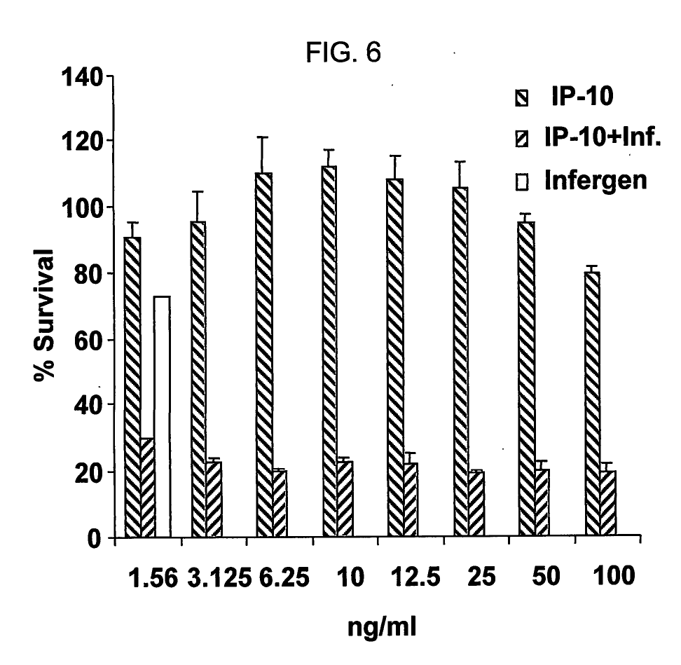

CAOV-3 cells (American Type Culture Collection No. HTB-75) or OVCAR cells were grown in the presence of INFERGEN®, IP-10, or a combination of INFERGEN® and IP-10. Control cell cultures were grown without INFERGEN® or IP-10. The results are shown in FIGS. 6-8.

FIG. 6 depicts the antiproliferative effects of various amounts of IP-10 in combination-with 2 ng/ml INFERGEN® on CAOV-3 cells.

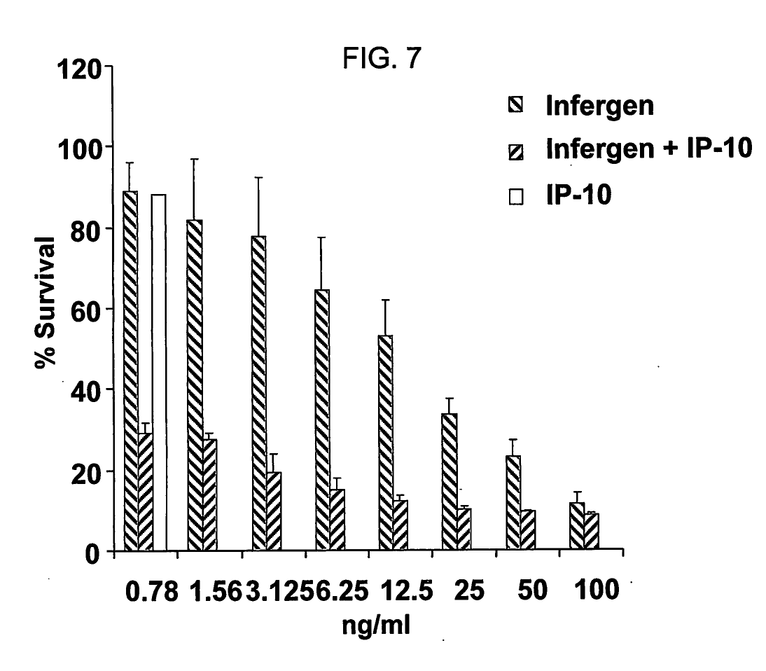

FIG. 7 depicts the antiproliferative effects of various amounts of INFERGEN® in combination with 10 ng/ml IP-10 on the CAOV-3 cell line.

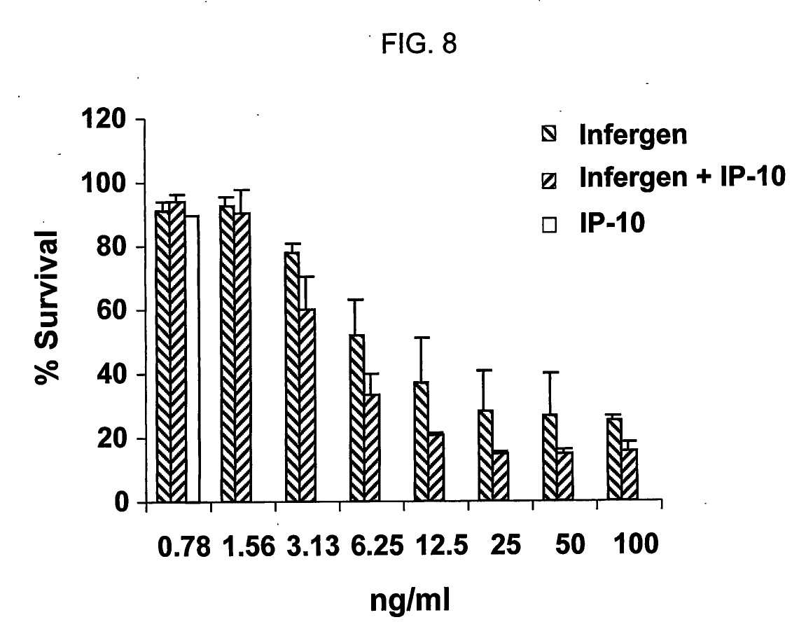

FIG. 8 depicts the antiproliferative effects of various amounts of INFERGEN® in combination with 10 ng/ml IP-10 on the OVCAR cell line.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for treating cancer in an individual, the method comprising administering a therapeutically effective amount of IP-10 and a therapeutically effective amount of pirfenidone or a pirfenidone analog to the individual.

2. The method of claim 1, wherein the treatment is effective in reducing tumor load by at least about 20%.

3. The method of claim 1, wherein the pirfenidone or pirfenidone analog is administered orally in a dosage range of from 100 mg to 1000 mg per day.

4. The method of claim 1, wherein IP-10 is administered in a dosage range of from 0.1 mg/kg body weight to about 10 mg/kg body weight.

5. The method of claim 1, further comprising administering an effective amount of IFN-α.

6. The method of claim 1, further comprising administering an effective amount of an antiproliferative agent selected from an alkylating agent, a nitrosourea, an antimetabolite, an anti-tumor antibody, a steroid hormone, a vinca alkyloid, and a taxane.

7. A method of treating cancer in an individual, the method comprising:
determining the susceptibility of a cancerous cell from the individual to growth inhibition by IP-10 and pirfenidone; and
administering a therapeutically effective amount of IP-10 and a therapeutically effective amount of pirfenidone or a pirfenidone analog to an individual having a tumor susceptible to growth inhibition by IP-10 and pirfenidone.

8.-21. (canceled)

22. A method for treating cancer in an individual, the method comprising administering a therapeutically effective amount of pirfenidone or a pirfenidone analog and a therapeutically effective amount of at least one additional antineoplastic agent or biological response modifier to the individual.

23. The method of claim 22, wherein the cancer is a solid tumor and the treatment is effective in reducing tumor load by at least about 20%.

24. The method of claim 22, wherein the pirfenidone or pirfenidone analog is administered orally in a dosage range of from 100 mg to 1000 mg per day.

25. The method of claim 22, wherein the at least one additional antineoplastic agent or biological response modifier is IFN-γ a.

26. (canceled)

27. The method of claim 22, wherein the at least one additional antineoplastic agent or biological response modifier is selected from an alkylating agent, a nitrosourea, an antimetabolite, an anti-tumor antibody, a steroid hormone, a vinca alkaloid, a platinum complex, and a taxane.

28. (canceled)

29. The method of claim 27, wherein the taxane is paclitaxel or docetaxel.

30. The method of claim 22, wherein the at least one additional antineoplastic agent or biological response modifier is an antiangiogenic agent selected from an anti-VEGF monoclonal antibody or fragment thereof, an anti-bFGF monoclonal antibody or fragment thereof, an anti-bFGF receptor monoclonal antibody or fragment thereof, an anti-TGF-β monoclonal antibody or fragment thereof, and an anti-TGF-β receptor monoclonal antibody or fragment thereof.

31. (canceled)

32. The method of claim 22, wherein the at least one additional antineoplastic agent or biological response modifier is an inhibitor of a receptor tyrosine kinase (RTK).

33. The method of claim 32, wherein the RTK inhibitor is erolotinib or gefitinib.

34.-38. (canceled)

39. The method of claim 22, wherein the at least one additional antineoplastic agent or biological response modifier is an inhibitor of a non-receptor tyrosine kinase.

40. The method of claim 39, wherein the inhibitor of the non-receptor tyrosine kinase is imatinib.

41.-43. (canceled)

44. The method of claim 27, wherein the platinum complex is cisplatin or carboplatin.
